SEQUENCE LISTING (10 sequences)

SEQ ID NO: 1 - DNA, 1938 bp, Artificial Sequence
  Description of Artificial Sequence Synthetic polynucleotide
atggctgatagcaatggaacgattacagtagaagagttaaaaaaacttctagagcaatggaatcttgtaattggctttctatttctaacatggatatgtctattacagtttgcttatgccaatagaaatagatttctttatataataaaacttatctttctatggctattatggcctgttacattagcttgtttcgttctagctgctgtttatagaataaattggataaccggtggaattgcaattgctatggcctgcttagtcggacttatgtggctttcatattttattgcctcatttcgattattcgctagaacacgctcgatgtggagctttaatccagaaactaatatattattaaatgtgccattacatggtacaattttgactagacctcttttagaaagcgaattagttataggtgcagttatcctacgtggacatttaagaattgctggccaccatcttggtagatgtgatatcaaagatttaccaaaagaaataactgtagcaacatctagaacattatcatattataaattgggtgcttcacagagagtggcgggtgattcaggttttgcagcttattctaggtataggattggtaactataaattgaatacggatcacagttcctcaagtgataatattgcacttcttgtacagggtggtagcggtatgtcagataacggtcctcaaaatcaaagaaatgctcctagaataacttttggtggcccaagtgatagtactggtagtaatcagaacggtgagagaagtggagcaagatctaagcaacgcagaccgcaagggctacctaataatactgcgtcatggtttactgctttaacacaacatggtaaagaagatttaaagtttcctcgcggtcagggtgttccaattaatactaatagttcgccagatgatcaaattggttattatcgtcgtgctactagacgaattcgtggtggcgacggaaaaatgaaagatctatctccacgttggtacttttactatttaggtaccggtccagaggctggtttaccttatggtgctaacaaagacgggataatatgggtcgctaccgagggtgcacttaatacgccaaaagatcatatcggaactcgtaacccagcaaataacgctgctattgttttacaattacctcaaggtactacactgcctaaaggtttctatgcagagggctctaggggtggaagccaagcatcaagtcgttcaagttctcgtagcagaaactcttctagaaatagtactcctggctcatcacgtggaacaagtccagcgagaatggctggtaatggcggggatgcagcattagcattgttacttttagatagattaaatcagcttgaatctaaaatgtctggcaaagggcaacaacaacagggtcaaacagtaactaagaaatcagcagcagaggcttcgaagaaacctagacagaagagaactgctacaaaagcgtacaatgttacgcaagcatttggccgcagaggaccagaacagactcaagggaattttggtgatcaagaattaattcgtcaaggtacagattataaacattggccccagatagctcaatttgctccatctgcatctgcattctttggaatgtcaagaattggtatggaagttactcctagtggaacttggctaacttatactggtgctataaagctcgatgataaagatcctaattttaaagatcaagtaattttgttaaacaagcatatagatgcatataaaacatttcctcctactgaaccaaaaaaagataaaaagaaaaaagctgacgaaacacaagctctaccgcaaaggcaaaagaaacaacagacagtaacattattgccagctgctgatttagatgatttttcaaaacaacttcaacaatctatgagtagcgcagatagtactcaagcataa

SEQ ID NO: 2 - DNA, 1998 bp, Artificial Sequence
  Description of Artificial Sequence Synthetic polynucleotide
atgtctgtagcgagtcaatcaataatagcatatacaatgtcattaggcgcagaaaatagtgtggcttattctaataattctatcgcaatccctaccaatttcactataagtgttacaaccgaaatcttaccagttagtatgacaaagacaagtgttgattgtactatgtatatatgtggcgattctactgagtgttctaatctcttattacaatatggttcgttttgtactcagttaaatcgagctcttacaggtatagctgtcgagcaagataagaatacccaggaagtctttgcacaggttaaacaaatttataaaactccaccaatcaaagattttggtgggtttaacttttctcaaatactacctgatccatctaaaccctctaaacgtagttttattgaagatttactttttaataaagtaactctagctgatgctggtttcattaaacaatacggcgattgtttgggtgatatagcggcacgtgatttaatatgcgcacagaaattcaacggtctgacagtcctacctccattattgacagatgaaatgattgctcaatatacatcagcattgcttgctggcactatcacgagtggatggacttttggtgctggcgctgctttacaaattccatttgccatgcaaatggcttatagatttaatggtattggtgttacacaaaatgttttatatgagaatcaaaagttaatagctaaccaatttaactctgcaattggcaagattcaggattcattatctagtacagcgagtgctttaggtaaactacaagatgtagtgaatcagaatgctcaagcactcaatactttggttaaacaattaagttcaaattttggtgcaatttcaagtgtactaaatgatattctaagtcgcttagataaagttgaggctgaagtacaaatcgatagactaattacaggtagattacagtcattacaaacttatgttactcaacagttaattagagctgcagaaataagagcatctgcaaatttggcagccactaagatgagtgagtgtgtccttggacaatcaaaacgtgtagatttttgcggaaagggatatcacttaatgtcatttccgcaatctgcacctcatggtgtcgtgtttcttcatgttacttacgttccggctcaagagaaaaacttcactacggctccagcgatttgtcatgatggtaaagctcattttcctcgtgagggtgtatttgtatcaaatggaacacattggtttgttactcaaagaaatttttatgagccacaaataataactacagataatacttttgttagcggtaactgtgacgtagttataggaatcgtaaacaacacagtgtatgatccattacaaccagagttagattcttttaaagaagaacttgataagtatttcaaaaatcatactagccctgatgttgaccttggtgacatatcaggcataaatgcatcagttgttaatattcaaaaagaaatagataggcttaatgaagttgctaaaaatcttaatgaatctttaatagatctacaagaacttggaaaatacgaacaatatataaaatggccttggtatatatggttagggtttattgctggtcttattgctattgtaatggtaactattatgctatgttgtatgacatcatgctgtagctgtctaaagggttgttgtagttgtggttcatgttgcaaatttgatgaagatgatagtgagccagttcttaaaggtgtaaaattggggggatctggaatgtacagctttgtgtcagaagaaaccggtacactaattgttaatagcgttttactttttctggcttttgttgtgtttcttctagtaacattggccatcttgactgcactaagactttgtgcttattgctgtaatattgttaatgtttcattagtaaaacctagcttttatgtttattcgagagtcaaaaacctaaattccagtagagtacctgatttattagtataa

SEQ ID NO: 3 - DNA, 3822 bp, Artificial Sequence
  Description of Artificial Sequence Synthetic polynucleotide
atgtttgtgtttttagttcttttaccgttagtttcaagtcaatgtgtgaacttaactacacgcacacaacttcctccagcatatacaaatagttttactagaggtgtatattatcctgataaagtattccgtagttctgttctacattctacacaagatttgtttttaccgtttttcagtaatgtcacttggttccatgctattcatgtttctgggacaaacggtacaaaaagatttgataaccctgttttaccatttaatgatggtgtatattttgcttcaactgagaaaagcaatataattagaggttggattttcggaactaccctggatagcaagacgcaaagtttattgatcgtaaacaatgctacaaacgtcgtaattaaagtatgtgaatttcaattttgtaatgacccttttttaggagtctattatcataaaaataataaatcttggatggagtctgaatttagagtttattctagcgctaataactgtacatttgaatatgtttcacaaccttttttaatggatctagaaggtaaacagggtaattttaaaaatcttcgtgagtttgtttttaagaacatagatggatatttcaaaatatattcaaaacatactcctattaatctagttagagatcttccacaaggcttttctgctctagaaccattagttgatttaccaataggtataaatataactcgtttccaaactttactagcccttcaccgttcgtacttaacgcctggggattcttctagtggttggactgctggcgctgcagcatattatgttggatatctacaacctagaacatttttattgaaatacaacgaaaacggaactataactgacgctgttgattgtgcacttgatccattaagtgagactaaatgtactctaaaaagttttactgttgaaaagggaatttatcaaacatcaaattttcgcgttcaaccaacggaaagtattgtacgttttccgaacataaccaatttatgtcctttcggtgaggtatttaacgcaactcgttttgcgagcgtatatgcttggaatagaaaaagaattagcaattgtgttgctgattattcggtcttatacaatagtgcttcgtttagcacttttaaatgttacggagtaagtccaacaaagttaaatgatctatgtttcactaatgtgtatgctgattcttttgttattagaggtgatgaagttcgacaaattgctccaggtcaaactggcaaaattgcggactataattataagctacctgatgattttactggctgtgtgattgcatggaatagtaataatctagattcgaaagtcggtgggaattataattatctttatagactatttagaaaatctaatttgaaaccatttgagagagatatatcaacagaaatttaccaggctggcagcacaccttgcaacggcgtagaaggttttaattgttattttccactacaaagttatggttttcaaccaactaatggcgtcgggtatcaaccatatagagttgtcgtactttcctttgaattacttcatgcaccagctaccgtttgtgggccaaagaaatcaactaatcttgtaaagaataaatgcgtcaattttaattttaatggccttacaggcactggagttttaacagaatccaataaaaaatttttaccttttcagcaatttggtagagatatagctgatactactgatgctgtaagagatcctcaaactctagagattttagatattaccccgtgttcatttggaggcgtaagcgttataactccaggcacgaacacatcaaatcaagttgctgtactatatcaagatgttaattgcacagaagtgcctgttgccattcatgcagatcaacttactcctacatggcgtgtatattctaccggatcaaatgtatttcagactagagctggttgtttaataggcgcagaacatgtaaataatagttatgagtgtgatataccaattggtgcaggaatatgtgcatcatatcagacacagacaaatagtcctcgtcgcgcaagatcagtagcatcacaatcgattatagcttatacaatgtctttaggtgcggaaaatagtgtggcttattctaataattctatcgcaatccctaccaatttcactataagtgttacaaccgaaatcttaccagttagtatgacaaagacaagtgttgattgtactatgtatatatgtggcgattctactgagtgttctaatctcttattacaatatggttcgttttgtactcagttaaatcgagctcttacaggtatagctgtcgagcaagataagaatacccaggaagtctttgcacaggttaaacaaatttataaaactccaccaatcaaagattttggtgggtttaacttttctcaaatactacctgatccatctaaaccctctaaacgtagttttattgaagatttactttttaataaagtaactctagctgatgctggtttcattaaacaatacggcgattgtttgggtgatatagcggcacgtgatttaatatgcgcacagaaattcaacggtctgacagtcctacctccattattgacagatgaaatgattgctcaatatacatcagcattgcttgctggcactatcacgagtggatggacttttggtgctggcgctgctttacaaattccatttgccatgcaaatggcttatagatttaatggtattggtgttacacaaaatgttttatatgagaatcaaaagttaatagctaaccaatttaactctgcaattggcaagattcaggattcattatctagtacagcgagtgctttaggtaaactacaagatgtagtgaatcagaatgctcaagcactcaatactttggttaaacaattaagttcaaattttggtgcaatttcaagtgtactaaatgatattctaagtcgcttagatcctccagaggctgaagtacaaatcgatagactaattacaggtagattacagtcattacaaacttatgttactcaacagttaattagagctgcagaaataagagcatctgcaaatttggcagccactaagatgagtgagtgtgtccttggacaatcaaaacgtgtagatttttgcggaaagggatatcacttaatgtcatttccgcaatctgcacctcatggtgtcgtgtttcttcatgttacttacgttccggctcaagagaaaaacttcactacggctccagcgatttgtcatgatggtaaagctcattttcctcgtgagggtgtatttgtatcaaatggaacacattggtttgttactcaaagaaatttttatgagccacaaataataactacagataatacttttgttagcggtaactgtgacgtagttataggaatcgtaaacaacacagtgtatgatccattacaaccagagttagattcttttaaagaagaacttgataagtatttcaaaaatcatactagccctgatgttgaccttggtgacatatcaggcataaatgcatcagttgttaatattcaaaaagaaatagataggcttaatgaagttgctaaaaatcttaatgaatctttaatagatctacaagaacttggaaaatacgaacaatatataaaatggccttggtatatatggttagggtttattgctggtcttattgctattgtaatggtaactattatgctatgttgtatgacatcatgctgtagctgtctaaagggttgttgtagttgtggttcatgttgcaaatttgatgaagatgatagtgagccagttcttaaaggtgtaaaattgcattacacatga

SEQ ID NO: 4 - DNA, 8340 bp, Artificial Sequence
  Description of Artificial Sequence Synthetic polynucleotide
ggtacctggttactattgccatcatcacaatattaaaattaattttcttcatttatttttcttaaatattattattaaaaatagtaaatttaacttatctaaaaatagcataatatcatttttattaaaatatctaggttgaattcttagatattttgatatataattagatactaaattgataacttataaagaattaaattttcttttgtatgctaacttgattgctaatatgaattatactagttagtatgttgattataatgattagagttttaaataatggaggtaacaataggaggtacgtaatggattataaagatcacgatggtgattacaaagaccatgatatagattataaggatgacgatgataagcatcatcatcaccaccatcatcatggaggtggttcaatggctgatagcaatggaacgattacagtagaagagttaaaaaaacttctagagcaatggaatcttgtaattggctttctatttctaacatggatatgtctattacagtttgcttatgccaatagaaatagatttctttatataataaaacttatctttctatggctattatggcctgttacattagcttgtttcgttctagctgctgtttatagaataaattggataaccggtggaattgcaattgctatggcctgcttagtcggacttatgtggctttcatattttattgcctcatttcgattattcgctagaacacgctcgatgtggagctttaatccagaaactaatatattattaaatgtgccattacatggtacaattttgactagacctcttttagaaagcgaattagttataggtgcagttatcctacgtggacatttaagaattgctggccaccatcttggtagatgtgatatcaaagatttaccaaaagaaataactgtagcaacatctagaacattatcatattataaattgggtgcttcacagagagtggcgggtgattcaggttttgcagcttattctaggtataggattggtaactataaattgaatacggatcacagttcctcaagtgataatattgcacttcttgtacagggtggtagcggtatgtcagataacggtcctcaaaatcaaagaaatgctcctagaataacttttggtggcccaagtgatagtactggtagtaatcagaacggtgagagaagtggagcaagatctaagcaacgcagaccgcaagggctacctaataatactgcgtcatggtttactgctttaacacaacatggtaaagaagatttaaagtttcctcgcggtcagggtgttccaattaatactaatagttcgccagatgatcaaattggttattatcgtcgtgctactagacgaattcgtggtggcgacggaaaaatgaaagatctatctccacgttggtacttttactatttaggtaccggtccagaggctggtttaccttatggtgctaacaaagacgggataatatgggtcgctaccgagggtgcacttaatacgccaaaagatcatatcggaactcgtaacccagcaaataacgctgctattgttttacaattacctcaaggtactacactgcctaaaggtttctatgcagagggctctaggggtggaagccaagcatcaagtcgttcaagttctcgtagcagaaactcttctagaaatagtactcctggctcatcacgtggaacaagtccagcgagaatggctggtaatggcggggatgcagcattagcattgttacttttagatagattaaatcagcttgaatctaaaatgtctggcaaagggcaacaacaacagggtcaaacagtaactaagaaatcagcagcagaggcttcgaagaaacctagacagaagagaactgctacaaaagcgtacaatgttacgcaagcatttggccgcagaggaccagaacagactcaagggaattttggtgatcaagaattaattcgtcaaggtacagattataaacattggccccagatagctcaatttgctccatctgcatctgcattctttggaatgtcaagaattggtatggaagttactcctagtggaacttggctaacttatactggtgctataaagctcgatgataaagatcctaattttaaagatcaagtaattttgttaaacaagcatatagatgcatataaaacatttcctcctactgaaccaaaaaaagataaaaagaaaaaagctgacgaaacacaagctctaccgcaaaggcaaaagaaacaacagacagtaacattattgccagctgctgatttagatgatttttcaaaacaacttcaacaatctatgagtagcgcagatagtactcaagcataaggttaaggatccactagctcgtttcaaattaccgatgatatcggaccgttccaacttaccgaccagttcggcaggtatgtatttgcgtgcattcctatccaaaaaaacatcaagccaaaagcttgaaaaaacttacaacacagctcaacagagctagattgtaaaaccctgctttgttaagcagaacgcaaaaattgaatgacttatagtcatatcgcttcgaccctcgtagattagtagccttgagctattaactggttgaaacacttaccaaataaagattaaaagcgataaaaatgaaagataaagcagctaaaaacagagattttagaaagactattttatcagtgttacaacgcaataaagatggctcttttgctacgcaagcaaatagaaagtctattctgttgcaggcaactaaagaccttaaaaaggtagggtttagcaaggttacagccgaaaacttcggtaataagcattgctatgcacttagagaccattggagagcccaaggattagctacagcaacgataaaaaatcgtttagcttgtctaaggtggttaggcgagaaaatgggcaaagaactacccgataatcgaaaattagagattgagaacaggaagtatagcgataattcaatcaataaagcccaagaaatcgattttaaggcgatttctgccttaactgataggcaagccctagcaatacaattacagcgcgaatttgggcttcgtagagaagaaagtttgaagtttcagcctagttatgcaatcaaagagcataaaatcgagcttaaaagctcttggacaaagggtggaagaccacgagaaatcccaattttgaatgaaaaacagagagaattgttagaaaaagtaaaagaggtagcaggtaaaggctctctaattgagagcgaaaagtcttataagcaagcaatggaacatttcacgactcgctgtcaaagagcagggattaagaatgttcatggctttagacatgcgtatgctcaagatagatataggcaattaacagggcgtgagtgtcctaaaaatggtggattaacatctaagcagctaacacctgagcaaaagcaacaagactatgaagctagaatgactattagtgaagagttaggtcatggtagagaagatgtaacagtcaactacttaggcagataaaaagcaatatagctatagaagaaaagaaagctattttacatagtagatcgactcttcttagggattttatattttttgataaatcatctattttgctagttaaatcatcaaatttatcatcttgttgtttgactaaatctaagaatctattctcttttttaaaatcgttcatgcaaaccgcctatagctttcttctttttctgaaattatttgtcttcacaccataattaaattcccatttttataagtaaagtcttttaaaagcttgtcagtctcttctctagaaatgtaccaaattttacctattttaggatacttttcatgaagttcttctatttttccccagtcctttaatagtctacctttagagtctcgtaaatagttatctttgtgacaggggcctcttttatcttttttaatgtaactatatgttattccaacgtcactattactattatccaaatcttttttagcatgccagtaagaactttcataacttaactctatctttcgacctctttgatatacaacaataaagctatagccagtagtaacaacctgttttacttttgttaaatctattaacttcttatttatttttttatgtttttttgaaaatttaaatatttctatattcattcctacacttcctcaaatccaaatggtagcttatgattctcttctggtttcttttctaatttttttatatttgcaataaaaactctttttctatctttgatttttttattgtcccaattcctccaagtatcatcacaaaccctttcaatatcatgtaaatgatgatgtctaaatattgatctgacataatacagatctaggtctagttcatcacttaacacaacttctctaagtctttcagatgcttcgattggtatgtaatcctctttatttttagtatctaaaagcttttgcttaaattcttcttctgtctctgctaccttactaactgtaaacttgatatttgtaatcttacgaccatgttttctgtgatgatccttgtcatcataggttacaaaaatatccgataattgattaatctcttctagtgctggtaataggaacttatttttaaaatttgaatatctgttgctgtaacttttaggtaaatcaaaatcattaatcatatcatcgacatacaatacgcaatcaactatattagcataccctgcttgttcgcctaatttgcttttgagaagtaagtataatctgcttgaatacttacttttaaatgaaaatagtaactgtctttctgctttagtaaagtactcttgtagttgtatcatgtgtggcattaatgaccaatgaaactcgcaaattaaagcactgcttttagggtctgcttcaatatatgcaaaccagttagctatcttcgtttgttctttattcagccatactggcttagacattattgagtgcattaattgcttcaatctcactctgttatgcttaacccctgtagctttttcaagatcagataggcttatcttatacctgtgaaactctttatcttctcttttaaccattgaggcaactaagaatattaagttttgttcttcttttgtaaggctatactttcctgcaacaagagtattagacatagctatttctttgccagcatttacatttttaacttctttcatagaactagagtcattatctcgatatacaaattctataaaacttctattagtaaaacaactacttcataaaaaaaagtagttttaacgatacaaaaagtagttttaaattcaaaaagtgatacaaaaagtagttttaaattcaaaaagtgatacaaaaagtagttttaaattttttaaaaaagtgcttcaaagccttatgtagcaatacttacagaggattaaaaaaaaatctgacaatatataaagagaatatataaagagaatatcttaggggattttaaaaaaatcccacagactcaaagacttttttgactttttaaatcctagaaactatactttaagtacttatttaagtacatggatttagattatgcaaaccgttaattattcaacttttagaaatgaactatctgattcaatggatagagtaacaaaaaatcatagtcctatgattgtaactagaggttcaaaaaaagaagcagttgttatgatgtcgttagaggattcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaattcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagaacatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagaacttggcggcgaatgggctgaccgcttcctcgtgctttacggtatcgccgctcccgattcgcagcgcatcgccttctatcgccttcttgacgagttcttctgaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaaagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctt

SEQ ID NO: 5 - DNA, 10224 bp, Artificial Sequence
  Description of Artificial Sequence Synthetic polynucleotide
ggtacctggttactattgccatcatcacaatattaaaattaattttcttcatttatttttcttaaatattattattaaaaatagtaaatttaacttatctaaaaatagcataatatcatttttattaaaatatctaggttgaattcttagatattttgatatataattagatactaaattgataacttataaagaattaaattttcttttgtatgctaacttgattgctaatatgaattatactagttagtatgttgattataatgattagagttttaaataatggaggtaacaataggaggtacgtaatggattataaagatcacgatggtgattacaaagaccatgatatagattataaggatgacgatgataagcatcatcatcaccaccatcatcatggaggtggttcaatgtttgtgtttttagttcttttaccgttagtttcaagtcaatgtgtgaacttaactacacgcacacaacttcctccagcatatacaaatagttttactagaggtgtatattatcctgataaagtattccgtagttctgttctacattctacacaagatttgtttttaccgtttttcagtaatgtcacttggttccatgctattcatgtttctgggacaaacggtacaaaaagatttgataaccctgttttaccatttaatgatggtgtatattttgcttcaactgagaaaagcaatataattagaggttggattttcggaactaccctggatagcaagacgcaaagtttattgatcgtaaacaatgctacaaacgtcgtaattaaagtatgtgaatttcaattttgtaatgacccttttttaggagtctattatcataaaaataataaatcttggatggagtctgaatttagagtttattctagcgctaataactgtacatttgaatatgtttcacaaccttttttaatggatctagaaggtaaacagggtaattttaaaaatcttcgtgagtttgtttttaagaacatagatggatatttcaaaatatattcaaaacatactcctattaatctagttagagatcttccacaaggcttttctgctctagaaccattagttgatttaccaataggtataaatataactcgtttccaaactttactagcccttcaccgttcgtacttaacgcctggggattcttctagtggttggactgctggcgctgcagcatattatgttggatatctacaacctagaacatttttattgaaatacaacgaaaacggaactataactgacgctgttgattgtgcacttgatccattaagtgagactaaatgtactctaaaaagttttactgttgaaaagggaatttatcaaacatcaaattttcgcgttcaaccaacggaaagtattgtacgttttccgaacataaccaatttatgtcctttcggtgaggtatttaacgcaactcgttttgcgagcgtatatgcttggaatagaaaaagaattagcaattgtgttgctgattattcggtcttatacaatagtgcttcgtttagcacttttaaatgttacggagtaagtccaacaaagttaaatgatctatgtttcactaatgtgtatgctgattcttttgttattagaggtgatgaagttcgacaaattgctccaggtcaaactggcaaaattgcggactataattataagctacctgatgattttactggctgtgtgattgcatggaatagtaataatctagattcgaaagtcggtgggaattataattatctttatagactatttagaaaatctaatttgaaaccatttgagagagatatatcaacagaaatttaccaggctggcagcacaccttgcaacggcgtagaaggttttaattgttattttccactacaaagttatggttttcaaccaactaatggcgtcgggtatcaaccatatagagttgtcgtactttcctttgaattacttcatgcaccagctaccgtttgtgggccaaagaaatcaactaatcttgtaaagaataaatgcgtcaattttaattttaatggccttacaggcactggagttttaacagaatccaataaaaaatttttaccttttcagcaatttggtagagatatagctgatactactgatgctgtaagagatcctcaaactctagagattttagatattaccccgtgttcatttggaggcgtaagcgttataactccaggcacgaacacatcaaatcaagttgctgtactatatcaagatgttaattgcacagaagtgcctgttgccattcatgcagatcaacttactcctacatggcgtgtatattctaccggatcaaatgtatttcagactagagctggttgtttaataggcgcagaacatgtaaataatagttatgagtgtgatataccaattggtgcaggaatatgtgcatcatatcagacacagacaaatagtcctcgtcgcgcaagatcagtagcatcacaatcgattatagcttatacaatgtctttaggtgcggaaaatagtgtggcttattctaataattctatcgcaatccctaccaatttcactataagtgttacaaccgaaatcttaccagttagtatgacaaagacaagtgttgattgtactatgtatatatgtggcgattctactgagtgttctaatctcttattacaatatggttcgttttgtactcagttaaatcgagctcttacaggtatagctgtcgagcaagataagaatacccaggaagtctttgcacaggttaaacaaatttataaaactccaccaatcaaagattttggtgggtttaacttttctcaaatactacctgatccatctaaaccctctaaacgtagttttattgaagatttactttttaataaagtaactctagctgatgctggtttcattaaacaatacggcgattgtttgggtgatatagcggcacgtgatttaatatgcgcacagaaattcaacggtctgacagtcctacctccattattgacagatgaaatgattgctcaatatacatcagcattgcttgctggcactatcacgagtggatggacttttggtgctggcgctgctttacaaattccatttgccatgcaaatggcttatagatttaatggtattggtgttacacaaaatgttttatatgagaatcaaaagttaatagctaaccaatttaactctgcaattggcaagattcaggattcattatctagtacagcgagtgctttaggtaaactacaagatgtagtgaatcagaatgctcaagcactcaatactttggttaaacaattaagttcaaattttggtgcaatttcaagtgtactaaatgatattctaagtcgcttagatcctccagaggctgaagtacaaatcgatagactaattacaggtagattacagtcattacaaacttatgttactcaacagttaattagagctgcagaaataagagcatctgcaaatttggcagccactaagatgagtgagtgtgtccttggacaatcaaaacgtgtagatttttgcggaaagggatatcacttaatgtcatttccgcaatctgcacctcatggtgtcgtgtttcttcatgttacttacgttccggctcaagagaaaaacttcactacggctccagcgatttgtcatgatggtaaagctcattttcctcgtgagggtgtatttgtatcaaatggaacacattggtttgttactcaaagaaatttttatgagccacaaataataactacagataatacttttgttagcggtaactgtgacgtagttataggaatcgtaaacaacacagtgtatgatccattacaaccagagttagattcttttaaagaagaacttgataagtatttcaaaaatcatactagccctgatgttgaccttggtgacatatcaggcataaatgcatcagttgttaatattcaaaaagaaatagataggcttaatgaagttgctaaaaatcttaatgaatctttaatagatctacaagaacttggaaaatacgaacaatatataaaatggccttggtatatatggttagggtttattgctggtcttattgctattgtaatggtaactattatgctatgttgtatgacatcatgctgtagctgtctaaagggttgttgtagttgtggttcatgttgcaaatttgatgaagatgatagtgagccagttcttaaaggtgtaaaattgcattacacatgaggttaaggatccactagctcgtttcaaattaccgatgatatcggaccgttccaacttaccgaccagttcggcaggtatgtatttgcgtgcattcctatccaaaaaaacatcaagccaaaagcttgaaaaaacttacaacacagctcaacagagctagattgtaaaaccctgctttgttaagcagaacgcaaaaattgaatgacttatagtcatatcgcttcgaccctcgtagattagtagccttgagctattaactggttgaaacacttaccaaataaagattaaaagcgataaaaatgaaagataaagcagctaaaaacagagattttagaaagactattttatcagtgttacaacgcaataaagatggctcttttgctacgcaagcaaatagaaagtctattctgttgcaggcaactaaagaccttaaaaaggtagggtttagcaaggttacagccgaaaacttcggtaataagcattgctatgcacttagagaccattggagagcccaaggattagctacagcaacgataaaaaatcgtttagcttgtctaaggtggttaggcgagaaaatgggcaaagaactacccgataatcgaaaattagagattgagaacaggaagtatagcgataattcaatcaataaagcccaagaaatcgattttaaggcgatttctgccttaactgataggcaagccctagcaatacaattacagcgcgaatttgggcttcgtagagaagaaagtttgaagtttcagcctagttatgcaatcaaagagcataaaatcgagcttaaaagctcttggacaaagggtggaagaccacgagaaatcccaattttgaatgaaaaacagagagaattgttagaaaaagtaaaagaggtagcaggtaaaggctctctaattgagagcgaaaagtcttataagcaagcaatggaacatttcacgactcgctgtcaaagagcagggattaagaatgttcatggctttagacatgcgtatgctcaagatagatataggcaattaacagggcgtgagtgtcctaaaaatggtggattaacatctaagcagctaacacctgagcaaaagcaacaagactatgaagctagaatgactattagtgaagagttaggtcatggtagagaagatgtaacagtcaactacttaggcagataaaaagcaatatagctatagaagaaaagaaagctattttacatagtagatcgactcttcttagggattttatattttttgataaatcatctattttgctagttaaatcatcaaatttatcatcttgttgtttgactaaatctaagaatctattctcttttttaaaatcgttcatgcaaaccgcctatagctttcttctttttctgaaattatttgtcttcacaccataattaaattcccatttttataagtaaagtcttttaaaagcttgtcagtctcttctctagaaatgtaccaaattttacctattttaggatacttttcatgaagttcttctatttttccccagtcctttaatagtctacctttagagtctcgtaaatagttatctttgtgacaggggcctcttttatcttttttaatgtaactatatgttattccaacgtcactattactattatccaaatcttttttagcatgccagtaagaactttcataacttaactctatctttcgacctctttgatatacaacaataaagctatagccagtagtaacaacctgttttacttttgttaaatctattaacttcttatttatttttttatgtttttttgaaaatttaaatatttctatattcattcctacacttcctcaaatccaaatggtagcttatgattctcttctggtttcttttctaatttttttatatttgcaataaaaactctttttctatctttgatttttttattgtcccaattcctccaagtatcatcacaaaccctttcaatatcatgtaaatgatgatgtctaaatattgatctgacataatacagatctaggtctagttcatcacttaacacaacttctctaagtctttcagatgcttcgattggtatgtaatcctctttatttttagtatctaaaagcttttgcttaaattcttcttctgtctctgctaccttactaactgtaaacttgatatttgtaatcttacgaccatgttttctgtgatgatccttgtcatcataggttacaaaaatatccgataattgattaatctcttctagtgctggtaataggaacttatttttaaaatttgaatatctgttgctgtaacttttaggtaaatcaaaatcattaatcatatcatcgacatacaatacgcaatcaactatattagcataccctgcttgttcgcctaatttgcttttgagaagtaagtataatctgcttgaatacttacttttaaatgaaaatagtaactgtctttctgctttagtaaagtactcttgtagttgtatcatgtgtggcattaatgaccaatgaaactcgcaaattaaagcactgcttttagggtctgcttcaatatatgcaaaccagttagctatcttcgtttgttctttattcagccatactggcttagacattattgagtgcattaattgcttcaatctcactctgttatgcttaacccctgtagctttttcaagatcagataggcttatcttatacctgtgaaactctttatcttctcttttaaccattgaggcaactaagaatattaagttttgttcttcttttgtaaggctatactttcctgcaacaagagtattagacatagctatttctttgccagcatttacatttttaacttctttcatagaactagagtcattatctcgatatacaaattctataaaacttctattagtaaaacaactacttcataaaaaaaagtagttttaacgatacaaaaagtagttttaaattcaaaaagtgatacaaaaagtagttttaaattcaaaaagtgatacaaaaagtagttttaaattttttaaaaaagtgcttcaaagccttatgtagcaatacttacagaggattaaaaaaaaatctgacaatatataaagagaatatataaagagaatatcttaggggattttaaaaaaatcccacagactcaaagacttttttgactttttaaatcctagaaactatactttaagtacttatttaagtacatggatttagattatgcaaaccgttaattattcaacttttagaaatgaactatctgattcaatggatagagtaacaaaaaatcatagtcctatgattgtaactagaggttcaaaaaaagaagcagttgttatgatgtcgttagaggattcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaattcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagaacatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagaacttggcggcgaatgggctgaccgcttcctcgtgctttacggtatcgccgctcccgattcgcagcgcatcgccttctatcgccttcttgacgagttcttctgaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaaagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctt

SEQ ID NO: 6 - DNA, 8460 bp, Artificial Sequence
  Description of Artificial Sequence Synthetic polynucleotide
tgaggttaaggatccactagctcgtttcaaattaccgatgatatcggaccgttccaacttaccgaccagttcggcaggtatgtatttgcgtgcattcctatccaaaaaaacatcaagccaaaagcttgaaaaaacttacaacacagctcaacagagctagattgtaaaaccctgctttgttaagcagaacgcaaaaattgaatgacttatagtcatatcgcttcgaccctcgtagattagtagccttgagctattaactggttgaaacacttaccaaataaagattaaaagcgataaaaatgaaagataaagcagctaaaaacagagattttagaaagactattttatcagtgttacaacgcaataaagatggctcttttgctacgcaagcaaatagaaagtctattctgttgcaggcaactaaagaccttaaaaaggtagggtttagcaaggttacagccgaaaacttcggtaataagcattgctatgcacttagagaccattggagagcccaaggattagctacagcaacgataaaaaatcgtttagcttgtctaaggtggttaggcgagaaaatgggcaaagaactacccgataatcgaaaattagagattgagaacaggaagtatagcgataattcaatcaataaagcccaagaaatcgattttaaggcgatttctgccttaactgataggcaagccctagcaatacaattacagcgcgaatttgggcttcgtagagaagaaagtttgaagtttcagcctagttatgcaatcaaagagcataaaatcgagcttaaaagctcttggacaaagggtggaagaccacgagaaatcccaattttgaatgaaaaacagagagaattgttagaaaaagtaaaagaggtagcaggtaaaggctctctaattgagagcgaaaagtcttataagcaagcaatggaacatttcacgactcgctgtcaaagagcagggattaagaatgttcatggctttagacatgcgtatgctcaagatagatataggcaattaacagggcgtgagtgtcctaaaaatggtggattaacatctaagcagctaacacctgagcaaaagcaacaagactatgaagctagaatgactattagtgaagagttaggtcatggtagagaagatgtaacagtcaactacttaggcagataaaaagcaatatagctatagaagaaaagaaagctattttacatagtagatcgactcttcttagggattttatattttttgataaatcatctattttgctagttaaatcatcaaatttatcatcttgttgtttgactaaatctaagaatctattctcttttttaaaatcgttcatgcaaaccgcctatagctttcttctttttctgaaattatttgtcttcacaccataattaaattcccatttttataagtaaagtcttttaaaagcttgtcagtctcttctctagaaatgtaccaaattttacctattttaggatacttttcatgaagttcttctatttttccccagtcctttaatagtctacctttagagtctcgtaaatagttatctttgtgacaggggcctcttttatcttttttaatgtaactatatgttattccaacgtcactattactattatccaaatcttttttagcatgccagtaagaactttcataacttaactctatctttcgacctctttgatatacaacaataaagctatagccagtagtaacaacctgttttacttttgttaaatctattaacttcttatttatttttttatgtttttttgaaaatttaaatatttctatattcattcctacacttcctcaaatccaaatggtagcttatgattctcttctggtttcttttctaatttttttatatttgcaataaaaactctttttctatctttgatttttttattgtcccaattcctccaagtatcatcacaaaccctttcaatatcatgtaaatgatgatgtctaaatattgatctgacataatacagatctaggtctagttcatcacttaacacaacttctctaagtctttcagatgcttcgattggtatgtaatcctctttatttttagtatctaaaagcttttgcttaaattcttcttctgtctctgctaccttactaactgtaaacttgatatttgtaatcttacgaccatgttttctgtgatgatccttgtcatcataggttacaaaaatatccgataattgattaatctcttctagtgctggtaataggaacttatttttaaaatttgaatatctgttgctgtaacttttaggtaaatcaaaatcattaatcatatcatcgacatacaatacgcaatcaactatattagcataccctgcttgttcgcctaatttgcttttgagaagtaagtataatctgcttgaatacttacttttaaatgaaaatagtaactgtctttctgctttagtaaagtactcttgtagttgtatcatgtgtggcattaatgaccaatgaaactcgcaaattaaagcactgcttttagggtctgcttcaatatatgcaaaccagttagctatcttcgtttgttctttattcagccatactggcttagacattattgagtgcattaattgcttcaatctcactctgttatgcttaacccctgtagctttttcaagatcagataggcttatcttatacctgtgaaactctttatcttctcttttaaccattgaggcaactaagaatattaagttttgttcttcttttgtaaggctatactttcctgcaacaagagtattagacatagctatttctttgccagcatttacatttttaacttctttcatagaactagagtcattatctcgatatacaaattctataaaacttctattagtaaaacaactacttcataaaaaaaagtagttttaacgatacaaaaagtagttttaaattcaaaaagtgatacaaaaagtagttttaaattcaaaaagtgatacaaaaagtagttttaaattttttaaaaaagtgcttcaaagccttatgtagcaatacttacagaggattaaaaaaaaatctgacaatatataaagagaatatataaagagaatatcttaggggattttaaaaaaatcccacagactcaaagacttttttgactttttaaatcctagaaactatactttaagtacttatttaagtacatggatttagattatgcaaaccgttaattattcaacttttagaaatgaactatctgattcaatggatagagtaacaaaaaatcatagtcctatgattgtaactagaggttcaaaaaaagaagcagttgttatgatgtcgttagaggattcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaattcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagaacatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagaacttggcggcgaatgggctgaccgcttcctcgtgctttacggtatcgccgctcccgattcgcagcgcatcgccttctatcgccttcttgacgagttcttctgaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaaagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttggtacctggttactattgccatcatcacaatattaaaattaattttcttcatttatttttcttaaatattattattaaaaatagtaaatttaacttatctaaaaatagcataatatcatttttattaaaatatctaggttgaattcttagatattttgatatataattagatactaaattgataacttataaagaattaaattttcttttgtatgctaacttgattgctaatatgaattatactagttagtatgttgattataatgattagagttttaaataatggaggtaacaataggaggtacgtaatggattataaagatcacgatggtgattacaaagaccatgatatagattataaggatgacgatgataagcatcatcatcaccaccatcatcatggaggtggttcaatgtttgtgtttttagttcttttaccgttagtttcaagtcaatgtgtgaacttaactacacgcacacaacttcctccagcatatacaaatagttttactagaggtgtatattatcctgataaagtattccgtagttctgttctacattctacacaagatttgtttttaccgtttttcagtaatgtcacttggttccatgctattcatgtttctgggacaaacggtacaaaaagatttgataaccctgttttaccatttaatgatggtgtatattttgcttcaactgagaaaagcaatataattagaggttggattttcggaactaccctggatagcaagacgcaaagtttattgatcgtaaacaatgctacaaacgtcgtaattaaagtatgtgaatttcaattttgtaatgacccttttttaggagtctattatcataaaaataataaatcttggatggagtctgaatttagagtttattctagcgctaataactgtacatttgaatatgtttcacaaccttttttaatggatctagaaggtaaacagggtaattttaaaaatcttcgtgagtttgtttttaagaacatagatggatatttcaaaatatattcaaaacatactcctattaatctagttagagatcttccacaaggcttttctgctctagaaccattagttgatttaccaataggtataaatataactcgtttccaaactttactagcccttcaccgttcgtacttaacgcctggggattcttctagtggttggactgctggcgctgcagcatattatgttggatatctacaacctagaacatttttattgaaatacaacgaaaacggaactataactgacgctgttgattgtgcacttgatccattaagtgagactaaatgtactctaaaaagttttactgttgaaaagggaatttatcaaacatcaaattttcgcgttcaaccaacggaaagtattgtacgttttccgaacataaccaatttatgtcctttcggtgaggtatttaacgcaactcgttttgcgagcgtatatgcttggaatagaaaaagaattagcaattgtgttgctgattattcggtcttatacaatagtgcttcgtttagcacttttaaatgttacggagtaagtccaacaaagttaaatgatctatgtttcactaatgtgtatgctgattcttttgttattagaggtgatgaagttcgacaaattgctccaggtcaaactggcaaaattgcggactataattataagctacctgatgattttactggctgtgtgattgcatggaatagtaataatctagattcgaaagtcggtgggaattataattatctttatagactatttagaaaatctaatttgaaaccatttgagagagatatatcaacagaaatttaccaggctggcagcacaccttgcaacggcgtagaaggttttaattgttattttccactacaaagttatggttttcaaccaactaatggcgtcgggtatcaaccatatagagttgtcgtactttcctttgaattacttcatgcaccagctaccgtttgtgggccaaagaaatcaactaatcttgtaaagaataaatgcgtcaattttaattttaatggccttacaggcactggagttttaacagaatccaataaaaaatttttaccttttcagcaatttggtagagatatagctgatactactgatgctgtaagagatcctcaaactctagagattttagatattaccccgtgttcatttggaggcgtaagcgttataactccaggcacgaacacatcaaatcaagttgctgtactatatcaagatgttaattgcacagaagtgcctgttgccattcatgcagatcaacttactcctacatggcgtgtatattctaccggatcaaatgtatttcagactagagctggttgtttaataggcgcagaacatgtaaataatagttatgagtgtgatataccaattggtgcaggaatatgtgcatcatatcagacacagacaaatagtcctcgtcgcgcaaga

SEQ ID NO: 7 - DNA, 8067 bp, Artificial Sequence
  Description of Artificial Sequence Synthetic polynucleotide
tcagtagcatcacaatcgattatagcttatacaatgtctttaggtgcggaaaatagtgtggcttattctaataattctatcgcaatccctaccaatttcactataagtgttacaaccgaaatcttaccagttagtatgacaaagacaagtgttgattgtactatgtatatatgtggcgattctactgagtgttctaatctcttattacaatatggttcgttttgtactcagttaaatcgagctcttacaggtatagctgtcgagcaagataagaatacccaggaagtctttgcacaggttaaacaaatttataaaactccaccaatcaaagattttggtgggtttaacttttctcaaatactacctgatccatctaaaccctctaaacgtagttttattgaagatttactttttaataaagtaactctagctgatgctggtttcattaaacaatacggcgattgtttgggtgatatagcggcacgtgatttaatatgcgcacagaaattcaacggtctgacagtcctacctccattattgacagatgaaatgattgctcaatatacatcagcattgcttgctggcactatcacgagtggatggacttttggtgctggcgctgctttacaaattccatttgccatgcaaatggcttatagatttaatggtattggtgttacacaaaatgttttatatgagaatcaaaagttaatagctaaccaatttaactctgcaattggcaagattcaggattcattatctagtacagcgagtgctttaggtaaactacaagatgtagtgaatcagaatgctcaagcactcaatactttggttaaacaattaagttcaaattttggtgcaatttcaagtgtactaaatgatattctaagtcgcttagatcctccagaggctgaagtacaaatcgatagactaattacaggtagattacagtcattacaaacttatgttactcaacagttaattagagctgcagaaataagagcatctgcaaatttggcagccactaagatgagtgagtgtgtccttggacaatcaaaacgtgtagatttttgcggaaagggatatcacttaatgtcatttccgcaatctgcacctcatggtgtcgtgtttcttcatgttacttacgttccggctcaagagaaaaacttcactacggctccagcgatttgtcatgatggtaaagctcattttcctcgtgagggtgtatttgtatcaaatggaacacattggtttgttactcaaagaaatttttatgagccacaaataataactacagataatacttttgttagcggtaactgtgacgtagttataggaatcgtaaacaacacagtgtatgatccattacaaccagagttagattcttttaaagaagaacttgataagtatttcaaaaatcatactagccctgatgttgaccttggtgacatatcaggcataaatgcatcagttgttaatattcaaaaagaaatagataggcttaatgaagttgctaaaaatcttaatgaatctttaatagatctacaagaacttggaaaatacgaacaatatataaaatggccttggtatatatggttagggtttattgctggtcttattgctattgtaatggtaactattatgctatgttgtatgacatcatgctgtagctgtctaaagggttgttgtagttgtggttcatgttgcaaatttgatgaagatgatagtgagccagttcttaaaggtgtaaaattgcattacacatgaggttaaggatccactagctcgtttcaaattaccgatgatatcggaccgttccaacttaccgaccagttcggcaggtatgtatttgcgtgcattcctatccaaaaaaacatcaagccaaaagcttgaaaaaacttacaacacagctcaacagagctagattgtaaaaccctgctttgttaagcagaacgcaaaaattgaatgacttatagtcatatcgcttcgaccctcgtagattagtagccttgagctattaactggttgaaacacttaccaaataaagattaaaagcgataaaaatgaaagataaagcagctaaaaacagagattttagaaagactattttatcagtgttacaacgcaataaagatggctcttttgctacgcaagcaaatagaaagtctattctgttgcaggcaactaaagaccttaaaaaggtagggtttagcaaggttacagccgaaaacttcggtaataagcattgctatgcacttagagaccattggagagcccaaggattagctacagcaacgataaaaaatcgtttagcttgtctaaggtggttaggcgagaaaatgggcaaagaactacccgataatcgaaaattagagattgagaacaggaagtatagcgataattcaatcaataaagcccaagaaatcgattttaaggcgatttctgccttaactgataggcaagccctagcaatacaattacagcgcgaatttgggcttcgtagagaagaaagtttgaagtttcagcctagttatgcaatcaaagagcataaaatcgagcttaaaagctcttggacaaagggtggaagaccacgagaaatcccaattttgaatgaaaaacagagagaattgttagaaaaagtaaaagaggtagcaggtaaaggctctctaattgagagcgaaaagtcttataagcaagcaatggaacatttcacgactcgctgtcaaagagcagggattaagaatgttcatggctttagacatgcgtatgctcaagatagatataggcaattaacagggcgtgagtgtcctaaaaatggtggattaacatctaagcagctaacacctgagcaaaagcaacaagactatgaagctagaatgactattagtgaagagttaggtcatggtagagaagatgtaacagtcaactacttaggcagataaaaagcaatatagctatagaagaaaagaaagctattttacatagtagatcgactcttcttagggattttatattttttgataaatcatctattttgctagttaaatcatcaaatttatcatcttgttgtttgactaaatctaagaatctattctcttttttaaaatcgttcatgcaaaccgcctatagctttcttctttttctgaaattatttgtcttcacaccataattaaattcccatttttataagtaaagtcttttaaaagcttgtcagtctcttctctagaaatgtaccaaattttacctattttaggatacttttcatgaagttcttctatttttccccagtcctttaatagtctacctttagagtctcgtaaatagttatctttgtgacaggggcctcttttatcttttttaatgtaactatatgttattccaacgtcactattactattatccaaatcttttttagcatgccagtaagaactttcataacttaactctatctttcgacctctttgatatacaacaataaagctatagccagtagtaacaacctgttttacttttgttaaatctattaacttcttatttatttttttatgtttttttgaaaatttaaatatttctatattcattcctacacttcctcaaatccaaatggtagcttatgattctcttctggtttcttttctaatttttttatatttgcaataaaaactctttttctatctttgatttttttattgtcccaattcctccaagtatcatcacaaaccctttcaatatcatgtaaatgatgatgtctaaatattgatctgacataatacagatctaggtctagttcatcacttaacacaacttctctaagtctttcagatgcttcgattggtatgtaatcctctttatttttagtatctaaaagcttttgcttaaattcttcttctgtctctgctaccttactaactgtaaacttgatatttgtaatcttacgaccatgttttctgtgatgatccttgtcatcataggttacaaaaatatccgataattgattaatctcttctagtgctggtaataggaacttatttttaaaatttgaatatctgttgctgtaacttttaggtaaatcaaaatcattaatcatatcatcgacatacaatacgcaatcaactatattagcataccctgcttgttcgcctaatttgcttttgagaagtaagtataatctgcttgaatacttacttttaaatgaaaatagtaactgtctttctgctttagtaaagtactcttgtagttgtatcatgtgtggcattaatgaccaatgaaactcgcaaattaaagcactgcttttagggtctgcttcaatatatgcaaaccagttagctatcttcgtttgttctttattcagccatactggcttagacattattgagtgcattaattgcttcaatctcactctgttatgcttaacccctgtagctttttcaagatcagataggcttatcttatacctgtgaaactctttatcttctcttttaaccattgaggcaactaagaatattaagttttgttcttcttttgtaaggctatactttcctgcaacaagagtattagacatagctatttctttgccagcatttacatttttaacttctttcatagaactagagtcattatctcgatatacaaattctataaaacttctattagtaaaacaactacttcataaaaaaaagtagttttaacgatacaaaaagtagttttaaattcaaaaagtgatacaaaaagtagttttaaattcaaaaagtgatacaaaaagtagttttaaattttttaaaaaagtgcttcaaagccttatgtagcaatacttacagaggattaaaaaaaaatctgacaatatataaagagaatatataaagagaatatcttaggggattttaaaaaaatcccacagactcaaagacttttttgactttttaaatcctagaaactatactttaagtacttatttaagtacatggatttagattatgcaaaccgttaattattcaacttttagaaatgaactatctgattcaatggatagagtaacaaaaaatcatagtcctatgattgtaactagaggttcaaaaaaagaagcagttgttatgatgtcgttagaggattcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaattcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagaacatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagaacttggcggcgaatgggctgaccgcttcctcgtgctttacggtatcgccgctcccgattcgcagcgcatcgccttctatcgccttcttgacgagttcttctgaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaaagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttggtacctggttactattgccatcatcacaatattaaaattaattttcttcatttatttttcttaaatattattattaaaaatagtaaatttaacttatctaaaaatagcataatatcatttttattaaaatatctaggttgaattcttagatattttgatatataattagatactaaattgataacttataaagaattaaattttcttttgtatgctaacttgattgctaatatgaattatactagttagtatgttgattataatgattagagttttaaataatggaggtaacaataggaggtacgtaatg

SEQ ID NO: 8 - DNA, 8400 bp, Artificial Sequence
  Description of Artificial Sequence Synthetic polynucleotide
ggtacctggttactattgccatcatcacaatattaaaattaattttcttcatttatttttcttaaatattattattaaaaatagtaaatttaacttatctaaaaatagcataatatcatttttattaaaatatctaggttgaattcttagatattttgatatataattagatactaaattgataacttataaagaattaaattttcttttgtatgctaacttgattgctaatatgaattatactagttagtatgttgattataatgattagagttttaaataatggaggtaacaataggaggtacgtaatggattataaagatcacgatggtgattacaaagaccatgatatagattataaggatgacgatgataagcatcatcatcaccaccatcatcatggaggtggttcaatgtctgtagcgagtcaatcaataatagcatatacaatgtcattaggcgcagaaaatagtgtggcttattctaataattctatcgcaatccctaccaatttcactataagtgttacaaccgaaatcttaccagttagtatgacaaagacaagtgttgattgtactatgtatatatgtggcgattctactgagtgttctaatctcttattacaatatggttcgttttgtactcagttaaatcgagctcttacaggtatagctgtcgagcaagataagaatacccaggaagtctttgcacaggttaaacaaatttataaaactccaccaatcaaagattttggtgggtttaacttttctcaaatactacctgatccatctaaaccctctaaacgtagttttattgaagatttactttttaataaagtaactctagctgatgctggtttcattaaacaatacggcgattgtttgggtgatatagcggcacgtgatttaatatgcgcacagaaattcaacggtctgacagtcctacctccattattgacagatgaaatgattgctcaatatacatcagcattgcttgctggcactatcacgagtggatggacttttggtgctggcgctgctttacaaattccatttgccatgcaaatggcttatagatttaatggtattggtgttacacaaaatgttttatatgagaatcaaaagttaatagctaaccaatttaactctgcaattggcaagattcaggattcattatctagtacagcgagtgctttaggtaaactacaagatgtagtgaatcagaatgctcaagcactcaatactttggttaaacaattaagttcaaattttggtgcaatttcaagtgtactaaatgatattctaagtcgcttagataaagttgaggctgaagtacaaatcgatagactaattacaggtagattacagtcattacaaacttatgttactcaacagttaattagagctgcagaaataagagcatctgcaaatttggcagccactaagatgagtgagtgtgtccttggacaatcaaaacgtgtagatttttgcggaaagggatatcacttaatgtcatttccgcaatctgcacctcatggtgtcgtgtttcttcatgttacttacgttccggctcaagagaaaaacttcactacggctccagcgatttgtcatgatggtaaagctcattttcctcgtgagggtgtatttgtatcaaatggaacacattggtttgttactcaaagaaatttttatgagccacaaataataactacagataatacttttgttagcggtaactgtgacgtagttataggaatcgtaaacaacacagtgtatgatccattacaaccagagttagattcttttaaagaagaacttgataagtatttcaaaaatcatactagccctgatgttgaccttggtgacatatcaggcataaatgcatcagttgttaatattcaaaaagaaatagataggcttaatgaagttgctaaaaatcttaatgaatctttaatagatctacaagaacttggaaaatacgaacaatatataaaatggccttggtatatatggttagggtttattgctggtcttattgctattgtaatggtaactattatgctatgttgtatgacatcatgctgtagctgtctaaagggttgttgtagttgtggttcatgttgcaaatttgatgaagatgatagtgagccagttcttaaaggtgtaaaattggggggatctggaatgtacagctttgtgtcagaagaaaccggtacactaattgttaatagcgttttactttttctggcttttgttgtgtttcttctagtaacattggccatcttgactgcactaagactttgtgcttattgctgtaatattgttaatgtttcattagtaaaacctagcttttatgtttattcgagagtcaaaaacctaaattccagtagagtacctgatttattagtataaggttaaggatccactagctcgtttcaaattaccgatgatatcggaccgttccaacttaccgaccagttcggcaggtatgtatttgcgtgcattcctatccaaaaaaacatcaagccaaaagcttgaaaaaacttacaacacagctcaacagagctagattgtaaaaccctgctttgttaagcagaacgcaaaaattgaatgacttatagtcatatcgcttcgaccctcgtagattagtagccttgagctattaactggttgaaacacttaccaaataaagattaaaagcgataaaaatgaaagataaagcagctaaaaacagagattttagaaagactattttatcagtgttacaacgcaataaagatggctcttttgctacgcaagcaaatagaaagtctattctgttgcaggcaactaaagaccttaaaaaggtagggtttagcaaggttacagccgaaaacttcggtaataagcattgctatgcacttagagaccattggagagcccaaggattagctacagcaacgataaaaaatcgtttagcttgtctaaggtggttaggcgagaaaatgggcaaagaactacccgataatcgaaaattagagattgagaacaggaagtatagcgataattcaatcaataaagcccaagaaatcgattttaaggcgatttctgccttaactgataggcaagccctagcaatacaattacagcgcgaatttgggcttcgtagagaagaaagtttgaagtttcagcctagttatgcaatcaaagagcataaaatcgagcttaaaagctcttggacaaagggtggaagaccacgagaaatcccaattttgaatgaaaaacagagagaattgttagaaaaagtaaaagaggtagcaggtaaaggctctctaattgagagcgaaaagtcttataagcaagcaatggaacatttcacgactcgctgtcaaagagcagggattaagaatgttcatggctttagacatgcgtatgctcaagatagatataggcaattaacagggcgtgagtgtcctaaaaatggtggattaacatctaagcagctaacacctgagcaaaagcaacaagactatgaagctagaatgactattagtgaagagttaggtcatggtagagaagatgtaacagtcaactacttaggcagataaaaagcaatatagctatagaagaaaagaaagctattttacatagtagatcgactcttcttagggattttatattttttgataaatcatctattttgctagttaaatcatcaaatttatcatcttgttgtttgactaaatctaagaatctattctcttttttaaaatcgttcatgcaaaccgcctatagctttcttctttttctgaaattatttgtcttcacaccataattaaattcccatttttataagtaaagtcttttaaaagcttgtcagtctcttctctagaaatgtaccaaattttacctattttaggatacttttcatgaagttcttctatttttccccagtcctttaatagtctacctttagagtctcgtaaatagttatctttgtgacaggggcctcttttatcttttttaatgtaactatatgttattccaacgtcactattactattatccaaatcttttttagcatgccagtaagaactttcataacttaactctatctttcgacctctttgatatacaacaataaagctatagccagtagtaacaacctgttttacttttgttaaatctattaacttcttatttatttttttatgtttttttgaaaatttaaatatttctatattcattcctacacttcctcaaatccaaatggtagcttatgattctcttctggtttcttttctaatttttttatatttgcaataaaaactctttttctatctttgatttttttattgtcccaattcctccaagtatcatcacaaaccctttcaatatcatgtaaatgatgatgtctaaatattgatctgacataatacagatctaggtctagttcatcacttaacacaacttctctaagtctttcagatgcttcgattggtatgtaatcctctttatttttagtatctaaaagcttttgcttaaattcttcttctgtctctgctaccttactaactgtaaacttgatatttgtaatcttacgaccatgttttctgtgatgatccttgtcatcataggttacaaaaatatccgataattgattaatctcttctagtgctggtaataggaacttatttttaaaatttgaatatctgttgctgtaacttttaggtaaatcaaaatcattaatcatatcatcgacatacaatacgcaatcaactatattagcataccctgcttgttcgcctaatttgcttttgagaagtaagtataatctgcttgaatacttacttttaaatgaaaatagtaactgtctttctgctttagtaaagtactcttgtagttgtatcatgtgtggcattaatgaccaatgaaactcgcaaattaaagcactgcttttagggtctgcttcaatatatgcaaaccagttagctatcttcgtttgttctttattcagccatactggcttagacattattgagtgcattaattgcttcaatctcactctgttatgcttaacccctgtagctttttcaagatcagataggcttatcttatacctgtgaaactctttatcttctcttttaaccattgaggcaactaagaatattaagttttgttcttcttttgtaaggctatactttcctgcaacaagagtattagacatagctatttctttgccagcatttacatttttaacttctttcatagaactagagtcattatctcgatatacaaattctataaaacttctattagtaaaacaactacttcataaaaaaaagtagttttaacgatacaaaaagtagttttaaattcaaaaagtgatacaaaaagtagttttaaattcaaaaagtgatacaaaaagtagttttaaattttttaaaaaagtgcttcaaagccttatgtagcaatacttacagaggattaaaaaaaaatctgacaatatataaagagaatatataaagagaatatcttaggggattttaaaaaaatcccacagactcaaagacttttttgactttttaaatcctagaaactatactttaagtacttatttaagtacatggatttagattatgcaaaccgttaattattcaacttttagaaatgaactatctgattcaatggatagagtaacaaaaaatcatagtcctatgattgtaactagaggttcaaaaaaagaagcagttgttatgatgtcgttagaggattcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaattcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagaacatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagaacttggcggcgaatgggctgaccgcttcctcgtgctttacggtatcgccgctcccgattcgcagcgcatcgccttctatcgccttcttgacgagttcttctgaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaaagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctt

SEQ ID NO: 9 - DNA, 10029 bp, Artificial Sequence
  Description of Artificial Sequence Synthetic polynucleotide
tgaggttaaggatccactagctcgtttcaaattaccgatgatatcggaccgttccaacttaccgaccagttcggcaggtatgtatttgcgtgcattcctatccaaaaaaacatcaagccaaaagcttgaaaaaacttacaacacagctcaacagagctagattgtaaaaccctgctttgttaagcagaacgcaaaaattgaatgacttatagtcatatcgcttcgaccctcgtagattagtagccttgagctattaactggttgaaacacttaccaaataaagattaaaagcgataaaaatgaaagataaagcagctaaaaacagagattttagaaagactattttatcagtgttacaacgcaataaagatggctcttttgctacgcaagcaaatagaaagtctattctgttgcaggcaactaaagaccttaaaaaggtagggtttagcaaggttacagccgaaaacttcggtaataagcattgctatgcacttagagaccattggagagcccaaggattagctacagcaacgataaaaaatcgtttagcttgtctaaggtggttaggcgagaaaatgggcaaagaactacccgataatcgaaaattagagattgagaacaggaagtatagcgataattcaatcaataaagcccaagaaatcgattttaaggcgatttctgccttaactgataggcaagccctagcaatacaattacagcgcgaatttgggcttcgtagagaagaaagtttgaagtttcagcctagttatgcaatcaaagagcataaaatcgagcttaaaagctcttggacaaagggtggaagaccacgagaaatcccaattttgaatgaaaaacagagagaattgttagaaaaagtaaaagaggtagcaggtaaaggctctctaattgagagcgaaaagtcttataagcaagcaatggaacatttcacgactcgctgtcaaagagcagggattaagaatgttcatggctttagacatgcgtatgctcaagatagatataggcaattaacagggcgtgagtgtcctaaaaatggtggattaacatctaagcagctaacacctgagcaaaagcaacaagactatgaagctagaatgactattagtgaagagttaggtcatggtagagaagatgtaacagtcaactacttaggcagataaaaagcaatatagctatagaagaaaagaaagctattttacatagtagatcgactcttcttagggattttatattttttgataaatcatctattttgctagttaaatcatcaaatttatcatcttgttgtttgactaaatctaagaatctattctcttttttaaaatcgttcatgcaaaccgcctatagctttcttctttttctgaaattatttgtcttcacaccataattaaattcccatttttataagtaaagtcttttaaaagcttgtcagtctcttctctagaaatgtaccaaattttacctattttaggatacttttcatgaagttcttctatttttccccagtcctttaatagtctacctttagagtctcgtaaatagttatctttgtgacaggggcctcttttatcttttttaatgtaactatatgttattccaacgtcactattactattatccaaatcttttttagcatgccagtaagaactttcataacttaactctatctttcgacctctttgatatacaacaataaagctatagccagtagtaacaacctgttttacttttgttaaatctattaacttcttatttatttttttatgtttttttgaaaatttaaatatttctatattcattcctacacttcctcaaatccaaatggtagcttatgattctcttctggtttcttttctaatttttttatatttgcaataaaaactctttttctatctttgatttttttattgtcccaattcctccaagtatcatcacaaaccctttcaatatcatgtaaatgatgatgtctaaatattgatctgacataatacagatctaggtctagttcatcacttaacacaacttctctaagtctttcagatgcttcgattggtatgtaatcctctttatttttagtatctaaaagcttttgcttaaattcttcttctgtctctgctaccttactaactgtaaacttgatatttgtaatcttacgaccatgttttctgtgatgatccttgtcatcataggttacaaaaatatccgataattgattaatctcttctagtgctggtaataggaacttatttttaaaatttgaatatctgttgctgtaacttttaggtaaatcaaaatcattaatcatatcatcgacatacaatacgcaatcaactatattagcataccctgcttgttcgcctaatttgcttttgagaagtaagtataatctgcttgaatacttacttttaaatgaaaatagtaactgtctttctgctttagtaaagtactcttgtagttgtatcatgtgtggcattaatgaccaatgaaactcgcaaattaaagcactgcttttagggtctgcttcaatatatgcaaaccagttagctatcttcgtttgttctttattcagccatactggcttagacattattgagtgcattaattgcttcaatctcactctgttatgcttaacccctgtagctttttcaagatcagataggcttatcttatacctgtgaaactctttatcttctcttttaaccattgaggcaactaagaatattaagttttgttcttcttttgtaaggctatactttcctgcaacaagagtattagacatagctatttctttgccagcatttacatttttaacttctttcatagaactagagtcattatctcgatatacaaattctataaaacttctattagtaaaacaactacttcataaaaaaaagtagttttaacgatacaaaaagtagttttaaattcaaaaagtgatacaaaaagtagttttaaattcaaaaagtgatacaaaaagtagttttaaattttttaaaaaagtgcttcaaagccttatgtagcaatacttacagaggattaaaaaaaaatctgacaatatataaagagaatatataaagagaatatcttaggggattttaaaaaaatcccacagactcaaagacttttttgactttttaaatcctagaaactatactttaagtacttatttaagtacatggatttagattatgcaaaccgttaattattcaacttttagaaatgaactatctgattcaatggatagagtaacaaaaaatcatagtcctatgattgtaactagaggttcaaaaaaagaagcagttgttatgatgtcgttagaggattcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaattcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagaacatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagaacttggcggcgaatgggctgaccgcttcctcgtgctttacggtatcgccgctcccgattcgcagcgcatcgccttctatcgccttcttgacgagttcttctgaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaaagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttggtacctggttactattgccatcatcacaatattaaaattaattttcttcatttatttttcttaaatattattattaaaaatagtaaatttaacttatctaaaaatagcataatatcatttttattaaaatatctaggttgaattcttagatattttgatatataattagatactaaattgataacttataaagaattaaattttcttttgtatgctaacttgattgctaatatgaattatactagttagtatgttgattataatgattagagttttaaataatggaggtaacaataggaggtacgtaatggattataaagatcacgatggtgattacaaagaccatgatatagattataaggatgacgatgataagcatcatcatcaccaccatcatcatggaggtggttcaatgtttgtgtttttagttcttttaccgttagtttcaagtcaatgtgtgaacttaactacacgcacacaacttcctccagcatatacaaatagttttactagaggtgtatattatcctgataaagtattccgtagttctgttctacattctacacaagatttgtttttaccgtttttcagtaatgtcacttggttccatgctattcatgtttctgggacaaacggtacaaaaagatttgataaccctgttttaccatttaatgatggtgtatattttgcttcaactgagaaaagcaatataattagaggttggattttcggaactaccctggatagcaagacgcaaagtttattgatcgtaaacaatgctacaaacgtcgtaattaaagtatgtgaatttcaattttgtaatgacccttttttaggagtctattatcataaaaataataaatcttggatggagtctgaatttagagtttattctagcgctaataactgtacatttgaatatgtttcacaaccttttttaatggatctagaaggtaaacagggtaattttaaaaatcttcgtgagtttgtttttaagaacatagatggatatttcaaaatatattcaaaacatactcctattaatctagttagagatcttccacaaggcttttctgctctagaaccattagttgatttaccaataggtataaatataactcgtttccaaactttactagcccttcaccgttcgtacttaacgcctggggattcttctagtggttggactgctggcgctgcagcatattatgttggatatctacaacctagaacatttttattgaaatacaacgaaaacggaactataactgacgctgttgattgtgcacttgatccattaagtgagactaaatgtactctaaaaagttttactgttgaaaagggaatttatcaaacatcaaattttcgcgttcaaccaacggaaagtattgtacgttttccgaacataaccaatttatgtcctttcggtgaggtatttaacgcaactcgttttgcgagcgtatatgcttggaatagaaaaagaattagcaattgtgttgctgattattcggtcttatacaatagtgcttcgtttagcacttttaaatgttacggagtaagtccaacaaagttaaatgatctatgtttcactaatgtgtatgctgattcttttgttattagaggtgatgaagttcgacaaattgctccaggtcaaactggcaaaattgcggactataattataagctacctgatgattttactggctgtgtgattgcatggaatagtaataatctagattcgaaagtcggtgggaattataattatctttatagactatttagaaaatctaatttgaaaccatttgagagagatatatcaacagaaatttaccaggctggcagcacaccttgcaacggcgtagaaggttttaattgttattttccactacaaagttatggttttcaaccaactaatggcgtcgggtatcaaccatatagagttgtcgtactttcctttgaattacttcatgcaccagctaccgtttgtgggccaaagaaatcaactaatcttgtaaagaataaatgcgtcaattttaattttaatggccttacaggcactggagttttaacagaatccaataaaaaatttttaccttttcagcaatttggtagagatatagctgatactactgatgctgtaagagatcctcaaactctagagattttagatattaccccgtgttcatttggaggcgtaagcgttataactccaggcacgaacacatcaaatcaagttgctgtactatatcaagatgttaattgcacagaagtgcctgttgccattcatgcagatcaacttactcctacatggcgtgtatattctaccggatcaaatgtatttcagactagagctggttgtttaataggcgcagaacatgtaaataatagttatgagtgtgatataccaattggtgcaggaatatgtgcatcatatcagacacagacaaatagtcctcgtcgcgcaagatcagtagcatcacaatcgattatagcttatacaatgtctttaggtgcggaaaatagtgtggcttattctaataattctatcgcaatccctaccaatttcactataagtgttacaaccgaaatcttaccagttagtatgacaaagacaagtgttgattgtactatgtatatatgtggcgattctactgagtgttctaatctcttattacaatatggttcgttttgtactcagttaaatcgagctcttacaggtatagctgtcgagcaagataagaatacccaggaagtctttgcacaggttaaacaaatttataaaactccaccaatcaaagattttggtgggtttaacttttctcaaatactacctgatccatctaaaccctctaaacgtagttttattgaagatttactttttaataaagtaactctagctgatgctggtttcattaaacaatacggcgattgtttgggtgatatagcggcacgtgatttaatatgcgcacagaaattcaacggtctgacagtcctacctccattattgacagatgaaatgattgctcaatatacatcagcattgcttgctggcactatcacgagtggatggacttttggtgctggcgctgctttacaaattccatttgccatgcaaatggcttatagatttaatggtattggtgttacacaaaatgttttatatgagaatcaaaagttaatagctaaccaatttaactctgcaattggcaagattcaggattcattatctagtacagcgagtgctttaggtaaactacaagatgtagtgaatcagaatgctcaagcactcaatactttggttaaacaattaagttcaaattttggtgcaatttcaagtgtactaaatgatattctaagtcgcttagatcctccagaggctgaagtacaaatcgatagactaattacaggtagattacagtcattacaaacttatgttactcaacagttaattagagctgcagaaataagagcatctgcaaatttggcagccactaagatgagtgagtgtgtccttggacaatcaaaacgtgtagatttttgcggaaagggatatcacttaatgtcatttccgcaatctgcacctcatggtgtcgtgtttcttcatgttacttacgttccggctcaagagaaaaacttcactacggctccagcgatttgtcatgatggtaaagctcattttcctcgtgagggtgtatttgtatcaaatggaacacattggtttgttactcaaagaaatttttatgagccacaaataataactacagataatacttttgttagcggtaactgtgacgtagttataggaatcgtaaacaacacagtgtatgatccattacaaccagagttagattcttttaaagaagaacttgataagtatttcaaaaatcatactagccctgatgttgaccttggtgacatatcaggcataaatgcatcagttgttaatattcaaaaagaaatagataggcttaatgaagttgctaaaaatcttaatgaatctttaatagatctacaagaacttggaaaatacgaacaa

SEQ ID NO: 10 - protein, 4 aa, Artificial Sequence
  Description of Artificial Sequence Synthetic peptide
GGSG

(19) United States
(12) Patent Application Publication
(10) Pub. No.: US 2023/0181720 A1
(43) Pub. Date: Jun. 15, 2023

(54) SAFE POTENT SINGLE VECTOR PLATFORM VACCINE AGAINST COVID-19

(71) Applicant: The Regents of the University of California, Oakland, CA (US)

(72) Inventors: Marcus A. Horwitz, Los Angeles, CA (US); Qingmei Jia, Los Angeles, CA (US)

(73) Assignee: The Regents of th University of California, Oakland, CA (US)

(21) Appl. No.: 17/924,304

(22) PCT Filed: May 13, 2021
(86) PCT No.: PCT/US21/32203

§ 371 Date: Nov. 9, 2022

Related U.S. Application Data
(60) Provisional application No. 63/182,111, filed on Apr. 30, 2021.
(60) Provisional application No. 63/026,480, filed on May 18, 2020.

(51) Int. Cl. A61K 39/215; A61P 31/14; C07K 14/165; C12N 1/20; C12N 15/62

(52) U.S. Cl. CPC A61K 39/215; A61P 31/14; C07K 14/165; C12N 1/20; C12N 15/62; A61K 2039/523

(57) ABSTRACT

Embodiments of the invention include immunogenic compositions that comprise an attenuated recombinant Francisella tularensis subspecies holarctica Live Vaccine Strain (LVS) having a deletion in a polynucleotide encoding CapB (LVS ΔcapB), wherein the LVS ΔcapB expresses one or more antigens present on severe acute respiratory syndrome coronavirus 2 (SARS-CoV-2), Embodiments of the invention also include methods of immunizing a susceptible host against a pathogen comprising administering to the host a vaccine that comprises an attenuated recombinant Live Vaccine Strain lacking a polynucleotide encoding CapB (LVS ΔcapB), wherein the LVS ΔcapB expresses one or more antigens expressed by a severe acute respiratory syndrome coronavirus 2 (SAR8-CoV-2) polypeptide.

### CROSS REFERENCE TO RELATED APPLICATIONS

This application claims the benefit under 35 U.S.C. Section 119(e) of co-pending and commonly-assigned U.S. Provisional Patent Application Ser. No. 63/026,480, filed on May 18, 2020, and U.S. Provisional Patent Application Ser. No. 63/182,111, filed on Apr. 30, 2021, which applications are incorporated by reference herein.

### STATEMENT OF GOVERNMENT INTEREST

This invention was made with Government support under grant number AI141390, awarded by the National Institutes of Health. The Government has certain rights in the invention.

### TECHNICAL FIELD

The invention relates to single platform vaccines for preventing diseases caused by pathogens and in particular, COVID-19.

### BACKGROUND OF THE INVENTION

Severe acute respiratory syndrome coronavirus 2 (SARS-CoV-2), closely related to SARS-CoV, is an enveloped, single-stranded positive RNA virus with a nucleocapsid that belongs to the betacoronavirus genus of the Coronaviridae. Starting in the final months of 2019, the virus caused an ongoing pandemic of COVID-19; the pandemic originated in Wuhan, Hubei Province of China and quickly spread worldwide with millions of confirmed cases and hundreds of thousands of fatalities.

The virus is primarily spread between people during close contact, most often via small droplets produced by coughing, sneezing, and talking. The droplets usually fall to the ground or onto surfaces rather than travelling through air over long distances. The time from exposure to onset of symptoms is typically around five days but may range from two to fourteen days. Common symptoms include fever, cough, fatigue, shortness of breath, and loss of smell and taste. While the majority of cases result in mild symptoms, some progress to acute respiratory distress syndrome (ARDS), multi-organ failure, septic shock, and blood clots.

There are currently no vaccines available to prevent COVID-19. Accordingly, there is a need for vaccines and associated methods designed to protect individuals from COVID-19 infection.

### SUMMARY OF THE INVENTION

The invention disclosed herein provides a SARS-CoV-2 vaccine vector platform which is useful for preventing the disease COVID-19 caused by SARS-CoV-2 in humans and animals. The invention utilizes a vector termed “LVS ΔcapB”, which is a live attenuated capB mutant of Francisella tularensis Live Vaccine Strain (LVS), itself attenuated by serial passage in the 20th century from Francisella tularensis subsp. holarctica. In this context, LVS has two major attenuating deletions and several minor mutations. The invention is also the use of this vaccine platform to construct and use vaccines against numerous other pathogens caused by bacteria, viruses, parasites, etc.

Embodiments of the invention include an immunogenic composition comprising at least one recombinant attenuated Francisella tularensis subspecies holarctica Live Vaccine Strain (LVS) having a deletion in a capB gene and an antigen expression cassette which comprises a F. tularensis promoter and which expresses at least one antigenic epitope present in a polypeptide expressed by severe acute respiratory syndrome coronavirus 2 (SARS-CoV-2). In such compositions, the antigenic polypeptide epitope elicits an immune response in a mammalian host when the immunogenic composition is administered orally (p.o.), intradermally (i.d.), subcutaneously (s.q.), intramuscularly (i.m.), intranasally (i.n.), or by inhalation to the mammalian host.

In typical embodiments of the invention, the at least one antigenic polypeptide epitope present in the polypeptide expressed by severe acute respiratory syndrome coronavirus 2 is present on: a SARS-CoV-2 large surface spike (S) glycoprotein; a SARS-CoV-2 envelope (E) protein: a SARS-CoV-2 membrane (M) glycoprotein: and/or a SARS-CoV-2 nucleocapsid (N) phosphoprotein. Optionally in these compositions, the polypeptide expressed by severe acute respiratory syndrome coronavirus 2 comprises at least two antigenic polypeptide epitopes present in: a SARS-CoV-2 large surface spike (S) glycoprotein: a SARS-CoV-2 envelope (E) protein; a SARS-CoV-2 membrane (M) glycoprotein: and/or a SARS-CoV-2 nucleocapsid (N) phosphoprotein.

In certain embodiments of the invention, the at least one antigenic polypeptide epitope present in the polypeptide expressed by severe acute respiratory syndrome coronavirus 2 is present on SARS-CoV-2 membrane (M) glycoprotein; or SARS-CoV-2 nucleocapsid (N) phosphoprotein. Typically, in these embodiments, the LVS ΔcapB expresses at least two antigenic polypeptide epitopes present on severe acute respiratory syndrome coronavirus 2 including: at least one peptide epitope present in SARS-CoV-2 membrane (M) glycoprotein; at least one peptide epitope present in SARS-CoV-2 nucleocapsid (N) phosphoprotein. In illustrative working embodiments of the invention disclosed herein, the at least two antigenic polypeptide epitopes present on a severe acute respiratory syndrome coronavirus 2 polypeptide are encoded by a sequence found in SEQ ID NO: 1 (e.g., a polynucleotide sequence encoding SARS-CoV-2 membrane (M) glycoprotein coupled via a polypeptide linker to a SARS-CoV-2 nucleocapsid (N) phosphoprotein). In these working embodiments, the antigenic polypeptide is encoded in a codon optimized polynucleotide sequence (i.e., one optimized for expression in Francisella tularensis).

Related embodiments of the invention method of making an immunogenic composition, such methods comprising introducing a polynucleotide encoding at least one antigenic epitope present in a polypeptide expressed by severe acute respiratory syndrome coronavirus 2 (SARS-CoV-2) into a recombinant attenuated Francisella tularensis subspecies holarctica Live Vaccine Strain (LVS). In these methods, the LVS has a deletion in a capB gene; and the antigenic polypeptide epitope encoded by the polynucleotide elicits an immune response to SARS-CoV-2 in a mammalian host when the immunogenic composition is administered orally (p.o.), intradermally (i.d.), subcutaneously (s.q.), intramuscularly (i.m.), intranasally (i.n.) or by inhalation to the mammalian host. Embodiments of the invention include making compositions of matter that further comprise additional agents such as a pharmaceutical excipient selected for a specific route of administration, for example oral or intranasal administration. In certain embodiments, the at least one antigenic polypeptide epitope present in the polypeptide expressed by severe acute respiratory syndrome coronavirus 2 is present on SARS-CoV-2 membrane (M) glycoprotein; or SARS-CoV-2 nucleocapsid (N) phosphoprotein. Typically, in these embodiments, the LVS ΔcapB expresses at least two antigenic polypeptide epitopes including: at least one peptide epitope present in SARS-CoV-2 membrane (M) glycoprotein; at least one peptide epitope present in SARS-CoV-2 nucleocapsid (N) phosphoprotein. In illustrative working embodiments of the invention that are disclosed herein, the at least two antigenic polypeptide epitopes present on a severe acute respiratory syndrome coronavirus 2 polypeptide arc encoded by SEQ ID NO: 1.

Other embodiments of the invention include the use of an immunogenic composition disclosed herein for inducing immunity to SARS-CoV-2. Such embodiments of the invention include methods of generating an immune response in a mammal comprising administering the immunogenic composition disclosed herein (e.g., a LVS ΔcapB transformed with a polynucleotide encoding a SARS-CoV-2 M and N fusion protein such as the polynucleotide of SEQ ID NO: 1) to the mammal so that an immune response is generated to the antigenic polypeptide epitope present in a severe acute respiratory syndrome coronavirus 2 polypeptide. In certain embodiments of the invention, the immunogenic composition is administered orally. In other embodiments of the invention, the immunogenic composition is administered intranasally.

Embodiments of the vaccine platform disclosed herein can be modified to accommodate mutated antigens of SARS-CoV-2 and future SARS-CoV-like viruses should such strains arise and be sufficiently different from SARS-CoV-2 that persons or animals vaccinated with an earlier vaccine version are no longer immune. The vaccine platform can be used to construct vaccines against other viruses including but not limited to SARS, MERS, and other coronaviruses: Influenza A and B: Hepatitis A. Hepatitis B, Hepatitis C, Hepatitis E; Ebolavirus; Lassa; Nipah; Rift Valley Fever; Zika; Chikungunya; Cocksackie A16; Enterovirus 68, Enterovirus 71; Marburg; HIV; Dengue; Rabies: Arenaviruses including Guanarito, Junin, Lassa, Lujo, Machupo, Sabia, Dandemong, lymphocytic choriomeningitis; Bunyaviruses including Andes, Bwamba, Crimean-Congo Hemorrhagic Fever, Oropouche, Rift Valley, Severe Fever with Thrombocytopenia, Syndrome (SFTS); Flaviviruses including Japanese encephalitis, Usutu, West Nile; Togaviruses including Bamah Forest, O'nyong-nyong, Ross River, Semliki Forest, Venezuelan Equine Encephalitis; Filviruses including Bundibugyo Ebola, Lake Victoria Marburg, Sudan Ebola: Herpesviruses: Polyomaviruses: Poxviruses, Cytomegalovirus, Epstein-Barr, etc. The vaccine platform can be used to construct vaccines against bacteria including but not limited to Burkholderia, pseudomallei, Burkholderia mallei, Francisella tularensis, Bacillus anthracis, Yersinia pestis, Mycobacterium tuberculosis, Mycobacterium leprae, Legionella pneumophila, Chlamydia trachomatis, Chlamydia pneumoniae, Chlamydia psittaci, Listeria monocytogenes, Brucella species, etc. The vaccine platform can be used to construct vaccines against rickettsia including but not limited to Rickettsia prowazekii, R. typhi, R. rickettsia, R. tsutsugamushi, Coxiella burnetii, etc. The vaccine platform can be used to construct vaccines against protozoa including but not limited to Leishmania species, Trypanosoma cruzi, Toxoplasma gondii, etc. The vaccine platform can be used to construct vaccines against fungi including but not limited to Histoplasma capsulatum, Coccidioides immitis or Coccidioides posadasii, etc.

As noted above, in certain embodiments of the invention, combinations of vaccines expressing different SARS-CoV-2 antigens can be administered together. The vaccine platform has consistently resulted in a strong antibody response and a strong cell-mediated immune response to recombinant pathogen antigens expressed by the vaccine. The vaccine composition is administered to humans or animals by injection intradermally or by another route, e.g., subcutaneously, intramuscularly, orally, intranasally, or by inhalation. Each vaccine composition can be administered intradermally (i.d.) or by another route, e.g., subcutaneously (s.q.), intramuscularly (i.m.), intranasally (i.n.), inhaled, or even orally (p.o.) to a mammalian host. The vaccine can be administered as part of a homologous or heterologous prime-boost vaccination strategy. In certain implementations, the host is administered a single dose of a first vaccine and one or more doses of a homologous or heterologous booster vaccine.

This single platform simplifies manufacture, regulatory approval, clinical evaluation, and vaccine administration, and would be more acceptable to people than multiple individual vaccines, and be less costly. Currently, no single bacterial platform vaccine against SARS-CoV-2 is available. Regarding manufacture, vaccines constructed from the same vectors can be manufactured under the same conditions. That is, the manufacture of the LVS ΔcapB vector will be the same regardless of which antigen it is expressing or overexpressing. Similarly, manufacture of the L. monocytogenes vector will be the same regardless of which antigen it is expressing.

Other objects, features and advantages of the present invention will become apparent to those skilled in the art from the following detailed description. It is to be understood, however, that the detailed description and specific examples, while indicating some embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

FIGS. 1A-1C. Schematics showing the construction of rLVS ΔcapB/SARS-CoV-2 vaccines. FIG. 1A. Schematic of SARS-CoV-2 genomic region encoding four major structural proteins, Spike (S), Envelope (E), Membrane (M), and Nucleocapsid (N) protein. FIG. 1B & FIG. 1C. Diagrams of S protein and the antigen expression cassettes. SP, signal peptide for S protein; NTD, N-terminal domain; RBD, receptor binding domain; FP, fusion peptide; HR, heptad repeat; CH, central helix: CD, central domain; and TM, Transmembrane domain (1); R, ribosome entry site: Pbfr, Ft bacterioferritin (FTT1441) promoter; Pomp, F. novicida omp (FTN_1451) promoter.

FIG. 2. Expression of SARS-CoV-2 MN and S2E proteins by rLVS ΔcapB vaccines. Total bacterial lysates of 4 clones (clones #1, 2, 3, 4) of rLVS ΔcapB/SCoV2-N3F-MN (lanes 4-7) and 4 clones (clone #1, 2, 3, 4) of rLVS ΔcapB/SCoV2-N3F-S2E (lanes 9-12) were analyzed by SDS-PAGE and Western blotting with an anti-FLAG monoclonal antibody (Top panel) and an anti-SARS-CoV-1 guinea pig polyclonal antibody (BEI Resources, NR-10361) (Bottom panel). Note that the N3F-MN (lanes 4-7) was readily detected by the pAb against SARS-CoV-1 (bottom panel) but not the mAb against FLAG (top panel); in contrast, the N3F-S2E protein (lanes 9-12) was readily detected by the mAb against FLAG (top panel) but poorly detected by the pAb against SARS-CoV-1 (bottom panel). The estimated molecular weights of the full-length N3F-MN and N3F-S2E are 75- and 77-kDa, respectively. The full-length N3F-MN protein (75-kDa) and the major breakdown product, the N protein (46 kDa), are indicated by blue color-coded asterisks to the right of the bands in the lower panel. The full-length N3F-S2E protein (77-kDa) is indicated by an orange color-coded asterisk to the right of the bands in the top panel. The protein bands of the positive control of SARS-CoV1 N (lane 14) and SΔTM protein (lane 15) are also indicated by green color-coded asterisks to the right of the bands. The size of the molecular weight markers (m) are labeled to the left of the panels. Top and bottom panels: pre-stained standards are visible (lanes 2 and 8): unstained standards are not visible on the Western blot (lane 1).

FIG. 3. Expression of SARS-CoV-2 Spike protein by LVS ΔcapB vaccines. Total bacterial lysates of LVS ΔcapB vector (lane 3), 3 clones (clones #1, 2, 3,) of rLVS ΔcapB/SCoV2-N3F-S (lanes 4-6), 3 clones (clone #1, 2, 3) of rLVS ΔcapB/SCoV2-S (lanes 7-9) and 3 clones (clone #1, 2, 3) of rLVS ΔcapB/SCoV2-Sc with a C-terminal tag (lanes 10-12) were analyzed by SDS-PAGE and Western blotting with an anti-FLAG monoclonal antibody (mAb) (Top panel) and an anti-SARS-CoV-1 guinea pig polyclonal antibody (pAb) (BEI Resources, NR-10361) (Bottom panel). Note that the N3F-S protein (lanes 4-6) was detected by both the mAb against FLAG (top panel) and the pAb against SARS-CoV-1 (bottom panel): the S with a C-terminal tag (Sc) (lanes 10-12) was not detected by the mAb against FLAG (top panel) but detected by the pAb against SARS-CoV-1 (bottom panel). SARS-CoV1 proteins of M (BET Resources, NR-878, ˜27 kDa) (lane 13), N (BEI Resources, NR-699, 46 kDa) (lane 14), and SΔTM (BEI Resources, NR-722, ˜150 kDa) (lane 15) served as positive controls. Both mAb against FLAG and pAb against SARS-CoV-1 detected multiple non-specific bands from the total lysates. The estimated molecular weight of the N3F-S is 143 kDa, as indicated by red color asterisks to the right of the protein bands in lanes 4-6 and lanes 10-12. The positive control of the SARS-CoV1 SΔTM is also indicated by a red asterisk (lane 15). The sizes of the molecular weight markers (m) are labeled to the left of the panels. Top and bottom panels: pre-stained standards are visible (lane 2); unstained standards are barely visible (lane 1).

FIG. 4. Expression of SARS-CoV-2 SΔTM, S1, and S2 subunit proteins by rLVS ΔcapB vaccines. Total bacterial lysates of LVS ΔcapB vector (lane 2), 4 clones (clones #1, 2, 7, 8) of rLVS ΔcapB/SCoV2-N3F-SΔTM (lanes 3-6), 4 clones (clone #1, 2, 6, 7) of rLVS ΔcapB/SCoV2-N3F-S1 (lanes 7-10) and 4 clones (clone #2, 6, 8, 12) of rLVS ΔcapB/SCoV2-S2 (lanes 11-14) were analyzed by SDS-PAGE and Western blotting with an anti-FLAG monoclonal antibody (Top panel) and an anti-SARS-CoV-1 guinea pig polyclonal antibody (BEI Resources, NR-10361) (Bottom panel). Note that the N3F-SΔTM protein (˜138 kDa) (lanes 3-6), indicated by a red asterisk to the right of the bands, was detected by both the mAb against FLAG (top panel) and the pAb against SARS-CoV-1 (bottom panel); the N3F-S1 (lanes 7-10) with two different molecular weights, indicated by purple asterisks to the right of the protein bands (top panel), were detected by the mAb against FLAG (top panel) but not detected by the pAb against SARS-CoV-1 (bottom panel); the un-tagged S2 (65 kDa) (lanes 11-14), indicated by a blue color-coded asterisk to the right of the protein bands (bottom panel), was detected by the pAb against SARS-CoV-1 (bottom panel). The SARS-CoV1 protein of SΔTM (BEI Resources. NR-722, ˜150 kDa) (lane 15), indicated by a green asterisk to the right of the protein band (lane 15) (bottom panel), served as a positive control. Top and bottom panels: Molecular weight standards are visible (lane 1) and the sizes of the molecular weight markers (m) are labeled to the left of the panels.

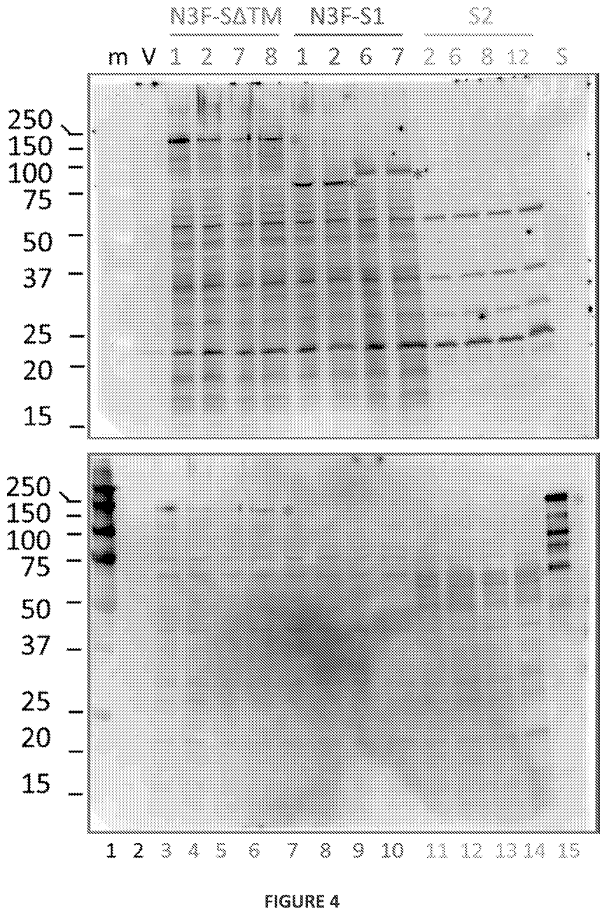

FIG. 5. Schematic of Francisella tularensis subspecies holarctica Live Vaccine Strain immunogenic compositions designed to express multiple SARS-CoV-2 proteins. As shown in this schematic, in certain embodiments of the invention, one or more SARS-CoV-2 proteins (e.g., the MN proteins) are disposed on the Francisella tularensis chromosome, while other SARS-CoV-2 proteins (e.g. the SΔTM (or S or S1 or S2), are disposed on a plasmid within this microorganism.

FIGS. 6a-b. Experimental schedule and weight loss after challenge, a Experiment schedule. FIG. 6a shows a schematic of an immunization schedule where Golden Syrian hamsters (8/group, equal sex) were immunized ID or IN twice (Week 0 and 3) with rLVS ΔcapB/SCoV2 vaccines, singly and in combination (MN+SΔTM; MN+S1); challenged IN 5 weeks later (Week 8) with 10⁵ pfu of SARS-CoV-2 (2019-nCoV/USA-WA1/2020 strain), and monitored closely for clinical signs of infection including weight loss. FIG. 6b shows graphed data from these studies. Single vaccines expressed the S, SΔTM, S1, S2, S2E, or MN proteins, as indicated. Control animals were sham-immunized (PBS) or immunized with the vector (LVS ΔcapB) only. All hamsters were assayed for oropharyngeal viral load at 1, 2, and 3 days post challenge (dpi). Half of the hamsters (n=4/group) were euthanized at 3 days post challenge for lung viral load analysis and half (n=4/group) were monitored for weight loss for 7 days and euthanized at 7 days post challenge for lung histopathology evaluation, b Weight loss after challenge. Data are mean % weight loss from 0 days post challenge. *P<0.05: **P≤0.01; ***P<0.001; ****P≤0.0001 comparing means on Day 7 post challenge by repeated measure (mixed) analysis of variance model. Sham vs. MN: P<0.0001, ID route; P<0.01, IN route. The standard errors were omitted in the graphs for clarity.

FIGS. 7a-7b. Lung histopathology on Day 7 after SARS-CoV-2 IN challenge. Hamsters (n=4, equal sex) were immunized ID or IN as described in FIG. 6 and euthanized at 7 days post challenge for histopathologic examination of their lungs. FIG. 7a shows data from studies of cranial and caudal lung histopathology post challenge in hamsters immunized ID (left) or IN (right); lungs were separately scored on a 0-5 or 0-4 scale for overall lesion extent, bronchitis, alveolitis, pneumocyte hyperplasia, vasculitis, and interstitial inflammation; the sum of the scores for each lung are shown (mean±SE). The histopathological score evaluation was performed by a single pathologist blinded to the identity of the groups. Each symbol represents one animal. Data are mean f SE. **P<0.01; ***P<0.001; ****P<0.0001 by two-way ANOVA with Tukey's multiple comparisons (GraphPad Prism 8.4.3): ns, not significant. FIG. 7b show data on the mean percentage reduction in the combined cranial and caudal lung histopathology score compared with Sham (PBS)-immunized animals calculated for each vaccine.

### DETAILED DESCRIPTION OF THE INVENTION

In the description of embodiments, reference may be made to the accompanying figures which form a part hereof, and in which is shown by way of illustration a specific embodiment in which the invention may be practiced. It is to be understood that other embodiments may be utilized, and structural changes may be made without departing from the scope of the present invention.

All publications mentioned herein are incorporated by reference to disclose and describe aspects, methods and/or materials in connection with the cited publications. Many of the techniques and procedures described or referenced herein are well understood and commonly employed by those skilled in the art. Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. This application is related to U.S. patent application Ser. No. 16/319,812, filed on Jan. 22, 2019, entitled “SAFE POTENT SINGLE PLATFORM VACCINE AGAINST TIER 1 SELECT AGENTS AND OTHER PATHOGENS” the contents of which are incorporated herein by reference.

The current pandemic of COVID-19 has sickened over a hundred and fifty million people, killed over 3 million, and wreaked havoc on the world's economy. There is a tremendous need for a safe and effective COVID-19 vaccine to end the current devastating pandemic. An effective COVID-19 vaccine can end this pandemic quickly.

The invention disclosed herein utilizes a vaccine vector platform termed “LVS ΔcapB”, which is a live attenuated capB mutant of Francisella tularensis Live Vaccine Strain (LVS), itself attenuated by serial passage in the 20th century from Francisella tularensis, subsp. holarctica (see, e.g., Jia et al., Infect Immun. 78:4341-4355. (Epub 2010 07-19). PMID 20643859. PMCID: PMC2950357. doi: 10.1128/IAI.00192-10; Salomonsson et al., Infect. Immun. 77:3424-343: and Rohmer et al., Infect. Immun. 74:6895-6906: the contents of which are incorporated herein by reference).

In this context, embodiments of the invention include immunogenic (vaccine) compositions that comprise an attenuated recombinant Francisella tularensis subspecies holarctica Live Vaccine Strain (LVS) that does not express CapB protein (e.g., LVS ΔcapB), wherein this LVS further expresses one or more antigens present on severe acute respiratory syndrome coronavirus 2 (SARS-CoV-2). Embodiments of the invention also include methods of immunizing a susceptible host against a pathogen comprising administering to the host a vaccine that comprises an attenuated recombinant Live Vaccine Strain lacking a polynucleotide encoding CapB (LVS ΔcapB), wherein the LVS ΔcapB expresses one or more antigens expressed by a severe acute respiratory syndrome coronavirus 2 (SARS-CoV-2) polypeptide.

One major advantage of the immunogenic vaccine compositions disclosed herein is the capacity to manufacture vaccines cheaply and quickly. The head of GAVI (the Vaccine Alliance) has pointed out how important it is that vaccines being developed for COVID-19 be available to all of the world's population and not just the privileged. The capacity to manufacture huge quantities of vaccine quickly and cheaply would allow that eventuality. Live, attenuated bacterial vaccines, such as LVS ΔcapB vectored vaccine against COVID-19 are much less expensive to manufacture, as they can be grown readily in inexpensive broth and require no purification. Vaccine cost is of critical importance in developing countries.

Another major advantage of the immunogenic vaccine compositions disclosed herein is that the vector is a more attenuated derivative of a vaccine already safely administered to people. Hence it is anticipated to be extremely safe. Another likely advantage of the immunogenic vaccine compositions disclosed herein is that as a live attenuated vaccine, it is much more likely to induce long-lasting protection than a protein/adjuvant vaccine, DNA/RNA vaccine, or non-replicating virus-vectored vaccine. Another major advantage of the immunogenic vaccine compositions disclosed herein is that the single vector platform that we are using is easily expandable to other infectious diseases. In fact, we have already employed the single platform to generate potent vaccine candidates against other pathogens. Finally, the immunogenic vaccine compositions disclosed herein is easily altered in response to mutations in the SARS-CoV-2 virus that may render initial vaccines against it no longer effective.

As there are currently no licensed vaccines against COVID-19 comprising a replicating bacterial vector, this vaccine meets a major unmet need. Previous human trials have demonstrated reasonable safety of the double-deletional parent vector (LVS). The even more attenuated but still highly immunogenic triple-deletional platform vector (LVS ΔcapB) derived from the parent is >10,000 fold less virulent in a mouse model (as measured by intranasal LD50; all animals survived highest dose tested). Because the vaccine is based upon a bacterial vector, it can be inexpensively manufactured in broth culture—no purification is necessary as in the case of viral-vectored vaccines.

Advantages of the invention disclosure herein include that there is no need for animal products, in contrast to viral-vectored vaccines grown in cell culture. In addition, there is no need for adjuvant; and the vaccine can be readily altered to accommodate mutations in the SARS-CoV-2 virus. In addition, single vector platform simplifies manufacture, regulatory approval, clinical evaluation, and vaccine administration, and would be more acceptable to people than multiple individual vaccines, and be less costly. Regarding manufacture, vaccines constructed from the same vectors can be manufactured under the same conditions. That is, the manufacture of the LVS ΔcapB vector will be the same regardless of which antigen it is expressing or overexpressing.

The invention disclosed herein has a number of embodiments. Embodiments of the invention include an immunogenic composition comprising at least one recombinant attenuated Francisella tularensis subspecies holaretica Live Vaccine Strain (LVS) having a deletion in a capB gene and an antigen expression cassette which comprises a F. tularensis promoter and which expresses at least one antigenic epitope present in a polypeptide expressed by severe acute respiratory syndrome coronavirus 2 (SARS-CoV-2). In such compositions, the antigenic polypeptide epitope elicits an immune response in a mammalian host when the immunogenic composition is administered by at least one route of administration selected from orally (p.o.), intradermally (i.d.), subcutaneously (s.q.), intramuscularly (i.m.), intranasally (i.n.), or by inhalation to the mammalian host.

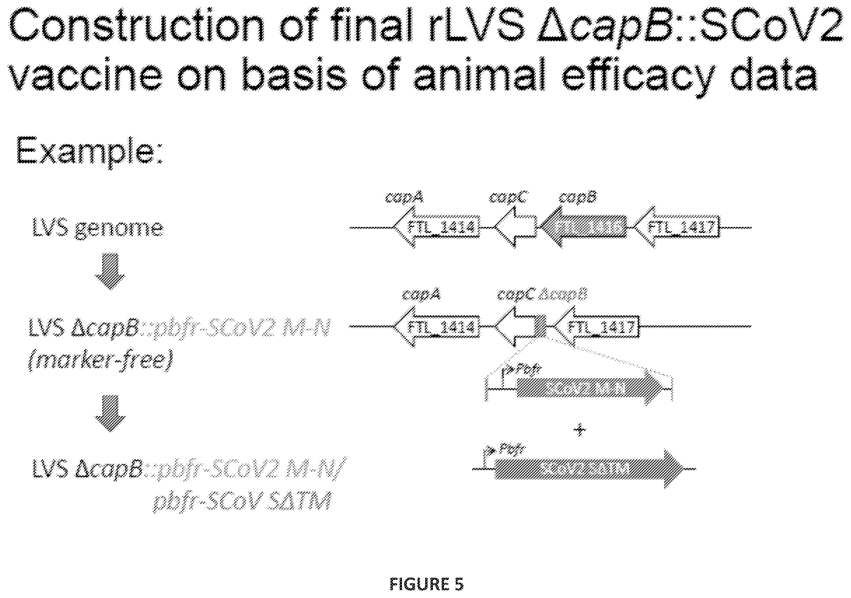

In typical embodiments of the invention, the at least one antigenic polypeptide epitope present in the polypeptide expressed by severe acute respiratory syndrome coronavirus 2 is present on: a SARS-CoV-2 large surface spike (S) glycoprotein; a SARS-CoV-2 envelope (E) protein: a SARS-CoV-2 membrane (M) glycoprotein: and/or a SARS-CoV-2 nucleocapsid (N) phosphoprotein. Optionally in these compositions, the polypeptide expressed by severe acute respiratory syndrome coronavirus 2 comprises at least two antigenic polypeptide epitopes present in: a SARS-CoV-2 large surface spike (S) glycoprotein: a SARS-CoV-2 envelope (E) protein; a SARS-CoV-2 membrane (M) glycoprotein: and/or a SARS-CoV-2 nucleocapsid (N) phosphoprotein (e.g. an epitope present on an S1 subunit of the SARS-CoV-2 large surface spike (S) glycoprotein and an epitope present on a S2 subunit of the SARS-CoV-2 large surface spike (S) glycoprotein). In certain embodiments of the invention, the antigenic polypeptide epitope is encoded in a codon optimized polynucleotide sequence. Optionally, the at least one antigenic epitope present in a polypeptide expressed by severe acute respiratory syndrome coronavirus 2 is encoded in a polynucleotide of SEQ ID NO: 1-SEQ ID NO: 9 (e.g. a polynucleotide segment in SEQ ID NO: 1, SEQ ID NO: 2, SEQ ID NO: 3, SEQ ID NO: 4, SEQ ID NO: 5. SEQ ID NO: 6, SEQ ID NO: 7, SEQ ID NO: 8 or SEQ ID NO: 9 that is at least 25, 50, 100, 200, 300, 400, 500, 1000, 2000, 3000, 4000, 5000, 6000, 7000 or 8000 nucleotides in length and/or is not more than 25, 50, 100, 200, 300, 400, 500, 1000, 2000, 3000, 4000, 5000, 6000, 7000 or 8000 nucleotides in length). Embodiments of the invention include Francisella tularensis subspecies holarctica Live Vaccine Strain immunogenic compositions that are designed to express multiple SARS-CoV-2 proteins from different genetic elements in this microorganism. For example, as shown in FIG. 5, in certain embodiments of the invention, one or more SARS-CoV-2 proteins (e.g. the MN proteins) are disposed on the Francisella tularensis chromosome, while other SARS-CoV-2 proteins (e.g. the SΔTM (or S or S1 or S2), are disposed on a plasmid within this microorganism.

In certain embodiments of the invention, the LVS is engineered to express at least two antigenic polypeptide epitopes present on severe acute respiratory syndrome coronavirus 2 including: at least one peptide epitope present in SARS-CoV-2 membrane (M) glycoprotein; at least one peptide epitope present in SARS-CoV-2 nucleocapsid (N) phosphoprotein. In certain embodiments of the invention, the LVS is transformed with a polynucleotide encoding polypeptide epitopes found on SARS-CoV-2 membrane (M)glycoprotein, with such polynucleotide sequences being coupled to a polynucleotide encoding a polypeptide linker, with this (encoded) linker also being coupled to a polynucleotide encoding polypeptide epitopes found on a SARS-CoV-2 nucleocapsid (N) phosphoprotein. In such embodiments, the genetically engineered LVS ΔcapB thereby expresses a MN fusion protein that is presented to immune cells. In illustrative working embodiments of the invention disclosed herein, the at least two antigenic polypeptide epitopes present on a severe acute respiratory syndrome coronavirus 2 polypeptide are encoded by a sequence found in SEQ ID NO: 1 (which is a polynucleotide sequence encoding a fusion protein comprising SARS-CoV-2 membrane (M) glycoprotein coupled in frame via an encoded polypeptide linker to a SARS-CoV-2 nucleocapsid (N) phosphoprotein). In certain embodiments, the antigenic polypeptides can be encoded in a codon optimized polynucleotide sequence.

Embodiments of the invention include concurrent administration of one vaccine embodiment of the invention along with one or more other vaccine embodiments using the same vector. Furthermore, a single vector platform vaccine also has the advantage that different vaccines comprising the same vector but expressing different antigens can be safely and effectively administered at the same time. That is, individual LVS ΔcapB vaccines expressing Burkholderia pseudomallei (Bp) antigens. Francisella tularensis subsp. tularensis (Ft) antigens, Bacillus anthracis (Ba) antigens, Yersinia pestis (Yp) antigens, SARS-CoV-2 antigens, and the antigens of other pathogens, can be administered together.

As discussed in detail below, nine COVID-19 immunogenic vaccine compositions have been constructed and demonstrated to express the relevant SARS-CoV-2 proteins singly and in combination. Embodiments of the invention include an immunogenic composition comprising a recombinant attenuated Francisella tularensis subspecies holarctica Live Vaccine Strain (LVS) having a deletion in a capB gene and which comprises a heterologous promoter that expresses a fusion protein comprising an antigenic polypeptide epitope present in a SARS-CoV-2 virus polypeptide. It is desirable to include large segments of SARS-CoV-2 virus polypeptides in this invention in order to present a large number of immunoreactive epitopes to the mammalian immune system. Optionally the LVS expresses two or more antigenic polypeptide epitopes present in a SARS-CoV-2 virus polypeptide. In this context, illustrative embodiments of the invention include vaccine combinations or combinations of proteins in a single vaccine. Such illustrative combinations include (SARS-CoV-2 proteins bolded):

1. rLVS ΔcapB/SCoV2 (SΔTM)+rLVS ΔcapB/SCoV2 (MN)

2. rLVS ΔcapB/SCoV2 (S1)+rLVS ΔcapB/SCoV2 (MN)

3. rLVS ΔcapB/SCoV2 (S)+rLVS ΔcapB/SCoV2 (MN)

4. rLVS ΔcapB/SCoV2 (S2)+rLVS ΔcapB/SCoV2 (MN)

5. rLVS ΔcapB/SCoV2 (S2E)+rLVS ΔcapB/SCoV2 (MN)

6. rLVS ΔcapB/SCoV2 (S1)+rLVS ΔcapB/S2 (S2)

7. rLVS ΔcapB/SCoV2 (S1)+rLVS ΔcapB/SCoV2 (S2E)

Another embodiment of the invention is a method of generating an immune response in a mammal comprising administering one or more of immunogenic compositions disclosed herein to the mammal so that an immune response is generated to the one or more antigenic polypeptide epitopes present in a SARS-CoV-2 virus polypeptide. In one such embodiment, the method comprises administering an LVS immunogenic composition disclosed herein in a primary vaccination; and administering the same immunogenic composition of LVS immunogenic composition disclosed herein in a subsequent homologous booster vaccination. Typically, the method consists essentially of administering the immunogenic composition of an LVS immunogenic composition disclosed herein in a primary vaccination; and administering the immunogenic composition of LVS immunogenic composition disclosed herein in a subsequent homologous booster vaccination. Optionally, the method comprises administering the immunogenic composition to the mammal less than 4 times.

In another embodiment of the invention, the method comprises administering an LVS composition as disclosed herein in a primary vaccination; and administering a second heterologous immunogenic composition comprising the antigenic polypeptide epitope present in a SARS-CoV-2 virus in a subsequent booster vaccination. Optionally, the second immunogenic composition comprises an attenuated strain of Listeria monocytogenes expressing the antigenic polypeptide epitope. In certain embodiments, the method comprises administering LVS immunogenic composition disclosed herein and a second immunogenic composition to the mammal less than a total of four times. Optionally for example, the method comprises administering a single dose of a first LVS immunogenic composition disclosed herein, and one or more doses of a second immunogenic composition disclosed herein.

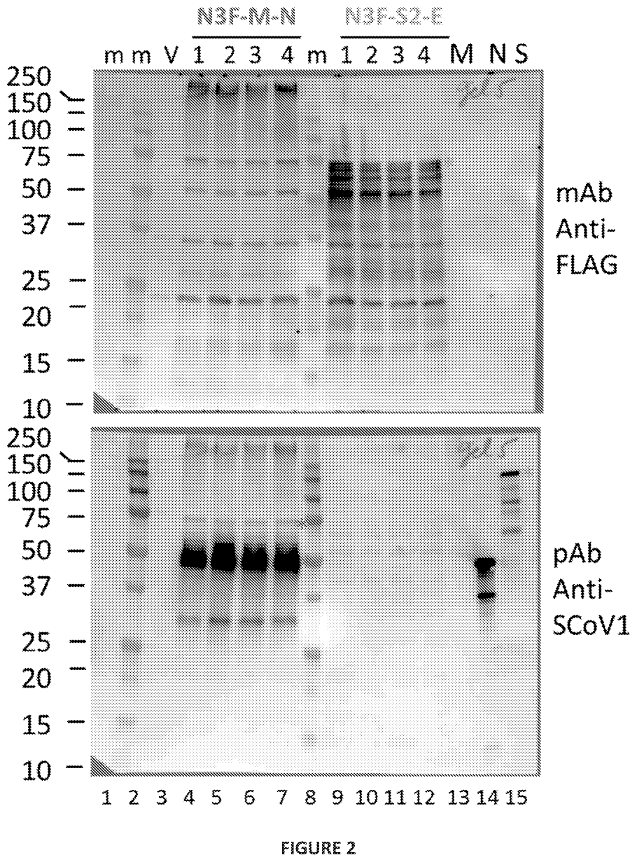

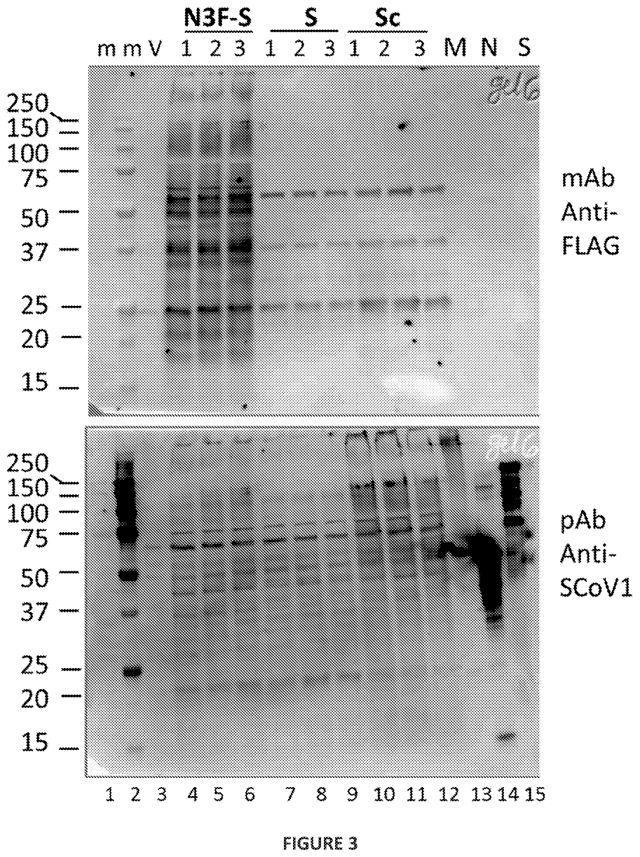

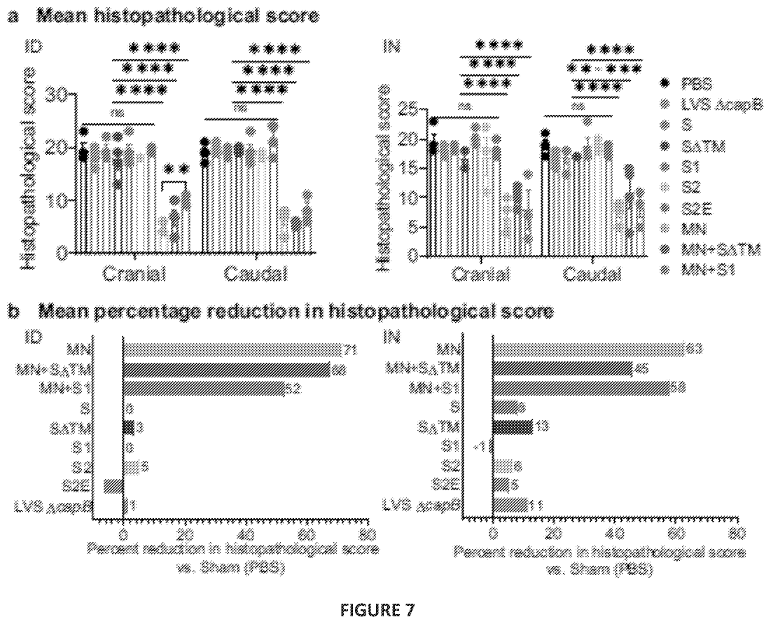

Studies illustrating aspects and properties of the invention are published in Jia et al., NPJ Vaccines. 2021 Mar. 30; 6(1):47. doi: 10.1038/s41541-021-00321-8, the contents of which are incorporated by reference. FIG. 2 in this publication shows that only the MN expressing vaccines protected against severe weight loss, whether administered intradermally (ID) or intranasally (IN), whereas none of the S protein vaccines protected against severe weight loss. FIG. 3 in this publication shows that only the MN expressing vaccines protected against severe lung histopathology, as scored by a pathologist blinded to the identity of the vaccines, whether the vaccines were administered intradermally (ID) or intranasally (IN), whereas none of the S protein vaccines protected against severe lung histopathology. FIG. 5 in this publication shows that only the MN expressing vaccines preserved a high percentage of alveolar air space, whether administered intradermally (ID) or intranasally (IN), whereas none of the S protein vaccines preserved a high percentage of alveolar air space, and that the percent alveolar air space correlated inversely with the histopathological score. FIG. 7 in this publication shows that anti-N antibody is induced only by the MN expressing vaccines, as expected, whether administered intradermally (ID) or intranasally (IN), and that it strongly correlates with protection against lung histopathology. This was unexpected because anti-N antibody is not neutralizing antibody (i.e. it does not neutralize virus infection of mammalian cells) and hence would not be expected to be protective. Without being bound by a specific theory or mechanism of action, it is believed that the anti-N antibody is correlated with induction of T cell responses to the N protein and that it is these T cell responses that are highly protective.

### EXAMPLES

Construction and Characterization of Recombinant LVS ΔcapB Expressing SARS-CoV-2 Antigens

### SARS-CoV-2 Antigen Selection.

The complete genome sequence of SARS-CoV-2 and the polypeptides encoded by this genome are known in the art. See, e.g. “Complete Genome Sequence of a 2019 Novel Coronavirus (SARS-CoV-2) Strain Isolated in Nepal”, Sah et al., Microbiology Resource Announcements March 2020.9 (11) e00169-20; DOI: 10.1128/MRA.00169-20, the contents of which are incorporated by reference; and SARS-CoV-2 sequenced genomes are available at GenBank (e.g. MN988668 and NC_045512, the contents of which are incorporated by reference). See also Zhou P, Yang X L, Wang X G, Hu B. Zhang L, Zhang W, Si H R, Zhu Y, Li B, Huang C L, Chen H D, Chen J, Luo Y. Guo H, Jiang R D, Liu M Q, Chen Y, Shen X R, Wang X, Zheng X S, Zhao K, Chen Q J, Deng F, Liu L L, Yan B. Zhan F X, Wang Y Y, Xiao G F, Shi Z L. A pneumonia outbreak associated with a new coronavirus of probable bat origin. Nature. 2020; 579(7798):270-3. Epub 2020/02/06. doi: 10.1038/s41586-020-2012-7. PubMed PMID: 32015507. See also Wu et al, Nature volume 579, pages 265-269 (2020) and Genebank MT152824 (US), the contents of which are incorporated by reference, for the complete genomic sequence which was used herein for gene optimization.

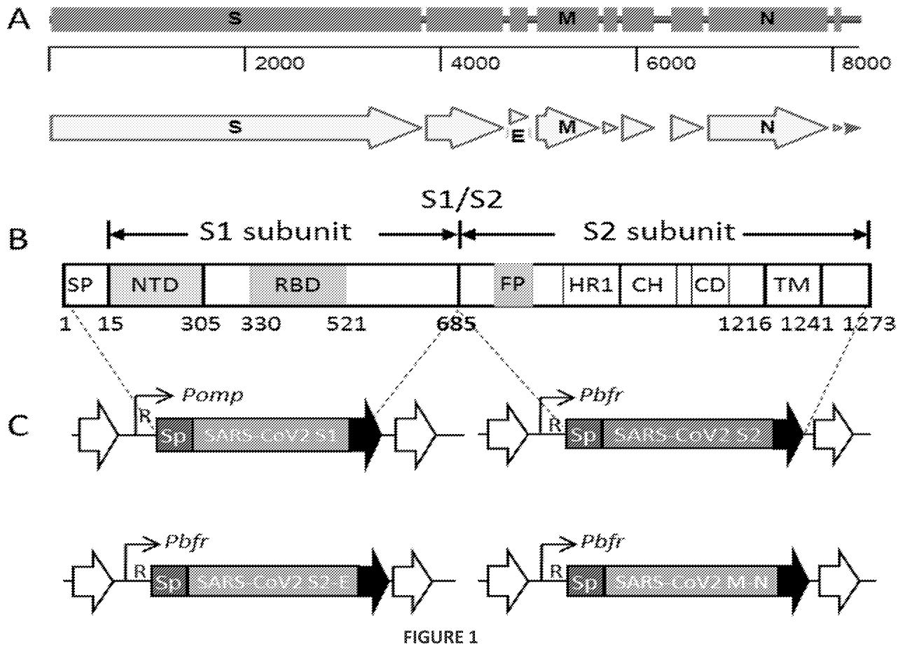

Similar to other coronaviruses, including SARS-CoV and MERS-CoV. SARS-CoV-2 encodes 4 structural proteins: a large surface spike (S) glycoprotein (1273 aa) (1, 3); an envelope (E) protein (75 aa); a membrane (M) glycoprotein (222 aa); and a nucleocapsid (N) phosphoprotein (419 aa) (FIG. 1A). The S protein is synthesized as a single-chain inactive precursor of 1,273 residues with a signal peptide (residue 1-15) and processed by a furin-like host proteinase into the S1 (75 kDa) subunit that binds to host receptor angiotensin converting enzyme II (ACE2) (4), and the S2 (64 kDa) subunit that mediates the fusion of the viral and host cell membranes. The S1 subunit contains host receptor binding domain (RBD) and the S2 subunit contains the fusion peptide (FP), two heptad repeats (HR), and a transmembrane domain (TM) (FIG. 1B). We constructed nine pFNL-derived shuttle plasmids and nine corresponding rLVS ΔcapB-vectored vaccines expressing 1) the S protein with or without an N-terminal tag (S); 2) S protein with a deleted transmembrane domain with an N-terminal tag (SΔTM); 3) the S1 subunit with an N-terminal tag (S1); 4) S2 subunit (S2); 5) S2 protein fused to the E protein with or without an N-terminal tag (S2-E); and 6) the M protein fused to the N protein with or without an N-terminal tag (MN) (FIG. 1C, bottom panels). The expression of the SARS-CoV-2 proteins is driven by a strong Ft promoter (pbfr or pomp) that we have used for vaccines against Ft, Ba. Yp, and Bp. We have tested the efficacy of each vaccine candidate in animals. On the basis of the efficacy results, we shall select the best antigens and construct a final vaccine that expresses the most protective protein antigen(s).

Construction and Verification of rLVS ΔcapB Prime Vaccines Expressing Immunogenic SARS-CoV-2 Antigens.
1A. Construct rLVS ΔcapB Vaccines Expressing SARS-CoV-2 Antigens (rLVS ΔcapB/SCoV2).

We previously have successfully constructed rLVS ΔcapB vaccines expressing shuttle plasmid-encoded Ft, Ba, Yp, and Bp antigens and demonstrated potent protection by the rLVS ΔcapB vaccines against lethal respiratory challenge with the relevant pathogens. We now have used a similar approach to construct vaccines against SARS-CoV-2. For expression of the S protein (protein id QIH55221.1), a gene encoding full-length SARS-CoV-2 S (Genebank MT152824) with two stabilizing proline substitutions at the S2 fusion machinery (K986P and V987P) (1, 5) was codon-optimized for expression in LVS ΔcapB and synthesized by Atum.com. Similarly, genes encoding SARS-CoV-2 E, M, N proteins were also codon-optimized and synthesized by Atum.com. The synthesized genes encoding the full-length S protein (145 kDa), the fusion proteins of S2-E (72 kDa), and the fusion protein of MN (71 kDa) linked by flexible linker (GGSG) were cloned separately into a pFNL-derived expression shuttle plasmid downstream of the pbfr promoter by the Electra Cloning System (ATUM) and traditional molecular cloning methods (6). Subsequently we performed a deletional mutagenesis of the codon-optimized gene for full-length S protein to generate pFNL-derived expression shuttle plasmids for SΔTM. S1 and S2 subunits. We shall also construct a pFNL-derived shuttle plasmid carrying expression cassettes for both S1 and S2 subunits driven by the Francisella omp and bfr promoter, respectively, as indicated in FIG. 1C, top panels. Each antigen expression cassette in the shuttle plasmid is composed of the following elements: Ft bfr or Fn omp promoter followed by a ribosomal entry site (Shine-Dalgarno sequence), 6 nucleotide spacer, and the nucleotide sequences encoding the SARS-CoV-2 proteins. The expression shuttle plasmid, carrying a kanamycin-resistance gene, was verified by restriction analysis and/or nucleotide sequencing and electroporated into LVS ΔcapB electro-competent cells; recombinant clones (rLVS ΔcapB expressing S, SΔTM, S1, S2, S2-E, and MN) were selected on chocolate agar plates supplemented with kanamycin; kanamycin-resistant clones were verified for expression of the targeted proteins and by restriction analysis of the shuttle plasmids isolated from the vaccine strain.

As expected, the fusion protein of MN with or without N-terminal tags were abundantly expressed by the LVS ΔcapB vector and recognized by the guinea pig polyclonal antibody to SARS CoV (NR-10361, BEI Resources). Surprisingly, the full-length Spike protein (145 kDa) was also abundantly expressed by the LVS ΔcapB vector and recognized by the guinea pig polyclonal antibody to SARS CoV (NR-10361, BEI Resources). This is the largest protein we have successfully expressed from the LVS ΔcapB vector. The SΔTM, S1, and S2 were also expressed by the LVS ΔcapB vector as demonstrated by Western blotting analysis by using the monoclonal antibody to the N-terminal tag (FLAG) and by using the polyclonal antibody to SARS CoV.

1B. Characterize rLVS ΔcapB Vaccines In Vitro, Including Protein Expression and Growth Kinetics in Broth and in Macrophages, and Genetic Stability of the Integrated Antigen Expression Cassette.
1B1. Protein Expression by rLVS ΔcapB/SCoV2 Vaccine Grown on Agar Plates.

Heterologous protein expression by rLVS ΔcapB/SCoV2 vaccines on Chocolate agar plates were analyzed by Western blotting using polyclonal antibody to SARS-CoV or monoclonal antibodies to the N-terminal tags of the SCoV2 protein, as described by us previously (7-9).

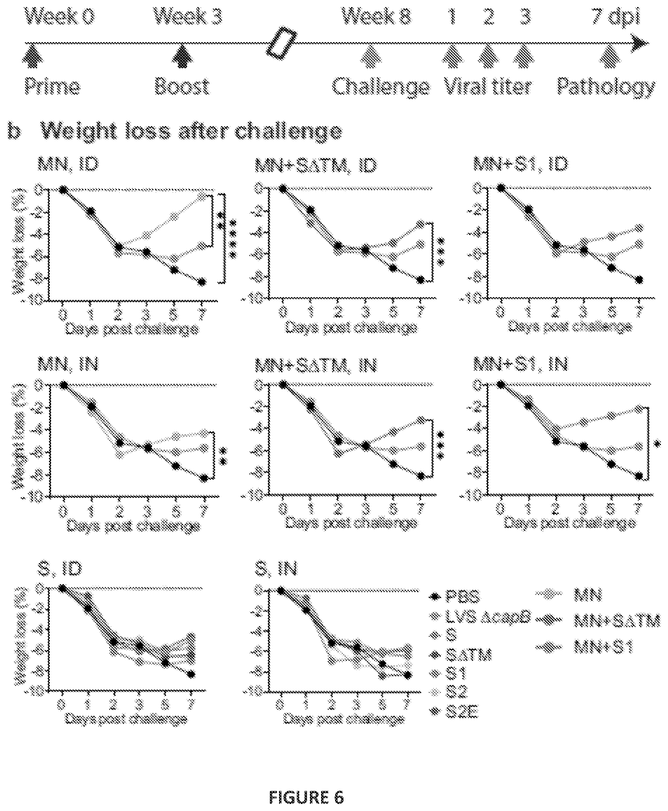

In studies of embodiments of the invention disclosed herein, a major unexpected finding was that only the vaccines expressing the Membrane (M) and Nucleocapsid (N) proteins (e.g. the MN fusion protein of SEQ ID NO: 1) were protective (either when administered alone or with vaccines expressing other proteins), whereas all of the vaccines expressing only the S protein (or a part of the S protein i.e. SΔTM, S1, or S2) or the S2 protein fused to the Envelope (E) protein (S2E) were not protective. It was also unexpected that the MN fusion protein expressing vaccines worked just as well when administered by the intranasal route as by the intradermal route. Specifically, we used the LVS ΔcapB vector platform to construct six COVID-19 vaccines expressing one or more of all four structural proteins of SARS-CoV-2 and tested the vaccines for efficacy, administered intradermally (ID) or intranasally (IN), against a high dose SARS-CoV-2 respiratory challenge in hamsters. These studies showed that the LVS ΔcapB vaccine expressing COVID-19 MN proteins, but not the vaccines expressing the S protein or its subunits in various configurations, is highly protective against severe COVID-19-like disease including weight loss and lung pathology, and also that protection is highly correlated with serum anti-N antibody levels. See FIGS. 6 and 7.

### CONCLUSION

This concludes the description of embodiments of the present invention. The foregoing description of one or more embodiments of the invention has been presented for the purposes of illustration and description. It is not intended to be exhaustive or to limit the invention to the precise form disclosed. Many modifications and variations are possible in light of the above teaching.

### REFERENCES

1. Wrapp D, Wang N, Corbett K S, Goldsmith J A, Hsieh C L, Abiona O, Graham B S, McLellan J S. Cryo-EM structure of the 2019-nCoV spike in the prefusion conformation. Science. 2020; 367(6483):1260-3. Epub 2020/02/23. doi: 10.1126/science.abb2507. PubMed PMID: 32075877.
2. Limmathurotsakul D, Golding N, Dance D A, Messina J P, Pigott D M, Moyes C L, Rolim D B, Bertherat E, Day N P, Peacock S J, Hay S I. Predicted global distribution of Burkholderia pseudomallei and burden of melioidosis. Nature microbiology. 2016; 1:15008. doi: 10.1038/nmicrobiol.2015.8. PubMed PMID: 27571754.
3. Walls A C, Park Y J, Tortorici M A, Wall A. McGuire A T, Veesler D. Structure, Function, and Antigenicity of the SARS-CoV-2 Spike Glycoprotein. Cell. 2020. Epub 2020/03/11. doi: 10.1016/j.cell.2020.02.058. PubMed PMID: 32155444.
4. Zhou P, Yang X L, Wang X G, Hu B, Zhang L, Zhang W, Si H R, Zhu Y, Li B, Huang C L, Chen H D, Chen J, Luo Y, Guo H, Jiang R D, Liu M Q, Chen Y, Shen X R, Wang X, Zheng X S, Zhao K, Chen Q J, Deng F, Liu L L, Yan B, Zhan F X, Wang Y Y, Xiao G F, Shi Z L. A pneumonia outbreak associated with a new coronavirus of probable bat origin. Nature. 2020; 579(7798):270-3. Epub 2020/02/06. doi: 10.1038/s41586-020-2012-7. PubMed PMID: 32015507.
5. Pallesen J, Wang N, Corbett K S, Wrapp D, Kirchdoerfer R N, Turner H L, Cottrell C A, Becker M M, Wang L, Shi W, Kong W P, Andres E L, Kettenbach A N, Denison M R, Chappell J D, Graham B S, Ward A B, McLellan J S. Immunogenicity and structures of a rationally designed prefusion MERS-CoV spike antigen. Proc Natl Acad Sci USA. 2017:114(35):E7348-E57. Epub 2017/08/16. doi: 10.1073/pnas.1707304114. PubMed PMID: 28807998; PMCID: PMC5584442.
6. Jia Q, Bowen R, Dillon B J, Maslesa-Galic S, Chang B T, Kaidi A C, Horwitz M A. Single vector platform vaccine protects against lethal respiratory challenge with Tier 1 select agents of anthrax, plague, and tularemia. Scientific reports. 2018; 8(1):7009. Epub 2018/05/05. doi: 10.1038/s41598-018-24581-y. PubMed PMID: 29725025; PMCID: PMC5934503.
7. Lee B Y, Horwitz M A, Clemens D L. Identification, recombinant expression, immunolocalization in macrophages, and T-cell responsiveness of the major extracellular proteins of Francisella tularensis. Infect Immun. 2006; 74(7):4002-13. doi: 10.1128/IAI.00257-06. PubMed PMID: 16790773: PMCID: 1489726.
8. Jia Q, Lee B Y, Clemens D L, Bowen R A, Horwitz M A. Recombinant attenuated Listeria monocytogenes vaccine expressing Francisella tularensis Ig1C induces protection in mice against aerosolized Type A F. tularensis. Vaccine. 2009; 27(8):1216-29. Epub 2009/01/08. doi: 10.1016/j.vaccine.2008.12.014. PubMed PMID: 19126421: PMCID: 2654553.
9. Jia Q, Bowen R, Lee B Y, Dillon B J, Maslesa-Galic S, Horwitz M A. Francisella tularensis Live Vaccine Strain deficient in capB and overexpressing the fusion protein of Ig1A, Ig1B, and Ig1C from the bfr promoter induces improved protection against F. tularensis respiratory challenge. Vaccine. 2016; 34(41):4969-78. doi: 10.1016/j.vaccine.2016.08.041. PubMed PMID: 27577555; PMCID: 5028307.
10. Jia Q, Lee B Y, Bowen R, Dillon B J, Som S M, Horwitz M A. A Francisella tularensis Live Vaccine Strain (LVS) mutant with a deletion in capB, encoding a putative capsular biosynthesis protein, is significantly more attenuated than LVS yet induces potent protective immunity in mice against F. tularensis challenge. Infect Immun. 2010; 78(10):4341-55. Epub 2010/07/21. doi: 10.1128/IAI.00 192-10. PubMed PMID: 20643859; PMCID: 2950357.

### SEVERE ACUTE RESPIRATORY SYNDROME CORONAVIRUS 2 (SARS-COV-2) POLYNUCLEOTIDE SEQUENCES USEFUL IN EMBODIMENTS OF THE INVENTION

1. SCoV2 MN (1938 bp)
ATGGCTGATAGCAATGGAACGATTACAGTAGAAGAGTTAAAAAAACTTCTAGAGCAATGGAATCT
TGTAATTGGCTTTCTATTTCTAACATGGATATGTCTATTACAGTTTGCTTATGCCAATAGAAATA
GATTTCTTTATATAATAAAACTTATCTTTCTATGGCTATTATGGCCTGTTACATTAGCTTGTTTC
GTTCTAGCTGCTGTTTATAGAATAAATTGGATAACCGGTGGAATTGCAATTGCTATGGCCTGGTT
AGTCGGACTTATGTGGCTTTCATATTTTATTGCCTCATTTCGATTATTCGCTAGAACACGCTCGA
TGTGGAGCTTTAATCCAGAAACTAATATATTATTAAATGTGCCATTACATGGTACAATTTTGACT
AGACCTCTTTTAGAAAGCGAATTAGTTATAGGTGCAGTTATCCTACGTGGACATTTAAGAATTGC
TGGCCACCATCTTGGTAGATGTGATATCAAAGATTTACCAAAAGAAATAACTGTAGCAACATCTA
GAACATTATCATATTATAAATTGGGTGCTTCACAGAGAGTGGCGGGTGATTCAGGTTTTGCAGCT
TATTCTAGGTATAGGATTGGTAACTATAAATTGAATACGGATCACAGTTCCTCAAGTGATAATAT
TGCACTTCTTGTACAGGGTGGTAGCGGTATGTCAGATAACGGTCCTCAAAATCAAAGAAATGCTC
CTAGAATAACTTTTGGTGGCCCAAGTGATAGTACTGGTAGTAATCAGAACGGTGAGAGAAGTGGA
GCAAGATCTAAGCAACGCAGACCGCAAGGGCTACCTAATAATACTGCGTCATGGTTTACTGCTTT
AACACAACATGGTAAAGAAGATTTAAAGTTTCCTCGCGGTCAGGGTGTTCCAATTAATACTAATA
GTTCGCCAGATGATCAAATTGGTTATTATCGTCGTGCTACTAGACGAATTCGTGGTGGCGACGGA
AAAATGAAAGATCTATCTCCACGTTGGTACTTTTACTATTTAGGTACCGGTCCAGAGGCTGGTTT
ACCTTATGGTGCTAACAAAGACGGGATAATATGGGTCGCTACCGAGGGTGCACTTAATACGCCAA
AAGATCATATCGGAACTCGTAACCCAGCAAATAACGCTGCTATTGTTTTACAATTACCTCAAGGT
ACTACACTGCCTAAAGGTTTCTATGCAGAGGGCTCTAGGGGTGGAAGCCAAGCATCAAGTCGTTC
AAGTTCTCGTAGCAGAAACTCTTCTAGAAATAGTACTCCTGGCTCATCACGTGGAACAAGTCCAG
CGAGAATGGCTGGTAATGGCGGGGATGCAGCATTAGCATTGTTACTTTTAGATAGATTAAATCAG
CTTGAATCTAAAATGTCTGGCAAAGGGCAACAACAACAGGGTCAAACAGTAACTAAGAAATCAGC
AGCAGAGGCTTCGAAGAAACCTAGACAGAAGAGAACTGCTACAAAAGCGTACAATGTTACGCAAG
CATTTGGCCGCAGAGGACCAGAACAGACTCAAGGGAATTTTGGTGATCAAGAATTAATTCGTCAA
GGTACAGATTATAAACATTGGCCCCAGATAGCTCAATTTGCTCCATCTGCATCTGCATTCTTTGG
AATGTCAAGAATTGGTATGGAAGTTACTCCTAGTGGAACTTGGCTAACTTATACTGGTGCTATAA
AGCTCGATGATAAAGATCCTAATTTTAAAGATCAAGTAATTTTGTTAAACAAGCATATAGATGCA
TATAAAACATTTCCTCCTACTGAACCAAAAAAAGATAAAAAGAAAAAAGCTGACGAAACACAAGC
TCTACCGCAAAGGCAAAAGAAACAACAGACAGTAACATTATTGCCAGCTGCTGATTTAGATGATT
TTTCAAAACAACTTCAACAATCTATGAGTAGCGCAGATAGTACTCAAGCATAA (SEQ ID NO:
1)
2. SCoV2 S2-E (1998 bp)
ATGTCTGTAGCGAGTCAATCAATAATAGCATATACAATGTCATTAGGCGCAGAAAATAGTGTGGC
TTATTCTAATAATTCTATCGCAATCCCTACCAATTTCACTATAAGTGTTAGAACCGAAATCTTAG
CAGTTAGTATGACAAAGACAAGTGTTGATTGTACTATGTATATATGTGGCGATTCTACTGAGTGT
TCTAATCTCTTATTACAATATGGTTCGTTTTGTACTCAGTTAAATCGAGCTCTTACAGGTATAGC
TGTCGAGCAAGATAAGAATACCCAGGAAGTCTTTGCACAGGTTAAACAAATTTATAAAACTCCAC
CAATCAAAGATTTTGGTGGGTTTAACTTTTCTCAAATACTACCTGATCCATCTAAACCCTCTAAA
CGTAGTTTTATTGAAGATTTACTTTTTAATAAAGTAACTCTAGCTGATGCTGGTTTCATTAAACA
ATACGGCGATTGTTTGGGTGATATAGCGGCACGTGATTTAATATGCGCACAGAAATTCAACGGTC
TGACAGTCCTACCTCCATTATTGACAGATGAAATGATTGCTCAATATACATCAGCATTGCTTGCT
GGCACTATCACGAGTGGATGGACTTTTGGTGCTGGCGCTGCTTTACAAATTCCATTTGCCATGCA
AATGGCTTATAGATTTAATGGTATTGGTGTTACACAAAATGTTTTATATGAGAATCAAAAGTTAA
TAGCTAACCAATTTAACTCTGCAATTGGCAAGATTCAGGATTCATTATCTAGTACAGCGAGTGCT
TTAGGTAAACTACAAGATGTAGTGAATCAGAATGCTCAAGCACTCAATACTTTGGTTAAACAATT
AAGTTCAAATTTTGGTGCAATTTCAAGTGTACTAAATGATATTCTAAGTCGCTTAGATAAAGTTG
AGGCTGAAGTACAAATCGATAGACTAATTACAGGTAGATTACAGTCATTACAAACTTATGTTACT
CAACAGTTAATTAGAGCTGCAGAAATAAGAGCATCTGCAAATTTGGCAGCCACTAAGATGAGTGA
GTGTGTCCTTGGACAATCAAAACGTGTAGATTTTTGCGGAAAGGGATATCACTTAATGTCATTTC
CGCAATCTGCACCTCATGGTGTCGTGTTTCTTCATGTTACTTACGTTCCGGCTCAAGAGAAAAAC
TTCACTACGGCTCCAGCGATTTGTCATGATGGTAAAGCTCATTTTCCTCGTGAGGGTGTATTTGT
ATCAAATGGAACACATTGGTTTGTTACTCAAAGAAATTTTTATGAGCCACAAATAATAACTACAG
ATAATACTTTTGTTAGCGGTAACTGTGACGTAGTTATAGGAATCGTAAACAACACAGTGTATGAT
CCATTACAACCAGAGTTAGATTCTTTTAAAGAAGAACTTGATAAGTATTTCAAAAATCATACTAG
CCCTGATGTTGACCTTGGTGACATATCAGGCATAAATGCATCAGTTGTTAATATTCAAAAAGAAA
TAGATAGGCTTAATGAAGTTGCTAAAAATCTTAATGAATCTTTAATAGATCTACAAGAACTTGGA
AAATACGAACAATATATAAAATGGCCTTGGTATATATGGTTAGGGTTTATTGCTGGTCTTATTGC
TATTGTAATGGTAACTATTATGCTATGTTGTATGACATCATGCTGTAGCTGTCTAAAGGGTTGTT
GTAGTTGTGGTTCATGTTGCAAATTTGATGAAGATGATAGTGAGCCAGTTCTTAAAGGTGTAAAA
TTGGGGGGATCTGGAATGTACAGCTTTGTGTCAGAAGAAACCGGTACACTAATTGTTAATAGCGT
TTTACTTTTTCTGGCTTTTGTTGTGTTTCTTCTAGTAACATTGGCCATCTTGACTGCACTAAGAC
TTTGTGCTTATTGCTGTAATATTGTTAATGTTTCATTAGTAAAACCTAGCTTTTATGTTTATTCG
AGAGTCAAAAACCTAAATTCCAGTAGAGTACCTGATTTATTAGTATAA (SEQ ID NO: 2)
3. SCov2 S2P (3822 bp)
ATGTTTGTGTTTTTAGTTCTTTTACCGTTAGTTTCAAGTCAATGTGTGAACTTAACTACACGCAC
ACAACTTCCTCCAGCATATACAAATAGTTTTACTAGAGGTGTATATTATCCTGATAAAGTATTCC
GTAGTTCTGTTCTACATTCTACACAAGATTTGTTTTTACCGTTTTTCAGTAATGTCACTTGGTTC
CATGCTATTCATGTTTCTGGGACAAACGGTACAAAAAGATTTGATAACCCTGTTTTACCATTTAA
TGATGGTGTATATTTTGCTTCAACTGAGAAAAGCAATATAATTAGAGGTTGGATTTTCGGAACTA
CCCTGGATAGCAAGACGCAAAGTTTATTGATCGTAAACAATGCTACAAACGTCGTAATTAAAGTA
TGTGAATTTCAATTTTGTAATGACCCTTTTTTAGGAGTCTATTATCATAAAAATAATAAATCTTG
GATGGAGTCTGAATTTAGAGTTTATTCTAGCGCTAATAACTGTACATTTGAATATGTTTCACAAC
CTTTTTTAATGGATCTAGAAGGTAAACAGGGTAATTTTAAAAATCTTCGTGAGTTTGTTTTTAAG
AACATAGATGGATATTTCAAAATATATTCAAAACATACTCCTATTAATCTAGTTAGAGATCTTCC
ACAAGGCTTTTCTGCTCTAGAACCATTAGTTGATTTACCAATAGGTATAAATATAACTCGTTTCC
AAACTTTACTAGCCCTTCACCGTTCGTACTTAACGCCTGGGGATTCTTCTAGTGGTTGGACTGCT
GGCGCTGCAGCATATTATGTTGGATATCTACAACCTAGAACATTTTTATTGAAATACAACGAAAA
CGGAACTATAACTGACGCTGTTGATTGTGCACTTGATCCATTAAGTGAGACTAAATGTACTCTAA
AAAGTTTTACTGTTGAAAAGGGAATTTATCAAACATCAAATTTTCGCGTTCAACCAACGGAAAGT
ATTGTACGTTTTCCGAACATAACCAATTTATGTCCTTTCGGTGAGGTATTTAACGCAACTCGTTT
TGCGAGCGTATATGCTTGGAATAGAAAAAGAATTAGCAATTGTGTTGCTGATTATTCGGTCTTAT
ACAATAGTGCTTCGTTTAGCACTTTTAAATGTTACGGAGTAAGTCCAACAAAGTTAAATGATCTA
TGTTTCACTAATGTGTATGCTGATTCTTTTGTTATTAGAGGTGATGAAGTTCGACAAATTGCTCC
AGGTCAAACTGGCAAAATTGCGGACTATAATTATAAGCTACCTGATGATTTTACTGGCTGTGTGA
TTGCATGGAATAGTAATAATCTAGATTCGAAAGTCGGTGGGAATTATAATTATCTTTATAGACTA
TTTAGAAAATCTAATTTGAAACCATTTGAGAGAGATATATCAACAGAAATTTACCAGGCTGGCAG
CACACCTTGCAACGGCGTAGAAGGTTTTAATTGTTATTTTCCACTACAAAGTTATGGTTTTCAAC
CAACTAATGGCGTCGGGTATCAACCATATAGAGTTGTCGTACTTTCCTTTGAATTACTTCATGCA
CCAGCTACCGTTTGTGGGCCAAAGAAATCAACTAATCTTGTAAAGAATAAATGCGTCAATTTTAA
TTTTAATGGCCTTACAGGCACTGGAGTTTTAACAGAATCCAATAAAAAATTTTTACCTTTTCAGC
AATTTGGTAGAGATATAGCTGATACTACTGATGCTGTAAGAGATCCTCAAACTCTAGAGATTTTA
GATATTACCCCGTGTTCATTTGGAGGCGTAAGCGTTATAACTCCAGGCACGAACACATCAAATCA
AGTTGCTGTACTATATCAAGATGTTAATTGCACAGAAGTGCCTGTTGCCATTCATGCAGATCAAC
TTACTCCTACATGGCGTGTATATTCTACCGGATCAAATGTATTTCAGACTAGAGCTGGTTGTTTA
ATAGGCGCAGAACATGTAAATAATAGTTATGAGTGTGATATACCAATTGGTGCAGGAATATGTGC
ATCATATCAGACACAGACAAATAGTCCTCGTCGCGCAAGATCAGTAGCATCACAATCGATTATAG
CTTATACAATGTCTTTAGGTGCGGAAAATAGTGTGGCTTATTCTAATAATTCTATCGCAATCCCT
ACCAATTTCACTATAAGTGTTACAACCGAAATCTTACCAGTTAGTATGACAAAGACAAGTGTTGA
TTGTACTATGTATATATGTGGCGATTCTACTGAGTGTTCTAATCTCTTATTACAATATGGTTCGT
TTTGTACTCAGTTAAATCGAGCTCTTACAGGTATAGCTGTCGAGCAAGATAAGAATACCCAGGAA
GTCTTTGCACAGGTTAAACAAATTTATAAAACTCCACCAATCAAAGATTTTGGTGGGTTTAACTT
TTCTCAAATACTACCTGATCCATCTAAACCCTCTAAACGTAGTTTTATTGAAGATTTACTTTTTA
ATAAAGTAACTCTAGCTGATGCTGGTTTCATTAAACAATACGGCGATTGTTTGGGTGATATAGCG
GCACGTGATTTAATATGCGCACAGAAATTCAACGGTCTGACAGTCCTACCTCCATTATTGACAGA
TGAAATGATTGCTCAATATACATCAGCATTGCTTGCTGGCACTATCACGAGTGGATGGACTTTTG
GTGCTGGCGCTGCTTTACAAATTCCATTTGCCATGCAAATGGCTTATAGATTTAATGGTATTGGT
GTTACACAAAATGTTTTATATGAGAATCAAAAGTTAATAGCTAACCAATTTAACTCTGCAATTGG
CAAGATTCAGGATTCATTATCTAGTACAGCGAGTGCTTTAGGTAAACTACAAGATGTAGTGAATC
AGAATGCTCAAGCACTCAATACTTTGGTTAAACAATTAAGTTCAAATTTTGGTGCAATTTCAAGT
GTACTAAATGATATTCTAAGTCGCTTAGATCCTCCAGAGGCTGAAGTACAAATCGATAGACTAAT
TACAGGTAGATTACAGTCATTACAAACTTATGTTACTCAACAGTTAATTAGAGCTGCAGAAATAA
GAGCATCTGCAAATTTGGCAGCCACTAAGATGAGTGAGTGTGTCCTTGGACAATCAAAACGTGTA
GATTTTTGCGGAAAGGGATATCACTTAATGTCATTTCCGCAATCTGCACCTCATGGTGTCGTGTT
TCTTCATGTTACTTACGTTCCGGCTCAAGAGAAAAACTTCACTACGGCTCCAGCGATTTGTCATG
ATGGTAAAGCTCATTTTCCTCGTGAGGGTGTATTTGTATCAAATGGAACACATTGGTTTGTTACT
CAAAGAAATTTTTATGAGCCACAAATAATAACTACAGATAATACTTTTGTTAGCGGTAACTGTGA
CGTAGTTATAGGAATCGTAAACAACACAGTGTATGATCCATTACAACCAGAGTTAGATTCTTTTA
AAGAAGAACTTGATAAGTATTTCAAAAATCATACTAGCCCTGATGTTGACCTTGGTGACATATCA
GGCATAAATGCATCAGTTGTTAATATTCAAAAAGAAATAGATAGGCTTAATGAAGTTGCTAAAAA
TCTTAATGAATCTTTAATAGATCTACAAGAACTTGGAAAATACGAACAATATATAAAATGGCCTT
GGTATATATGGTTAGGGTTTATTGCTGGTCTTATTGCTATTGTAATGGTAACTATTATGCTATGT
TGTATGACATCATGCTGTAGCTGTCTAAAGGGTTGTTGTAGTTGTGGTTCATGTTGCAAATTTGA
TGAAGATGATAGTGAGCCAGTTCTTAAAGGTGTAAAATTGCATTACACATGA (SEQ ID NO:
3)
4. pFNLdAp-b£r-N3F8H-SCoV2_(MN) (8340 bp)
ggtacctggttactattgccatcatcacaatattaaaattaattttcttcatttatttttcttaa
atattattattaaaaatagtaaatttaacttatctaaaaatagcataatatcatttttattaaaa
tatctaggttgaattcttagatattttgatatataattagatactaaattgataacttataaaga
attaaattttcttttgtatgctaacttgattgctaatatgaattatactagttagtatgttgatt
ataataattaaaattttaaataataaaaataacaataaaaaatacataataaattataaaaatca
cgatggtgattacaaagaccatgatatagattataaggatgacgatgataagcatcatcatcacc
accatcatcatggaggtggttcaATGGCTGATAGCAATGGAACGATTACAGTAGAAGAGTTAAAA
AAACTTCTAGAGCAATGGAATCTTGTAATTGGCTTTCTATTTCTAACATGGATATGTCTATTACA
GTTTGCTTATGCCAATAGAAATAGATTTCTTTATATAATAAAACTTATCTTTCTATGGCTATTAT
GGCCTGTTACATTAGCTTGTTTCGTTCTAGCTGCTGTTTATAGAATAAATTGGATAACCGGTGGA
ATTGCAATTGCTATGGCCTGCTTAGTCGGACTTATGTGGCTTTCATATTTTATTGCCTCATTTCG
ATTATTCGCTAGAACACGCTCGATGTGGAGCTTTAATCCAGAAACTAATATATTATTAAATGTGC
CATTACATGGTACAATTTTGACTAGACCTCTTTTAGAAAGCGAATTAGTTATAGGTGCAGTTATC
CTACGTGGACATTTAAGAATTGCTGGCCACCATCTTGGTAGATGTGATATCAAAGATTTACCAAA
AGAAATAACTGTAGCAACATCTAGAACATTATCATATTATAAATTGGGTGCTTCACAGAGAGTGG
CGGGTGATTCAGGTTTTGCAGCTTATTCTAGGTATAGGATTGGTAACTATAAATTGAATACGGAT
CACAGTTCCTCAAGTGATAATATTGCACTTCTTGTACAGGGTGGTAGCGGTATGTCAGATAACGG
TCCTCAAAATCAAAGAAATGCTCCTAGAATAACTTTTGGTGGCCCAAGTGATAGTACTGGTAGTA
ATCAGAACGGTGAGAGAAGTGGAGCAAGATCTAAGCAACGCAGACCGCAAGGGCTACCTAATAAT
ACTGCGTCATGGTTTACTGCTTTAACACAACATGGTAAAGAAGATTTAAAGTTTCCTCGCGGTCA
GGGTGTTCCAATTAATACTAATAGTTCGCCAGATGATCAAATTGGTTATTATCGTCGTGCTACTA
GACGAATTCGTGGTGGCGACGGAAAAATGAAAGATCTATCTCCACGTTGGTACTTTTACTATTTA
GGTACCGGTCCAGAGGCTGGTTTACCTTATGGTGCTAACAAAGACGGGATAATATGGGTCGCTAC
CGAGGGTGCACTTAATACGCCAAAAGATCATATCGGAACTCGTAACCCAGCAAATAACGCTGCTA
TTGTTTTACAATTACCTCAAGGTACTACACTGCCTAAAGGTTTCTATGCAGAGGGCTCTAGGGGT
GGAAGCCAAGCATCAAGTCGTTCAAGTTCTCGTAGCAGAAACTCTTCTAGAAATAGTACTCCTGG
CTCATCACGTGGAACAAGTCCAGCGAGAATGGCTGGTAATGGCGGGGATGCAGCATTAGCATTGT
TACTTTTAGATAGATTAAATCAGCTTGAATCTAAAATGTCTGGCAAAGGGCAACAACAACAGGGT
CAAACAGTAACTAAGAAATCAGCAGCAGAGGCTTCGAAGAAACCTAGACAGAAGAGAACTGCTAC
AAAAGCGTACAATGTTACGCAAGCATTTGGCCGCAGAGGACCAGAACAGACTCAAGGGAATTTTG
GTGATCAAGAATTAATTCGTCAAGGTACAGATTATAAACATTGGCCCCAGATAGCTCAATTTGCT
CCATCTGCATCTGCATTCTTTGGAATGTCAAGAATTGGTATGGAAGTTACTCCTAGTGGAACTTG
GCTAACTTATACTGGTGCTATAAAGCTCGATGATAAAGATCCTAATTTTAAAGATCAAGTAATTT
TGTTAAACAAGCATATAGATGCATATAAAACATTTCCTCCTACTGAACCAAAAAAAGATAAAAAG
AAAAAAGCTGACGAAACACAAGGTGTACGGCAAAGGGAAAAGAAACAACAGACAGTAACATTATT
GCCAGCTGCTGATTTAGATGATTTTTCAAAACAACTTCAACAATCTATGAGTAGCGCAGATAGTA
CTCAAGCATAAggttaaggatccactagctcgtttcaaattaccgatgatatoggaccgttccaa
cttaccgaccagttcggcaggtatgtatttgcgtgcattcctatccaaaaaaacatcaagccaaa
agcttgaaaaaacttacaacacagctcaacagagctagattgtaaaaccctgctttgttaagcag
aacgcaaaaattgaatgacttatagtcatatcgcttcgaccctogtagattagtagccttgagct
attaactggttgaaacacttaccaaataaagattaaaagcgataaaaatgaaagataaagcagct
aaaaacagagattttagaaagactattttatcagtgttacaacgcaataaagatggctcttttgc
tacgcaagcaaatagaaagtctattctgttgcaggcaactaaagaccttaaaaaggtagggttta
gcaaggttacagccgaaaacttoggtaataagcattgctatgcacttagagaccattggagagcc
caaggattagctacagcaacgataaaaaatcgtttagcttgtctaaggtggttaggcgagaaaat
gggcaaagaactacccgataatcgaaaattagagattgagaacaggaagtatagcgataattcaa
tcaataaagcccaagaaatcgattttaaggcgatttctgccttaactgataggcaagccctagca
atacaattacagcgcgaatttgggcttcgtagagaagaaagtttgaagtttcagcctagttatgc
aatcaaagagcataaaatcgagcttaaaagctcttggacaaagggtggaagaccacgagaaatcc
caattttgaatgaaaaacagagagaattgttagaaaaagtaaaagaggtagcaggtaaaggctct
ctaattgagagcgaaaagtcttataagcaagcaatggaacatttcacgactogctgtcaaagagc
agggattaagaatgttcatggctttagacatgcgtatgctcaagatagatataggcaattaacag
ggcgtgagtgtcctaaaaatggtggattaacatctaagcagctaacacctgagcaaaagcaacaa
gactatgaagctagaatgactattagtgaagagttaggtcatggtagagaagatgtaacagtcaa
ctacttaggcagataaaaagcaatatagctatagaagaaaagaaagctattttacatagtagatc
gactcttcttagggattttatattttttgataaatcatctattttgctagttaaatcatcaaatt
tatcatcttgttgtttgactaaatctaagaatctattctcttttttaaaatcgttcatgcaaacc
gcctatagctttcttctttttctgaaattatttgtcttcacaccataattaaattcccattttta
taagtaaagtcttttaaaagcttgtcagtctcttctctagaaatgtaccaaattttacctatttt
aggatacttttcatgaagTtcttctatttttccccagtcctttaatagtctacctttagagtctc
gtaaatagttatctttgtgacaggggcctcttttatcttttttaatgtaactatatgttattcca
acgtcactattactattatccaaatcttttttagcatgccagtaagaactttcataacttaactc
tatctttcgacctctttgatatacaacaataaagctatagccagtagtaacaacctgttttactt
ttgttaaatctattaacttcttatttatttttttatgtttttttgaaaatttaaatatttctata
ttcattcctacacttcctcaaatccaaatggtagcttatgattctcttctggtttcttttctaat
ttttttatatttgcaataaaaactctttttctatctttgatttttttattgtcccaattcctcca
agtatcatcacaaaccctttcaatatcatgtaaatgatgatgtctaaatattgatctgacataat
acagatctaggtctagttcatcacttaacacaacttctctaagtctttcagatgcttcgattggt
atgtaatcctctttatttttagtatctaaaagcttttgcttaaattcttcttctgtctctgctac
cttactaactgtaaacttgatatttgtaatcttacgaccatgttttctgtgatgatccttgtcat
cataggttacaaaaatatccgataattgattaatctcttctagtgctggtaataggaacttattt
ttaaaatttgaatatctgttgctgtaacttttaggtaaatcaaaatcattaatcatatcatcgac
atacaatacgcaatcaactatattagcataccctgcttgttcgcctaatttgcttttgagaagta
agtataatctgcttgaatacttacttttaaatgaaaatagtaactgtctttctgctttagtaaag
tactcttgtagttgtatcatgtgtggcattaatgaccaatgaaactogcaaattaaagcactgct
tttagggtctgcttcaatatatgcaaaccagttagctatcttcgtttgttctttattcagccata
ctggcttagacattattgagtgcattaattgcttcaatctcactctgttatgcttaacccctgta
gctttttcaagatcagataggcttatcttatacctgtgaaactctttatcttctcttttaaccat
tgaggcaactaagaatattaagttttgttcttcttttgtaaggctatactttcctgcaacaagag
tattagacatagctatttctttgccagcatttacatttttaacttctttcatagaactagagtca
ttatctcgatatacaaattctataaaacttctattagtaaaacaactacttcataaaaaaaagta
gttttaacgatacaaaaagtagttttaaattcaaaaagtgatacaaaaagtagttttaaattcaa
aaagtgatacaaaaagtagttttaaattttttaaaaaagtgcttcaaagccttatgtagcaatac
ttacagaggattaaaaaaaaatctgacaatatataaagagaatatataaagagaatatcttaggg
gattttaaaaaaatcccacagactcaaagacttttttgactttttaaatcctagaaactatactt
taagtacttatttaagtacatggatttagattatgcaaaccgttaattattcaacttttagaaat
gaactatctgattcaatggatagagtaacaaaaaatcatagtcctatgattgtaactagaggttc
aaaaaaagaagcagttgttatgatgtcgttagaggattcttcccttcctttctcgccacgttcgc
cggctttccccgtcaagctctaaatcgggggctccctttagggttccgatttagtgctttacggc
acctcgaccccaaaaaacttgattagggtgatggttcacgtagtgggccatcgccctgatagacg
gtttttcgccctttgacgttggagtccacgttctttaatagtggactcttgttccaaactggaac
aacactcaaccctatctcggtctattcttttgatttataagggattttgccgatttcggcctatt
ggttaaaaaatgagctgatttaacaaaaatttaacgcgaattttaacaaaattcagggcgcaagg
gctgctaaaggaagcggaacacgtagaaagccagtccgcagaaacggtgctgaccccggatgaat
gtcagctactgggctatctggacaagggaaaacgcaagcgcaaagagaaagcaggtagcttgcag
tgggcttacatggcgatagctagactgggcggttttatggacagcaagcgaaccggaattgccag
ctggggcgccctctggtaaggttgggaagccctgcaaagtaaactggatggctttcttgccgcca
aggatctgatggcgcaggggatcaagatctgatcaagagacaggatgaggatcgtttcgcatgat
tgaacaagatggattgcacgcaggttctccggccgcttgggtggagaggctattcggctatgact
gggcacaacagacaatcggctgctctgatgccgccgtgttccggctgtcagcgcaggggcgcccg
gttctttttgtcaagaccgacctgtccggtgccctgaatgaactgcaggacgaggcagcgcggct
atcgtggctggccacgacgggcgttccttgcgcagctgtgctcgacgttgtcactgaagcgggaa
gggactggctgctattgggcgaagtgccggggcaggatctcctgtcatcccaccttgctcctgcc
gagaaagtatccatcatggctgatgcaatgcggcggctgcatacgcttgatccggctacctgccc
attcgaccaccaagcgaaacatcgcatcgagcgagcacgtactcggatggaagccggtcttgtcg
atcaggatgatctggacgaagaAcatcaggggctcgcgccagccgaactgttcgccaggctcaag
gcgcgcatgcccgacggcgaggatctcgtcgtgacccatggcgatgcctgcttgccgaatatcat
ggtggaaaatggccgcttttctggattcatcgactgtggccggctgggtgtggcggaccgctatc
aggacatagcgttggctacccgtgatattgctgaagaActtggcggcgaatgggctgaccgcttc
ctcgtgctttacggtatcgccgctcccgattcgcagcgcatcgccttctatcgccttcttgacga
gttcttctgaactgtcagaccaagtttactcatatatactttagattgatttaaaacttcatttt
taatttaaaaggatctaggtgaagatcctttttgataatctcatgaccaaaatcccttaacgtga
gttttcgttccactgagcgtcagaccccgtagaaaagatcaaaggatcttcttgagatccttttt
ttctgcgcgtaatctgctgcttgcaaacaaaaaaaccaccgctaccagcggtggtttgtttgccg
gatcaagagctaccaactctttttccgaaggtaactggcttcagcagagcgcagataccaaatac
tgttcttctagtgtagccgtagttaggccaccacttcaagaactctgtagcaccgcctacatacc
tcgctctgctaatcctgttaccagtggctgctgccagtggcgataagtcgtgtcttaccgggttg
gactcaagacgatagttaccggataaggcgcagcggtcgggctgaacggggggttcgtgcacaca
gcccagcttggagcgaacgacctacaccgaactgagatacctacagcgtgagctatgagaaagcg
ccacgcttcccgaagggagaaaggcggacaggtatccggtaagcggcagggtcggaacaggagag
cgcacgagggagcttccagggggaaacgcctggtatctttatagtcctgtcgggtttcgccacct
ctgacttgagcgtcgatttttgtgatgctcgtcaggggggcggagcctatggaaaaacgccagca
acgcggcctttttacggttcctggccttttgctggccttttgctcacatgttctttcctgcgtta
tcccctgattctgtggataaccgtattaccgcctttgagtgagctgataccgctcgccgcagccg
aacgaccgagcgcagcgagtcagtgagcgaggaagcggaaAagcgcccaatacgcaaaccgcctc
tccccgcgcgttggccgattcattaatgcagctggcacgacaggtttcccgactggaaagcgggc
agtgagcgcaacgcaattaatgtgagttagctcactcattaggcaccccaggctttacactttat
gcttccggctcgtatgttgtgtggaattgtgagcggataacaatttcacacaggaaacagctatg
accatgattacgccaagctt (SEQ ID NO: 4)
5. PFNLdAp-bfr-N3F8H-SCoV2_(S(2P)) (10224 bp}
ggtacctggttactattgccatcatcacaatattaaaattaattttcttcatttatttttcttaa
atattattattaaaaatagtaaatttaacttatctaaaaatagcataatatcatttttattaaaa
tatctaggttgaattcttagatattttgatatataattagatactaaattgataacttataaaga
attaaattttcttttgtatgctaacttgattgctaatatgaattatactagttagtatgttgatt
ataatgattagagttttaaataatggaggtaacaataggaggtacgtaatggattataaagatca
cgatggtgattacaaagaccatgatatagattataaggatgacgatgataagcatcatcatcacc
accatcatcatggaggtggttcaATGTTTGTGTTTTTAGTTCTTTTACCGTTAGTTTCAAGTCAA
TGTGTGAACTTAACTACACGCACACAACTTCCTCCAGCATATACAAATAGTTTTACTAGAGGTGT
ATATTATCCTGATAAAGTATTCCGTAGTTCTGTTCTACATTCTACACAAGATTTGTTTTTACCGT
TTTTCAGTAATGTCACTTGGTTCCATGCTATTCATGTTTCTGGGACAAACGGTACAAAAAGATTT
GATAACCCTGTTTTACCATTTAATGATGGTGTATATTTTGCTTCAACTGAGAAAAGCAATATAAT
TAGAGGTTGGATTTTCGGAACTACCCTGGATAGCAAGACGCAAAGTTTATTGATCGTAAACAATG
CTACAAACGTCGTAATTAAAGTATGTGAATTTCAATTTTGTAATGACCCTTTTTTAGGAGTCTAT
TATCATAAAAATAATAAATCTTGGATGGAGTCTGAATTTAGAGTTTATTCTAGCGCTAATAACTG
TACATTTGAATATGTTTCACAACCTTTTTTAATGGATCTAGAAGGTAAACAGGGTAATTTTAAAA
ATCTTCGTGAGTTTGTTTTTAAGAACATAGATGGATATTTCAAAATATATTCAAAAGATACTCCT
ATTAATCTAGTTAGAGATCTTCCACAAGGCTTTTCTGCTCTAGAACCATTAGTTGATTTACCAAT
AGGTATAAATATAACTCGTTTCCAAACTTTACTAGCCCTTCACCGTTCGTACTTAACGCCTGGGG
ATTCTTCTAGTGGTTGGACTGCTGGCGCTGCAGCATATTATGTTGGATATCTACAACCTAGAACA
TTTTTATTGAAATACAACGAAAACGGAACTATAACTGACGCTGTTGATTGTGCACTTGATCCATT
AAGTGAGACTAAATGTACTCTAAAAAGTTTTACTGTTGAAAAGGGAATTTATCAAACATCAAATT
TTCGCGTTCAACCAACGGAAAGTATTGTACGTTTTCCGAACATAAGCAATTTATGTCCTTTCGGT
GAGGTATTTAACGCAACTCGTTTTGCGAGCGTATATGCTTGGAATAGAAAAAGAATTAGCAATTG
TGTTGCTGATTATTCGGTCTTATACAATAGTGCTTCGTTTAGCACTTTTAAATGTTACGGAGTAA
GTCCAACAAAGTTAAATGATCTATGTTTCACTAATGTGTATGCTGATTCTTTTGTTATTAGAGGT
GATGAAGTTCGACAAATTGCTCCAGGTCAAACTGGCAAAATTGCGGACTATAATTATAAGCTACC
TGATGATTTTACTGGCTGTGTGATTGCATGGAATAGTAATAATCTAGATTCGAAAGTCGGTGGGA
ATTATAATTATCTTTATAGACTATTTAGAAAATCTAATTTGAAACCATTTGAGAGAGATATATGA
ACAGAAATTTACCAGGCTGGCAGCACACCTTGCAACGGCGTAGAAGGTTTTAATTGTTATTTTCC
ACTACAAAGTTATGGTTTTCAACCAACTAATGGCGTCGGGTATCAACCATATAGAGTTGTCGTAC
TTTCCTTTGAATTACTTCATGCACCAGCTACCGTTTGTGGGCCAAAGAAATCAACTAATCTTGTA
AAGAATAAATGCGTCAATTTTAATTTTAATGGCCTTACAGGCACTGGAGTTTTAACAGAATCCAA
TAAAAAATTTTTACCTTTTCAGCAATTTGGTAGAGATATAGCTGATACTACTGATGCTGTAAGAG
ATCCTCAAACTCTAGAGATTTTAGATATTACCCCGTGTTCATTTGGAGGCGTAAGCGTTATAACT
CCAGGCACGAACACATCAAATCAAGTTGCTGTACTATATCAAGATGTTAATTGCACAGAAGTGCC
TGTTGCCATTCATGCAGATCAACTTACTCCTACATGGCGTGTATATTCTACCGGATCAAATGTAT
TTCAGACTAGAGCTGGTTGTTTAATAGGCGCAGAACATGTAAATAATAGTTATGAGTGTGATATA
CCAATTGGTGCAGGAATATGTGCATCATATCAGACACAGACAAATAGTCCTCGTCGCGCAAGATC
AGTAGCATCACAATCGATTATAGCTTATACAATGTCTTTAGGTGCGGAAAATAGTGTGGCTTATT
CTAATAATTCTATCGCAATCCCTACCAATTTCACTATAAGTGTTACAACCGAAATCTTACCAGTT
AGTATGACAAAGACAAGTGTTGATTGTACTATGTATATATGTGGCGATTCTACTGAGTGTTCTAA
TCTCTTATTACAATATGGTTCGTTTTGTACTCAGTTAAATCGAGCTCTTACAGGTATAGCTGTCG
AGCAAGATAAGAATACCCAGGAAGTCTTTGCACAGGTTAAACAAATTTATAAAACTCCACCAATC
AAAGATTTTGGTGGGTTTAACTTTTCTCAAATACTACCTGATCCATCTAAACCCTCTAAACGTAG
TTTTATTGAAGATTTACTTTTTAATAAAGTAACTCTAGCTGATGCTGGTTTCATTAAACAATACG
GCGATTGTTTGGGTGATATAGCGGCACGTGATTTAATATGCGCACAGAAATTCAACGGTCTGACA
GTCCTACCTCCATTATTGACAGATGAAATGATTGCTCAATATACATCAGCATTGCTTGCTGGCAC
TATCACGAGTGGATGGACTTTTGGTGCTGGCGCTGCTTTACAAATTCCATTTGCCATGCAAATGG
CTTATAGATTTAATGGTATTGGTGTTACACAAAATGTTTTATATGAGAATCAAAAGTTAATAGCT
AACCAATTTAACTCTGCAATTGGCAAGATTCAGGATTCATTATCTAGTACAGCGAGTGCTTTAGG
TAAACTACAAGATGTAGTGAATCAGAATGCTCAAGCACTCAATACTTTGGTTAAACAATTAAGTT
CAAATTTTGGTGCAATTTCAAGTGTACTAAATGATATTCTAAGTCGCTTAGATCCTCCAGAGGCT
GAAGTACAAATCGATAGACTAATTAGAGGTAGATTAGAGTCATTACAAACTTATGTTAGTCAACA
GTTAATTAGAGCTGCAGAAATAAGAGCATCTGCAAATTTGGCAGCCACTAAGATGAGTGAGTGTG
TCCTTGGACAATCAAAACGTGTAGATTTTTGCGGAAAGGGATATCACTTAATGTCATTTCCGCAA
TCTGCACCTCATGGTGTCGTGTTTCTTCATGTTACTTACGTTCCGGCTCAAGAGAAAAACTTCAC
TACGGCTCCAGCGATTTGTCATGATGGTAAAGCTCATTTTCCTCGTGAGGGTGTATTTGTATCAA
ATGGAACACATTGGTTTGTTACTCAAAGAAATTTTTATGAGCCACAAATAATAACTACAGATAAT
ACTTTTGTTAGCGGTAACTGTGACGTAGTTATAGGAATCGTAAACAACACAGTGTATGATCCATT
ACAACCAGAGTTAGATTCTTTTAAAGAAGAACTTGATAAGTATTTCAAAAATCATACTAGCCCTG
ATGTTGACCTTGGTGACATATCAGGCATAAATGCATCAGTTGTTAATATTCAAAAAGAAATAGAT
AGGCTTAATGAAGTTGCTAAAAATCTTAATGAATCTTTAATAGATCTACAAGAACTTGGAAAATA
CGAACAATATATAAAATGGCCTTGGTATATATGGTTAGGGTTTATTGCTGGTCTTATTGCTATTG
TAATGGTAACTATTATGCTATGTTGTATGACATCATGCTGTAGCTGTCTAAAGGGTTGTTGTAGT
TGTGGTTCATGTTGCAAATTTGATGAAGATGATAGTGAGCCAGTTCTTAAAGGTGTAAAATTGCA
TTACACATGAggttaaggatccactagctcgtttcaaattaccgatgatatcggaccgttccaac
ttaccgaccagttcggcaggtatgtatttgcgtgcattcctatccaaaaaaacatcaagccaaaa
gcttgaaaaaacttacaacacagctcaacagagctagattgtaaaaccctgctttgttaagcaga
acgcaaaaattgaatgacttatagtcatatogcttcgaccctcgtagattagtagccttgagcta
ttaactggttgaaacacttaccaaataaagattaaaagcgataaaaatgaaagataaagcagcta
aaaacagagattttagaaagactattttatcagtgttacaacgcaataaagatggctcttttgct
acgcaagcaaatagaaagtctattctgttgcaggcaactaaagaccttaaaaaggtagggtttag
caaggttacagccgaaaacttoggtaataagcattgctatgcacttagagaccattggagagccc
aaggattagctacagcaacgataaaaaatcgtttagcttgtctaaggtggttaggcgagaaaatg
ggcaaagaactacccgataatcgaaaattagagattgagaacaggaagtatagcgataattcaat
caataaagcccaagaaatcgattttaaggcgatttctgccttaactgataggcaagccctagcaa
tacaattacagcgcgaatttgggcttcgtagagaagaaagtttgaagtttcagcctagttatgca
atcaaagagcataaaatcgagcttaaaagctcttggacaaagggtggaagaccacgagaaatccc
aattttgaatgaaaaacagagagaattgttagaaaaagtaaaagaggtagcaggtaaaggctctc
taattgagagcgaaaagtcttataagcaagcaatggaacatttcacgactcgctgtcaaagagca
gggattaagaatgttcatggctttagacatgcgtatgctcaagatagatataggcaattaacagg
gcgtgagtgtcctaaaaatggtggattaacatctaagcagctaacacctgagcaaaagcaacaag
actatgaagctagaatgactattagtgaagagttaggtcatggtagagaagatgtaacagtcaac
tacttaggcagataaaaagcaatatagctatagaagaaaagaaagctattttacatagtagatcg
actcttcttagggattttatattttttgataaatcatctattttgctagttaaatcatcaaattt
atcatcttgttgtttgactaaatctaagaatctattctcttttttaaaatcgttcatgcaaaccg
cctatagctttcttctttttctgaaattatttgtcttcacaccataattaaattcccatttttat
aagtaaagtcttttaaaagcttgtcagtctcttctctagaaatgtaccaaattttacctatttta
ggatacttttcatgaagTtcttctatttttccccagtcctttaatagtctacctttagagtctcg
taaatagttatctttgtgacaggggcctcttttatcttttttaatgtaactatatgttattccaa
cgtcactattactattatccaaatcttttttagcatgccagtaagaactttcataacttaactct
atctttcgacctctttgatatacaacaataaagctatagccagtagtaacaacctgttttacttt
tgttaaatctattaacttcttatttatttttttatgtttttttgaaaatttaaatatttctatat
tcattcctacacttcctcaaatccaaatggtagcttatgattctcttctggtttcttttctaatt
tttttatatttgcaataaaaactctttttctatctttgatttttttattgtcccaattcctccaa
gtatcatcacaaaccctttcaatatcatgtaaatgatgatgtctaaatattgatctgacataata
cagatctaggtctagttcatcacttaacacaacttctctaagtctttcagatgcttcgattggta
tgtaatcctctttatttttagtatctaaaagcttttgcttaaattcttcttctgtctctgctacc
ttactaactgtaaacttgatatttgtaatcttacgaccatgttttctgtgatgatccttgtcatc
ataggttacaaaaatatccgataattgattaatctcttctagtgctggtaataggaacttatttt
taaaatttgaatatctgttgctgtaacttttaggtaaatcaaaatcattaatcatatcatcgaca
tacaatacgcaatcaactatattagcataccctgcttgttogcctaatttgcttttgagaagtaa
gtataatctgcttgaatacttacttttaaatgaaaatagtaactgtctttctgctttagtaaagt
actcttgtagttgtatcatgtgtggcattaatgaccaatgaaactcgcaaattaaagcactgctt
ttagggtctgcttcaatatatgcaaaccagttagctatcttcgtttgttctttattcagccatac
tggcttagacattattgagtgcattaattgcttcaatctcactctgttatgcttaacccctgtag
ctttttcaagatcagataggcttatcttatacctgtgaaactctttatcttctcttttaaccatt
gaggcaactaagaatattaagttttgttcttcttttgtaaggctatactttcctgcaacaagagt
attagacatagctatttctttgccagcatttacatttttaacttctttcatagaactagagtcat
tatctcgatatacaaattctataaaacttctattagtaaaacaactacttcataaaaaaaagtag
ttttaacgatacaaaaagtagttttaaattcaaaaagtgatacaaaaagtagttttaaattcaaa
aagtgatacaaaaagtagttttaaattttttaaaaaagtgcttcaaagccttatgtagcaatact
tacagaggattaaaaaaaaatctgacaatatataaagagaatatataaagagaatatcttagggg
attttaaaaaaatcccacagactcaaagacttttttgactttttaaatcctagaaactatacttt
aagtacttatttaagtacatggatttagattatgcaaaccgttaattattcaacttttagaaatg
aactatctgattcaatggatagagtaacaaaaaatcatagtcctatgattgtaactagaggttca
aaaaaagaagcagttgttatgatgtcgttagaggattcttcccttcctttctogccacgttcgcc
ggctttccccgtcaagctctaaatcgggggctccctttagggttccgatttagtgctttacggca
cctcgaccccaaaaaacttgattagggtgatggttcacgtagtgggccatcgccctgatagacgg
tttttcgccctttgacgttggagtccacgttctttaatagtggactcttgttccaaactggaaca
acactcaaccctatctcggtctattcttttgatttataagggattttgccgatttcggcctattg
gttaaaaaatgagctgatttaacaaaaatttaacgcgaattttaacaaaattcagggogcaaggg
ctgctaaaggaagcggaacacgtagaaagccagtccgcagaaacggtgctgaccccggatgaatg
tcagctactgggctatctggacaagggaaaacgcaagcgcaaagagaaagcaggtagc11gcagt
gggcttacatggcgatagctagactgggcggttttatggacagcaagcgaaccggaattgccagc
tggggcgccctctggtaaggttgggaagccctgcaaagtaaactggatggctttcttgccgccaa
ggatctgatggcgcaggggatcaagatctgatcaagagacaggatgaggatcgtttcgcatgatt
gaacaagatggattgcacgcaggttctccggccgcttgggtggagaggctattcggctatgactg
ggcacaacagacaatcggctgctctgatgccgccgtgttccggctgtcagcgcaggggcgcccgg
ttctttttgtcaagaccgacctgtccggtgccctgaatgaactgcaggacgaggcagcgcggcta
tcgtggctggccacgacgggcgttccttgcgcagctgtgctcgacgttgtcactgaagcgggaag
ggactggctgctattgggcgaagtgccggggcaggatctcctgtcatcccaccttgctcctgccg
agaaagtatccatcatggctgatgcaatgcggcggctgcatacgcttgatccggctacctgccca
ttcgaccaccaagcgaaacatcgcatcgagcgagcacgtactcggatggaagccggtcttgtcga
tcaggatgatctggacgaagaAcatcaggggctcgcgccagccgaactgttcgccaggctcaagg
cgcgcatgcccgacggcgaggatctcgtcgtgacccatggcgatgcctgcttgccgaatatcatg
gtggaaaatggccgcttttctggattcatcgactgtggccggctgggtgtggcggaccgctatca
ggacatagcgttggctacccgtgatattgctgaagaActtggcggcgaatgggctgaccgcttcc
tcgtgctttacggtatcgccgctcccgattcgcagcgcatcgccttctatcgccttcttgacgag
ttcttctgaactgtcagaccaagtttactcatatatactttagattgatttaaaacttcattttt
aatttaaaaggatctaggtgaagatcctttttgataatctcatgaccaaaatcccttaacgtgag
ttttcgttccactgagcgtcagaccccgtagaaaagatcaaaggatcttcttgagatcctttttt
tctgcgcgtaatctgctgcttgcaaacaaaaaaaccaccgctaccagcggtggtttgtttgccgg
atcaagagctaccaactctttttccgaaggtaactggcttcagcagagcgcagataccaaatact
gttcttctagtgtagccgtagttaggccaccacttcaagaactctgtagcaccgcctacatacct
cgctctgctaatcctgttaccagtggctgctgccagtggcgataagtcgtgtcttaccgggttgg
actcaagacgatagttaccggataaggcgcagcggtcgggctgaacggggggttcgtgcacacag
cccagcttggagcgaacgacctacaccgaactgagatacctacagcgtgagctatgagaaagcgc
cacgcttcccgaagggagaaaggcggacaggtatccggtaagcggcagggtcggaacaggagagc
gcacgagggagcttccagggggaaacgcctggtatctttatagtcctgtcgggtttcgccacctc
tgacttgagcgtcgatttttgtgatgctcgtcaggggggcggagcctatggaaaaacgccagcaa
cgcggcctttttacggttcctggccttttgctggccttttgctcacatgttctttcctgcgttat
cccctgattctgtggataaccgtattaccgcctttgagtgagctgataccgctcgccgcagccga
acgaccgagcgcagcgagtcagtgagcgaggaagcggaaAagcgcccaatacgcaaaccgcctct
ccccgcgcgttggccgattcattaatgcagctggcacgacaggtttcccgactggaaagcgggca
gtgagcgcaacgcaattaatgtgagttagctcactcattaggcaccccaggctttacactttatg
cttccggctcgtatgttgtgtggaattgtgagcggataacaatttcacacaggaaacagctatga
ccatgattacgccaagctt (SEQ ID NO: 5)
6. pFNLdAp-bfr-N3F8H-SCoV2_(S1) (8460 bp)
TGAggttaaggatccactagctcgtttcaaattaccgatgatatcggaccgttccaacttaccga
ccagttcggcaggtatgtatttgcgtgcattcctatccaaaaaaacatcaagccaaaagcttgaa
aaaacttacaacacagctcaacagagctagattgtaaaaccctgctttgttaagcagaacgcaaa
aattgaatgacttatagtcatatcgcttcgaccctcgtagattagtagccttgagctattaactg
gttgaaacacttaccaaataaagattaaaagcgataaaaatgaaagataaagcagctaaaaacag
agattttagaaagactattttatcagtgttacaacgcaataaagatggctcttttgctacgcaag
caaatagaaagtctattctgttgcaggcaactaaagaccttaaaaaggtagggtttagcaaggtt
acagccgaaaacttcggtaataagcattgctatgcacttagagaccattggagagcccaaggatt
agctacagcaacgataaaaaatcgtttagcttgtctaaggtggttaggcgagaaaatgggcaaag
aactacccgataatcgaaaattagagattgagaacaggaagtatagcgataattcaatcaataaa
gcccaagaaatcgattttaaggcgatttctgccttaactgataggcaagccctagcaatacaatt
acagcgcgaatttgggcttcgtagagaagaaagtttgaagtttcagcctagttatgcaatcaaag
agcataaaatcgagcttaaaagctcttggacaaagggtggaagaccacgagaaatcccaattttg
aatgaaaaacagagagaattgttagaaaaagtaaaagaggtagcaggtaaaggctctctaattga
gagcgaaaagtcttataagcaagcaatggaacatttcacgactcgctgtcaaagagcagggatta
agaatgttcatggctttagacatgcgtatgctcaagatagatataggcaattaacagggcgtgag
tgtcctaaaaatggtggattaacatctaagcagctaacacctgagcaaaagcaacaagactatga
agctagaatgactattagtgaagagttaggtcatggtagagaagatgtaacagtcaactacttag
gcagataaaaagcaatatagctatagaagaaaagaaagctattttacatagtagatcgactcttc
ttagggattttatattttttgataaatcatctattttgctagttaaatcatcaaatttatcatct
tgttgtttgactaaatctaagaatctattctcttttttaaaatcgttcatgcaaaccgcctatag
ctttcttctttttctgaaattatttgtcttcacaccataattaaattcccatttttataagtaaa
gtcttttaaaagcttgtcagtctcttctctagaaatgtaccaaattttacctattttaggatact
tttcatgaagTtcttctatttttccccagtcctttaatagtctacctttagagtctcgtaaatag
ttatctttgtgacaggggcctcttttatcttttttaatgtaactatatgttattccaacgtcact
attactattatccaaatcttttttagcatgccagtaagaactttcataacttaactctatctttc
gacctctttgatatacaacaataaagctatagccagtagtaacaacctgttttacttttgttaaa
tctattaacttcttatttatttttttatgtttttttgaaaatttaaatatttctatattcattcc
tacacttcctcaaatccaaatggtagcttatgattctcttctggtttcttttctaatttttttat
atttgcaataaaaactctttttctatctttgatttttttattgtcccaattcctccaagtatcat
cacaaaccctttcaatatcatgtaaatgatgatgtctaaatattgatctgacataatacagatct
aggtctagttcatcacttaacacaacttctctaagtctttcagatgcttcgattggtatgtaatc
ctctttatttttagtatctaaaagcttttgcttaaattcttcttctgtctctgctaccttactaa
ctgtaaacttgatatttgtaatcttacgaccatgttttctgtgatgatccttgtcatcataggtt
acaaaaatatccgataattgattaatctcttctagtgctggtaataggaacttatttttaaaatt
tgaatatctgttgctgtaacttttaggtaaatcaaaatcattaatcatatcatcgacatacaata
cgcaatcaactatattagcataccctgcttgttcgcctaatttgcttttgagaagtaagtataat
ctgcttgaatacttacttttaaatgaaaatagtaactgtctttctgctttagtaaagtactcttg
tagttgtatcatgtgtggcattaatgaccaatgaaactcgcaaattaaagcactgcttttagggt
ctgcttcaatatatgcaaaccagttagctatcttogtttgttctttattcagccatactggctta
gacattattgagtgcattaattgcttcaatctcactctgttatgcttaacccctgtagctttttc
aagatcagataggcttatcttatacctgtgaaactctttatcttctcttttaaccattgaggcaa
ctaagaatattaagttttgttcttcttttgtaaggctatactttcctgcaacaagagtattagac
atagctatttctttgccagcatttacatttttaacttctttcatagaactagagtcattatctcg
atatacaaattctataaaacttctattagtaaaacaactacttcataaaaaaaagtagttttaac
gatacaaaaagtagttttaaattcaaaaagtgatacaaaaagtagttttaaattcaaaaagtgat
acaaaaagtagttttaaattttttaaaaaagtgcttcaaagccttatgtagcaatacttacagag
gattaaaaaaaaatctgacaatatataaagagaatatataaagagaatatcttaggggattttaa
aaaaatcccacagactcaaagacttttttgactttttaaatcctagaaactatactttaagtact
tatttaagtacatggatttagattatgcaaaccgttaattattcaacttttagaaatgaactatc
tgattcaatggatagagtaacaaaaaatcatagtcctatgattgtaactagaggttcaaaaaaag
aagcagttgttatgatgtcgttagaggattcttcccttcctttctogccacgttcgccggctttc
cccgtcaagctctaaatcgggggctccctttagggttccgatttagtgctttacggcacctcgac
cccaaaaaacttgattagggtgatggttcacgtagtgggccatcgccctgatagacggtttttog
ccctttgacgttggagtccacgttctttaatagtggactcttgttccaaactggaacaacactca
accctatctcggtctattcttttgatttataagggattttgccgatttoggcctattggttaaaa
aatgagctgatttaacaaaaatttaacgcgaattttaacaaaattcagggcgcaagggctgctaa
aggaagcggaacacgtagaaagccagtccgcagaaacggtgctgaccccggatgaatgtcagcta
ctgggctatctggacaagggaaaacgcaagcgcaaagagaaagcaggtagcttgcagtgggctta
catggcgatagctagactgggcggttttatggacagcaagcgaaccggaattgccagctggggcg
ccctctggtaaggttgggaagccctgcaaagtaaactggatggctttcttgccgccaaggatctg
atggcgcaggggatcaagatctgatcaagagacaggatgaggatcgtttcgcatgattgaacaag
atggattgcacgcaggttctccggccgcttgggtggagaggctattcggctatgactgggcacaa
cagacaatcggctgctctgatgccgccgtgttccggctgtcagcgcaggggcgcccggttctttt
tgtcaagaccgacctgtccggtgccctgaatgaactgcaggacgaggcagcgcggctatcgtggc
tggccacgacgggcgttccttgcgcagctgtgctcgacgttgtcactgaagcgggaagggactgg
ctgctattgggcgaagtgccggggcaggatctcctgtcatcccaccttgctcctgccgagaaagt
atccatcatggctgatgcaatgcggcggctgcatacgcttgatccggctacctgcccattcgacc
accaagcgaaacatcgcatcgagcgagcacgtactcggatggaagccggtcttgtcgatcaggat
gatctggacgaagaAcatcaggggctcgcgccagccgaactgttcgccaggctcaaggcgcgcat
gcccgacggcgaggatctcgtcgtgacccatggcgatgcctgcttgccgaatatcatggtggaaa
atggccgcttttctggattcatcgactgtggccggctgggtgtggcggaccgctatcaggacata
gcgttggctacccgtgatattgctgaagaActtggcggcgaatgggctgaccgcttcctcgtgct
ttacggtatcgccgctcccgattcgcagcgcatcgccttctatcgccttcttgacgagttcttct
gaactgtcagaccaagtttactcatatatactttagattgatttaaaacttcatttttaatttaa
aaggatctaggtgaagatcctttttgataatctcatgaccaaaatcccttaacgtgagttttcgt
tccactgagcgtcagaccccgtagaaaagatcaaaggatcttcttgagatcctttttttctgcgc
gtaatctgctgcttgcaaacaaaaaaaccaccgctaccagcggtggtttgtttgccggatcaaga
gctaccaactctttttccgaaggtaactggcttcagcagagcgcagataccaaatactgttcttc
tagtgtagccgtagttaggccaccacttcaagaactctgtagcaccgcctacatacctcgctctg
ctaatcctgttaccagtggctgctgccagtggcgataagtcgtgtcttaccgggttggactcaag
acgatagttaccggataaggcgcagcggtcgggctgaacggggggttcgtgcacacagcccagct
tggagcgaacgacctacaccgaactgagatacctacagcgtgagctatgagaaagcgccacgctt
cccgaagggagaaaggcggacaggtatccggtaagcggcagggtcggaacaggagagcgcacgag
ggagcttccagggggaaacgcctggtatctttatagtcctgtcgggtttcgccacctctgacttg
agcgtcgatttttgtgatgctcgtcaggggggcggagcctatggaaaaacgccagcaacgcggcc
tttttacggttcctggccttttgctggccttttgctcacatgttctttcctgcgttatcccctga
ttctgtggataaccgtattaccgcctttgagtgagctgataccgctcgccgcagccgaacgaccg
agcgcagcgagtcagtgagcgaggaagcggaaAagcgcccaatacgcaaaccgcctctccccgcg
cgttggccgattcattaatgcagctggcacgacaggtttcccgactggaaagcgggcagtgagcg
caacgcaattaatgtgagttagctcactcattaggcaccccaggctttacactttatgcttccgg
ctcgtatgttgtgtggaattgtgagcggataacaatttcacacaggaaacagctatgaccatgat
tacgccaagcttggtacctggttactattgccatcatcacaatattaaaattaattttcttcatt
tatttttcttaaatattattattaaaaatagtaaatttaacttatctaaaaatagcataatatca
tttttattaaaatatctaggttgaattcttagatattttgatatataattagatactaaattgat
aacttataaagaattaaattttcttttgtatgctaacttgattgctaatatgaattatactagtt
agtatgttgattataatgattagagttttaaataatggaggtaacaataggaggtacgtaatgga
ttataaagatcacgatggtgattacaaagaccatgatatagattataaggatgacgatgataagc
atcatcatcaccaccatcatcatggaggtggttcaATGTTTGTGTTTTTAGTTCTTTTACCGTTA
GTTTCAAGTCAATGTGTGAACTTAACTACACGCACACAACTTCCTCCAGCATATACAAATAGTTT
TACTAGAGGTGTATATTATCCTGATAAAGTATTCCGTAGTTCTGTTCTACATTCTACACAAGATT
TGTTTTTACCGTTTTTCAGTAATGTCACTTGGTTCCATGCTATTCATGTTTCTGGGACAAACGGT
ACAAAAAGATTTGATAACCCTGTTTTACCATTTAATGATGGTGTATATTTTGCTTCAACTGAGAA
AAGCAATATAATTAGAGGTTGGATTTTCGGAACTACCCTGGATAGCAAGACGCAAAGTTTATTGA
TCGTAAACAATGCTACAAACGTCGTAATTAAAGTATGTGAATTTCAATTTTGTAATGACCCTTTT
TTAGGAGTCTATTATCATAAAAATAATAAATCTTGGATGGAGTCTGAATTTAGAGTTTATTCTAG
CGCTAATAACTGTACATTTGAATATGTTTCACAACCTTTTTTAATGGATCTAGAAGGTAAACAGG
GTAATTTTAAAAATCTTCGTGAGTTTGTTTTTAAGAACATAGATGGATATTTCAAAATATATTCA
AAACATACTCCTATTAATCTAGTTAGAGATCTTCCACAAGGCTTTTCTGCTCTAGAACCATTAGT
TGATTTACCAATAGGTATAAATATAACTCGTTTCCAAACTTTACTAGCCCTTCACCGTTCGTACT
TAACGCCTGGGGATTCTTCTAGTGGTTGGACTGCTGGCGCTGCAGCATATTATGTTGGATATCTA
CAACCTAGAACATTTTTATTGAAATACAACGAAAACGGAACTATAACTGACGCTGTTGATTGTGC
ACTTGATCCATTAAGTGAGACTAA+TGTACTCTAAAA+GTTTTACTGTTGAAA+GGGAATTTATC
AAACATCAAATTTTCGCGTTCAACCAACGGAAAGTATTGTACGTTTTCCGAACATAACCAATTTA
TGTCCTTTCGGTGAGGTATTTAACGCAACTCGTTTTGCGAGCGTATATGCTTGGAATAGAAAAAG
AATTAGCAATTGTGTTGCTGATTATTCGGTCTTATACAATAGTGCTTCGTTTAGCACTTTTAAAT
GTTACGGAGTAAGTCCAACAAAGTTAAATGATCTATGTTTCACTAATGTGTATGCTGATTCTTTT
GTTATTAGAGGTGATGAAGTTCGACAAATTGCTCCAGGTCAAACTGGCAAAATTGCGGACTATAA
TTATAAGCTACCTGATGATTTTACTGGCTGTGTGATTGCATGGAATAGTAATAATCTAGATTCGA
AAGTCGGTGGGAATTATAATTATCTTTATAGACTATTTAGAAAATCTAATTTGAAACCATTTGAG
AGAGATATATCAACAGAAATTTACCAGGCTGGCAGCACACCTTGCAACGGCGTAGAAGGTTTTAA
TTGTTATTTTCCACTACAAAGTTATGGTTTTCAACCAACTAATGGCGTCGGGTATCAACCATATA
GAGTTGTCGTACTTTCCTTTGAATTACTTCATGCACCAGCTACCGTTTGTGGGCCAAAGAAATCA
ACTAATCTTGTAAAGAATAAATGCGTCAATTTTAATTTTAATGGCCTTACAGGCACTGGAGTTTT
AACAGAATCCAATAAAAAATTTTTACCTTTTCAGCAATTTGGTAGAGATATAGCTGATACTACTG
ATGCTGTAAGAGATCCTCAAACTCTAGAGATTTTAGATATTACCCCGTGTTCATTTGGAGGCGTA
AGCGTTATAACTCCAGGCACGAACACATCAAATCAAGTTGCTGTACTATATCAAGATGTTAATTG
CACAGAAGTGCCTGTTGCCATTCATGCAGATCAACTTACTCCTACATGGCGTGTATATTCTACCG
GATCAAATGTATTTCAGACTAGAGCTGGTTGTTTAATAGGCGCAGAACATGTAAATAATAGTTAT
GAGTGTGATATACCAATTGGTGCAGGAATATGTGCATCATATCAGACACAGACAAATAGTCCTCG
TCGCGCAAGA (SEQ ID NO: 6)
7. pFNLdAp-bfr-N3F8H-SCov2_(S2) (8067 bp)
TCAGTAGCATCACAATCGATTATAGCTTATACAATGTCTTTAGGTGCGGAAAATAGTGTGGCTTA
TTCTAATAATTCTATCGCAATCCCTACCAATTTCACTATAAGTGTTACAACCGAAATCTTACCAG
TTAGTATGACAAAGACAAGTGTTGATTGTACTATGTATATATGTGGCGATTCTACTGAGTGTTCT
AATCTCTTATTACAATATGGTTCGTTTTGTACTCAGTTAAATCGAGCTCTTACAGGTATAGCTGT
CGAGCAAGATAAGAATACCCAGGAAGTCTTTGCACAGGTTAAACAAATTTATAAAACTCCACCAA
TCAAAGATTTTGGTGGGTTTAACTTTTCTCAAATACTACCTGATCCATCTAAACCCTCTAAACGT
AGTTTTATTGAAGATTTACTTTTTAATAAAGTAACTCTAGCTGATGCTGGTTTCATTAAACAATA
CGGCGATTGTTTGGGTGATATAGCGGCACGTGATTTAATATGCGCACAGAAATTCAACGGTCTGA
CAGTCCTACCTCCATTATTGACAGATGAAATGATTGCTCAATATACATCAGCATTGCTTGCTGGC
ACTATCACGAGTGGATGGACTTTTGGTGCTGGCGCTGCTTTACAAATTCCATTTGCCATGCAAAT
GGCTTATAGATTTAATGGTATTGGTGTTACACAAAATGTTTTATATGAGAATCAAAAGTTAATAG
CTAACCAATTTAACTCTGCAATTGGCAAGATTCAGGATTCATTATCTAGTACAGCGAGTGCTTTA
GGTAAACTACAAGATGTAGTGAATCAGAATGCTCAAGCACTCAATACTTTGGTTAAACAATTAAG
TTCAAATTTTGGTGCAATTTCAAGTGTACTAAATGATATTCTAAGTCGCTTAGATCCTCCAGAGG
CTGAAGTACAAATCGATAGACTAATTACAGGTAGATTACAGTCATTACAAACTTATGTTACTCAA
CAGTTAATTAGAGCTGCAGAAATAAGAGCATCTGCAAATTTGGCAGCCACTAAGATGAGTGAGTG
TGTCCTTGGACAATCAAAACGTGTAGATTTTTGCGGAAAGGGATATCACTTAATGTCATTTCCGC
AATCTGCACCTCATGGTGTCGTGTTTCTTCATGTTACTTACGTTCCGGCTCAAGAGAAAAACTTC
ACTACGGCTCCAGCGATTTGTCATGATGGTAAAGCTCATTTTCCTCGTGAGGGTGTATTTGTATC
AAATGGAACACATTGGTTTGTTACTCAAAGAAATTTTTATGAGCCACAAATAATAACTACAGATA
ATACTTTTGTTAGCGGTAACTGTGACGTAGTTATAGGAATCGTAAACAACACAGTGTATGATCCA
TTACAACCAGAGTTAGATTCTTTTAAAGAAGAACTTGATAAGTATTTCAAAAATCATACTAGCCC
TGATGTTGACCTTGGTGACATATCAGGCATAAATGCATCAGTTGTTAATATTCAAAAAGAAATAG
ATAGGCTTAATGAAGTTGCTAAAAATCTTAATGAATCTTTAATAGATCTACAAGAACTTGGAAAA
TACGAACAATATATAAAATGGCCTTGGTATATATGGTTAGGGTTTATTGCTGGTCTTATTGCTAT
TGTAATGGTAACTATTATGCTATGTTGTATGACATCATGCTGTAGCTGTCTAAAGGGTTGTTGTA
GTTGTGGTTCATGTTGCAAATTTGATGAAGATGATAGTGAGCCAGTTCTTAAAGGTGTAAAATTG
CATTACACATGAggttaaggatccactagctcgtttcaaattaccgatgatatcggaccgttcca
acttaccgaccagttcggcaggtatgtatttgcgtgcattcctatccaaaaaaacatcaagccaa
aagcttgaaaaaacttacaacacagctcaacagagctagattgtaaaaccctgctttgttaagca
gaacgcaaaaattgaatgacttatagtcatategcttcgaccctcgtagattagtagccttgagc
tattaactggttgaaacacttaccaaataaagattaaaagcgataaaaatgaaagataaagcagc
taaaaacagagattttagaaagactattttatcagtgttacaacgcaataaagatggctcttttg
ctacgcaagcaaatagaaagtctattctgttgcaggcaactaaagaccttaaaaaggtagggttt
agcaaggttacagccgaaaacttcggtaataagcattgctatgcacttagagaccattggagagc
ccaaggattagctacagcaacgataaaaaatcgtttagcttgtctaaggtggttaggcgagaaaa
tgggcaaagaactacccgataatcgaaaattagagattgagaacaggaagtatagcgataattca
atcaataaagcccaagaaatcgattttaaggcgatttctgccttaactgataggcaagccctagc
aatacaattacagcgcgaatttgggcttcgtagagaagaaagtttgaagtttcagcctagttatg
caatcaaagagcataaaatcgagcttaaaagctcttggacaaagggtggaagaccacgagaaatc
ccaattttgaatgaaaaacagagagaattgttagaaaaagtaaaagaggtagcaggtaaaggctc
tctaattgagagcgaaaagtcttataagcaagcaatggaacatttcacgactcgctgtcaaagag
cagggattaagaatgttcatggctttagacatgcgtatgctcaagatagatataggcaattaaca
gggcgtgagtgtcctaaaaatggtggattaacatctaagcagctaacacctgagcaaaagcaaca
agactatgaagctagaatgactattagtgaagagttaggtcatggtagagaagatgtaacagtca
actacttaggcagataaaaagcaatatagctatagaagaaaagaaagctattttacatagtagat
cgactcttcttagggattttatattttttgataaatcatctattttgctagttaaatcatcaaat
ttatcatcttgttgtttgactaaatctaagaatctattctcttttttaaaatcgttcatgcaaac
cgcctatagctttcttctttttctgaaattatttgtcttcacaccataattaaattcccattttt
ataagtaaagtcttttaaaagcttgtcagtctcttctctagaaatgtaccaaattttacctattt
taggatacttttcatgaagTtcttctatttttccccagtcctttaatagtctacctttagagtct
cgtaaatagttatctttgtgacaggggcctcttttatcttttttaatgtaactatatgttattcc
aacgtcactattactattatccaaatcttttttagcatgccagtaagaactttcataacttaact
ctatctttcgacctctttgatatacaacaataaagctatagccagtagtaacaacctgttttact
tttgttaaatctattaacttcttatttatttttttatgtttttttgaaaatttaaatatttctat
attcattcctacacttcctcaaatccaaatggtagcttatgattctcttctggtttcttttctaa
tttttttatatttgcaataaaaactctttttctatctttgatttttttattgtcccaattcctcc
aagtatcatcacaaaccctttcaatatcatgtaaatgatgatgtctaaatattgatctgacataa
tacagatctaggtctagttcatcacttaacacaacttctctaagtctttcagatgcttcgattgg
tatgtaatcctctttatttttagtatctaaaagcttttgcttaaattcttcttctgtctctgcta
ccttactaactgtaaacttgatatttgtaatcttacgaccatgttttctgtgatgatccttgtca
tcataggttacaaaaatatccgataattgattaatctcttctagtgctggtaataggaacttatt
tttaaaatttgaatatctgttgctgtaacttttaggtaaatcaaaatcattaatcatatcatcga
catacaatacgcaatcaactatattagcataccctgcttgttcgcctaatttgcttttgagaagt
aagtataatctgcttgaatacttacttttaaatgaaaatagtaactgtctttctgctttagtaaa
gtactcttgtagttgtatcatgtgtggcattaatgaccaatgaaactcgcaaattaaagcactgc
ttttagggtctgcttcaatatatgcaaaccagttagctatcttcgtttgttctttattcagccat
actggcttagacattattgagtgcattaattgcttcaatctcactctgttatgcttaacccctgt
agctttttcaagatcagataggcttatcttatacctgtgaaactctttatcttctcttttaacca
ttgaggcaactaagaatattaagttttgttcttcttttgtaaggctatactttcctgcaacaaga
gtattagacatagctatttctttgccagcatttacatttttaacttctttcatagaactagagtc
attatctcgatatacaaattctataaaacttctattagtaaaacaactacttcataaaaaaaagt
agttttaacgatacaaaaagtagttttaaattcaaaaagtgatacaaaaagtagttttaaattca
aaaagtgatacaaaaagtagttttaaattttttaaaaaagtgcttcaaagccttatgtagcaata
cttacagaggattaaaaaaaaatctgacaatatataaagagaatatataaagagaatatcttagg
ggattttaaaaaaatcccacagactcaaagacttttttgactttttaaatcctagaaactatact
ttaagtacttatttaagtacatggatttagattatgcaaaccgttaattattcaacttttagaaa
tgaactatctgattcaatggatagagtaacaaaaaatcatagtcctatgattgtaactagaggtt
caaaaaaagaagcagttgttatgatgtcgttagaggattcttcccttcctttctegccacgttcg
ccggctttccccgtcaagctctaaatcgggggctccctttagggttccgatttagtgctttacgg
cacctcgaccccaaaaaacttgattagggtgatggttcacgtagtgggccategccctgatagac
ggtttttcgccctttgacgttggagtccacgttctttaatagtggactcttgttccaaactggaa
caacactcaaccctatcteggtctattcttttgatttataagggattttgccgatttcggcctat
tggttaaaaaatgagctgatttaacaaaaatttaacgcgaattttaacaaaattcagggcgcaag
ggctgctaaaggaagcggaacacgtagaaagccagtccgcagaaacggtgctgaccccggatgaa
tgtcagctactgggctatctggacaagggaaaacgcaagcgcaaagagaaagcaggtagcttgca
gtgggcttacatggcgatagctagactgggcggttttatggacagcaagcgaaccggaattgcca
gctggggcgccctctggtaaggttgggaagccctgcaaagtaaactggatggctttcttgccgcc
aaggatctgatggcgcaggggatcaagatctgatcaagagacaggatgaggatcgtttcgcatga
ttgaacaagatggattgcacgcaggttctccggccgcttgggtggagaggctattcggctatgac
tgggcacaacagacaatcggctgctctgatgccgccgtgttccggctgtcagcgcaggggcgccc
ggttctttttgtcaagaccgacctgtccggtgccctgaatgaactgcaggacgaggcagcgcggc
tategtggctggccacgacgggcgttccttgcgcagctgtgctcgacgttgtcactgaagcggga
agggactggctgctattgggcgaagtgccggggcaggatctcctgtcatcccaccttgctcctgc
cgagaaagtatccatcatggctgatgcaatgcggcggctgcatacgcttgatccggctacctgcc
cattcgaccaccaagcgaaacatcgcatcgagcgagcacgtactcggatggaagccggtcttgtc
gatcaggatgatctggacgaagaAcatcaggggctcgcgccagccgaactgttcgccaggctcaa
ggcgcgcatgcccgacggcgaggatctcgtcgtgacccatggcgatgcctgcttgccgaatatca
tggtggaaaatggccgcttttctggattcatcgactgtggccggctgggtgtggcggaccgctat
caggacatagcgttggctacccgtgatattgctgaagaActtggcggcgaatgggctgaccgctt
cctcgtgctttacggtatcgccgctcccgattcgcagcgcatcgccttctatcgccttcttgacg
agttcttctgaactgtcagaccaagtttactcatatatactttagattgatttaaaacttcattt
ttaatttaaaaggatctaggtgaagatcctttttgataatctcatgaccaaaatcccttaacgtg
agttttcgttccactgagcgtcagaccccgtagaaaagatcaaaggatcttcttgagatcctttt
tttctgcgcgtaatctgctgcttgcaaacaaaaaaaccaccgctaccagcggtggtttgtttgcc
ggatcaagagctaccaactctttttccgaaggtaactggcttcagcagagcgcagataccaaata
ctgttcttctagtgtagccgtagttaggccaccacttcaagaactctgtagcaccgcctacatac
ctcgctctgctaatcctgttaccagtggctgctgccagtggcgataagtcgtgtcttaccgggtt
ggactcaagacgatagttaccggataaggcgcagcggtcgggctgaacggggggttcgtgcacac
agcccagcttggagcgaacgacctacaccgaactgagatacctacagcgtgagctatgagaaagc
gccacgcttcccgaagggagaaaggcggacaggtatccggtaagcggcagggtcggaacaggaga
gcgcacgagggagcttccagggggaaacgcctggtatctttatagtcctgtcgggtttcgccacc
tctgacttgagcgtcgatttttgtgatgctcgtcaggggggcggagcctatggaaaaacgccagc
aacgcggcctttttacggttcctggccttttgctggccttttgctcacatgttctttcctgcgtt
atcccctgattctgtggataaccgtattaccgcctttgagtgagctgataccgctcgccgcagcc
gaacgaccgagcgcagcgagtcagtgagcgaggaagcggaaAagcgcccaatacgcaaaccgcct
ctccccgcgcgttggccgattcattaatgcagctggcacgacaggtttcccgactggaaagcggg
cagtgagcgcaacgcaattaatgtgagttagctcactcattaggcaccccaggctttacacttta
tgcttccggctcgtatgttgtgtggaattgtgagcggataacaatttcacacaggaaacagctat
gaccatgattacgccaagcttggtacctggttactattgccatcatcacaatattaaaattaatt
ttcttcatttatttttcttaaatattattattaaaaatagtaaatttaacttatctaaaaatagc
ataatatcatttttattaaaatatctaggttgaattcttagatattttgatatataattagatac
taaattgataacttataaagaattaaattttcttttgtatgctaacttgattgctaatatgaatt
atactagttagtatgttgattataatgattagagttttaaataatggaggtaacaataggaggta
cgtaatg (SEQ ID NO: 7)
8. pFNLdAp-bfr-N3F8H-SCoV2_(S2E) (8400 bp)
ggtacctggttactattgccatcatcacaatattaaaattaattttcttcatttatttttcttaa
atattattattaaaaatagtaaatttaacttatctaaaaatagcataatatcatttttattaaaa
tatctaggttgaattcttagatattttgatatataattagatactaaattgataacttataaaga
attaaattttcttttatatactaacttaattactaatataaattatactaattaatatattaatt
ataatgattagagttttaaataatggaggtaacaataggaggtacgtaatggattataaagatca
cgatggtgattacaaagaccatgatatagattataaggatgacgatgataagcatcatcatcacc
accatcatcatggaggtggttcaATGTCTGTAGCGAGTCAATCAATAATAGCATATACAATGTCA
TTAGGCGCAGAAAATAGTGTGGCTTATTCTAATAATTCTATCGCAATCCCTACCAATTTCACTAT
AAGTGTTACAACCGAAATCTTACCAGTTAGTATGACAAAGACAAGTGTTGATTGTACTATGTATA
TATGTGGCGATTCTACTGAGTGTTCTAATCTCTTATTACAATATGGTTCGTTTTGTACTCAGTTA
AATCGAGCTCTTACAGGTATAGCTGTCGAGCAAGATAAGAATACCCAGGAAGTCTTTGCACAGGT
TAAACAAATTTATAAAACTCCACCAATCAAAGATTTTGGTGGGTTTAACTTTTCTCAAATACTAC
CTGATCCATCTAAACCCTCTAAACGTAGTTTTATTGAAGATTTACTTTTTAATAAAGTAACTCTA
GCTGATGCTGGTTTCATTAAACAATACGGCGATTGTTTGGGTGATATAGCGGCACGTGATTTAAT
ATGCGCACAGAAATTCAACGGTCTGACAGTCCTACCTCCATTATTGACAGATGAAATGATTGCTC
AATATACATCAGCATTGCTTGCTGGCACTATCACGAGTGGATGGACTTTTGGTGCTGGCGCTGCT
TTACAAATTCCATTTGCCATGCAAATGGCTTATAGATTTAATGGTATTGGTGTTACACAAAATGT
TTTATATGAGAATCAAAAGTTAATAGCTAACCAATTTAACTCTGCAATTGGCAAGATTCAGGATT
CATTATCTAGTACAGCGAGTGCTTTAGGTAAACTACAAGATGTAGTGAATCAGAATGCTCAAGCA
CTCAATACTTTGGTTAAACAATTAAGTTCAAATTTTGGTGCAATTTCAAGTGTACTAAATGATAT
TCTAAGTCGCTTAGATAAAGTTGAGGCTGAAGTACAAATCGATAGACTAATTACAGGTAGATTAC
AGTCATTACAAACTTATGTTACTCAACAGTTAATTAGAGCTGCAGAAATAAGAGCATCTGCAAAT
TTGGCAGCCACTAAGATGAGTGAGTGTGTCCTTGGACAATCAAAACGTGTAGATTTTTGCGGAAA
GGGATATCACTTAATGTCATTTCCGCAATCTGCACCTCATGGTGTCGTGTTTCTTCATGTTACTT
ACGTTCCGGCTCAAGAGAAAAACTTCACTACGGCTCCAGCGATTTGTCATGATGGTAAAGCTCAT
TTTCCTCGTGAGGGTGTATTTGTATCAAATGGAACACATTGGTTTGTTACTCAAAGAAATTTTTA
TGAGCCACAAATAATAACTACAGATAATACTTTTGTTAGCGGTAACTGTGACGTAGTTATAGGAA
TCGTAAACAACACAGTGTATGATCCATTACAACCAGAGTTAGATTCTTTTAAAGAAGAACTTGAT
AAGTATTTCAAAAATCATACTAGCCCTGATGTTGACCTTGGTGACATATCAGGCATAAATGCATC
AGTTGTTAATATTCAAAAAGAAATAGATAGGCTTAATGAAGTTGCTAAAAATCTTAATGAATCTT
TAATAGATCTACAAGAACTTGGAAAATACGAACAATATATAAAATGGCCTTGGTATATATGGTTA
GGGTTTATTGCTGGTCTTATTGCTATTGTAATGGTAACTATTATGCTATGTTGTATGACATCATG
CTGTAGCTGTCTAAAGGGTTGTTGTAGTTGTGGTTCATGTTGCAAATTTGATGAAGATGATAGTG
AGCCAGTTCTTAAAGGTGTAAAATTGGGGGGATCTGGAATGTACAGCTTTGTGTCAGAAGAAACC
GGTACACTAATTGTTAATAGCGTTTTACTTTTTCTGGCTTTTGTTGTGTTTCTTCTAGTAACATT
GGCCATCTTGACTGCACTAAGACTTTGTGCTTATTGCTGTAATATTGTTAATGTTTCATTAGTAA
AACCTAGCTTTTATGTTTATTCGAGAGTCAAAAACCTAAATTCCAGTAGAGTACCTGATTTATTA
GTATAAggttaaggatccactagctcgtttcaaattaccgatgatatcggaccgttccaacttac
cgaccagttcggcaggtatgtatttgcgtgcattcctatccaaaaaaacatcaagccaaaagctt
gaaaaaacttacaacacagctcaacagagctagattgtaaaaccctgctttgttaagcagaacgc
aaaaattgaatgacttatagtcatategcttcgaccctegtagattagtagccttgagctattaa
ctggttgaaacacttaccaaataaagattaaaagcgataaaaatgaaagataaagcagctaaaaa
cagagattttagaaagactattttatcagtgttacaacgcaataaagatggctcttttgctacgc
aagcaaatagaaagtctattctgttgcaggcaactaaagaccttaaaaaggtagggtttagcaag
gttacagccgaaaacttcggtaataagcattgctatgcacttagagaccattggagagcccaagg
attagctacagcaacgataaaaaatcgtttagcttgtctaaggtggttaggcgagaaaatgggca
aagaactacccgataatcgaaaattagagattgagaacaggaagtatagcgataattcaatcaat
aaagcccaagaaatcgattttaaggcgatttctgccttaactgataggcaagccctagcaataca
attacagcgcgaatttgggcttcgtagagaagaaagtttgaagtttcagcctagttatgcaatca
aagagcataaaatcgagcttaaaagctcttggacaaagggtggaagaccacgagaaatcccaatt
ttgaatgaaaaacagagagaattgttagaaaaagtaaaagaggtagcaggtaaaggctctctaat
tgagagcgaaaagtcttataagcaagcaatggaacatttcacgactcgctgtcaaagagcaggga
ttaagaatgttcatggctttagacatgcgtatgctcaagatagatataggcaattaacagggcgt
gagtgtcctaaaaatggtggattaacatctaagcagctaacacctgagcaaaagcaacaagacta
tgaagctagaatgactattagtgaagagttaggtcatggtagagaagatgtaacagtcaactact
taggcagataaaaagcaatatagctatagaagaaaagaaagctattttacatagtagatcgactc
ttcttagggattttatattttttgataaatcatctattttgctagttaaatcatcaaatttatca
tcttgttgtttgactaaatctaagaatctattctcttttttaaaatcgttcatgcaaaccgccta
tagctttcttctttttctgaaattatttgtcttcacaccataattaaattcccatttttataagt
aaagtcttttaaaagcttgtcagtctcttctctagaaatgtaccaaattttacctattttaggat
acttttcatgaagTtcttctatttttccccagtcctttaatagtctacctttagagtctcgtaaa
tagttatctttgtgacaggggcctcttttatcttttttaatgtaactatatgttattccaacgtc
actattactattatccaaatcttttttagcatgccagtaagaactttcataacttaactctatct
ttcgacctctttgatatacaacaataaagctatagccagtagtaacaacctgttttacttttgtt
aaatctattaacttcttatttatttttttatgtttttttgaaaatttaaatatttctatattcat
tcctacacttcctcaaatccaaatggtagcttatgattctcttctggtttcttttctaatttttt
tatatttgcaataaaaactctttttctatctttgatttttttattgtcccaattcctccaagtat
catcacaaaccctttcaatatcatgtaaatgatgatgtctaaatattgatctgacataatacaga
tctaggtctagttcatcacttaacacaacttctctaagtctttcagatgcttcgattggtatgta
atcctctttatttttagtatctaaaagcttttgcttaaattcttcttctgtctctgctaccttac
taactgtaaacttgatatttgtaatcttacgaccatgttttctgtgatgatccttgtcatcatag
gttacaaaaatatccgataattgattaatctcttctagtgctggtaataggaacttatttttaaa
atttgaatatctgttgctgtaacttttaggtaaatcaaaatcattaatcatatcatcgacataca
atacgcaatcaactatattagcataccctgcttgttegcctaatttgcttttgagaagtaagtat
aatctgcttgaatacttacttttaaatgaaaatagtaactgtctttctgctttagtaaagtactc
ttgtagttgtatcatgtgtggcattaatgaccaatgaaactcgcaaattaaagcactgcttttag
ggtctgcttcaatatatgcaaaccagttagctatcttcgtttgttctttattcagccatactggc
ttagacattattgagtgcattaattgcttcaatctcactctgttatgcttaacccctgtagcttt
ttcaagatcagataggcttatcttatacctgtgaaactctttatcttctcttttaaccattgagg
caactaagaatattaagttttgttcttcttttgtaaggctatactttcctgcaacaagagtatta
gacatagctatttctttgccagcatttacatttttaacttctttcatagaactagagtcattatc
tcgatatacaaattctataaaacttctattagtaaaacaactacttcataaaaaaaagtagtttt
aacgatacaaaaagtagttttaaattcaaaaagtgatacaaaaagtagttttaaattcaaaaagt
gatacaaaaagtagttttaaattttttaaaaaagtgcttcaaagccttatgtagcaatacttaca
gaggattaaaaaaaaatctgacaatatataaagagaatatataaagagaatatcttaggggattt
taaaaaaatcccacagactcaaagacttttttgactttttaaatcctagaaactatactttaagt
acttatttaagtacatggatttagattatgcaaaccgttaattattcaacttttagaaatgaact
atctgattcaatggatagagtaacaaaaaatcatagtcctatgattgtaactagaggttcaaaaa
aagaagcagttgttatgatgtcgttagaggattcttcccttcctttctcgccacgttcgccggct
ttccccgtcaagctctaaatcgggggctccctttagggttccgatttagtgctttacggcacctc
gaccccaaaaaacttgattagggtgatggttcacgtagtgggccatcgccctgatagacggtttt
tcgccctttgacgttggagtccacgttctttaatagtggactcttgttccaaactggaacaacac
tcaaccctatctcggtctattcttttgatttataagggattttgccgatttcggcctattggtta
aaaaatgagctgatttaacaaaaatttaacgcgaattttaacaaaattcagggcgcaagggctgc
taaaggaagcggaacacgtagaaagccagtccgcagaaacggtgctgaccccggatgaatgtcag
ctactgggctatctggacaagggaaaacgcaagcgcaaagagaaagcaggtagcttgcagtgggc
ttacatggcgatagctagactgggcggttttatggacagcaagcgaaccggaattgccagctggg
gcgccctctggtaaggttgggaagccctgcaaagtaaactggatggctttcttgccgccaaggat
ctgatggcgcaggggatcaagatctgatcaagagacaggatgaggatcgtttcgcatgattgaac
aagatggattgcacgcaggttctccggccgcttgggtggagaggctattcggctatgactgggca
caacagacaatcggctgctctgatgccgccgtgttccggctgtcagcgcaggggcgcccggttct
ttttgtcaagaccgacctgtccggtgccctgaatgaactgcaggacgaggcagcgcggctatcgt
ggctggccacgacgggcgttccttgcgcagctgtgctcgacgttgtcactgaagcgggaagggac
tggctgctattgggcgaagtgccggggcaggatctcctgtcatcccaccttgctcctgccgagaa
agtatccatcatggctgatgcaatgcggcggctgcatacgcttgatccggctacctgcccattcg
accaccaagcgaaacatcgcatcgagcgagcacgtactcggatggaagccggtcttgtcgatcag
gatgatctggacgaagaAcatcaggggctcgcgccagccgaactgttcgccaggctcaaggcgcc
catgcccgacggcgaggatctcgtcgtgacccatggcgatgcctgcttgccgaatatcatggtgg
aaaatggccgcttttctggattcatcgactgtggccggctaggtgtggcggaccgctatcaggac
ataccattcgctacccataatattcctaaacaActtcccaccaaatggcctaaccacttcctcat
gctttacggtatcgccgctcccgattcgcagcgcatcgccttctatcgccttcttgacgagttct
tctaaactatcacaccaaatttactcatatatactttacattaatttaaaacttcatttttaatt
taaaaggatctaggtgaagatcctttttgataatctcatgaccaaaatcccttaacgtgagtttt
cgttccactgagcgtcagaccccgtagaaaagatcaaaggatcttcttgagatcctttttttctg
cgcgtaatctgctgcttgcaaacaaaaaaaccaccgctaccagcggtggtttatttgccggatca
agagctaccaactctttttccgaaggtaactggcttcagcagagcgcagataccaaatactgttc
ttctagtgtagccgtagttaggccaccacttcaagaactctgtagcaccgcctacatacctcgct
ctgctaatcctgttaccagtggctgctgccagtggcgataagtcgtgtcttaccgggttggactc
aagacgatagttaccggataaggcgcagcggtcgggctgaacggggggttcgtgcacacagccca
gcttggagcgaacgacctacaccgaactgagatacctacagcgtgagctatgagaaagcgccacg
cttcccgaagggagaaaggcggacaggtatccggtaagcggcaggatcggaacaggagagcgcac
gagggagcttccagggggaaacgcctggtatctttatagtcctgtcgggtttcgccacctctgac
ttgagcgtcgatttttgtgatgctcgtcaggggggcggagcctatggaaaaacgccagcaacgcc
gcctttttacggttcctggccttttgctggccttttgctcacatgttctttcctgcgttatcccc
tgattctgtggataaccgtattaccgcctttgagtgagctgataccactcgccgcagccgaacga
ccgagcgcagcgagtcagtgagcgaggaagcggaaAagcgcccaatacgcaaaccgcctctcccc
gcgcgttggccgattcattaatgcagctggcacgacaggtttcccgactggaaagcgggcagtga
gcgcaacgcaattaatgtgagttagctcactcattaggcaccccaggctttacactttatgcttc
cggctcgtatgttgtgtggaattgtgagcggataacaatttcacacaggaaacagctatgaccat
gattacgccaagctt (SEQ ID NO: 8)
9. pFNLdAp-bfr-N3F8H-SCcV2_(SdTM) (10029 bp)
TGAggttaaggatccactagctcgtttcaaattaccgatgatatcggaccgttccaacttaccga
ccagttcggcaggtatgtatttgcgtgcattcctatccaaaaaaacatcaagccaaaagcttgaa
aaaacttacaacacagctcaacagagctagattgtaaaaccctgctttgttaagcagaacgcaaa
aattgaatgacttatagtcatatcgcttcgaccctcgtagattagtagccttgagctattaactg
gttgaaacacttaccaaataaagattaaaagcgataaaaatgaaagataaagcagctaaaaacag
agattttagaaagactattttatcagtgttacaacgcaataaagatggctcttttgctacgcaag
caaatagaaagtctattctgttgcaggcaactaaagaccttaaaaaggtagggtttagcaaggtt
acagccgaaaacttcggtaataagcattgctatgcacttagagaccattggagagcccaaggatt
agctacagcaacgataaaaaatcgtttagcttgtctaaggtggttaggcgagaaaatgggcaaag
aactacccgataatcgaaaattagagattgagaacaggaagtatagcgataattcaatcaataaa
gcccaagaaatcgattttaaggcgatttctgccttaactgataggcaagccctagcaatacaatt
acagcgcgaatttgggcttcgtagagaagaaagtttgaagtttcagcctagttatgcaatcaaag
agcataaaatcgagcttaaaagctcttggacaaagggtggaagaccacgagaaatcccaattttg
aatgaaaaacagagagaattgttagaaaaagtaaaagaggtagcaggtaaaggctctctaattga
gagcgaaaagtcttataagcaagcaatggaacatttcacgactcgctgtcaaagagcagggatta
agaatgttcatggctttagacatgcgtatgctcaagatagatataggcaattaacagggcgtgag
tgtcctaaaaatggtggattaacatctaagcagctaacacctgagcaaaagcaacaagactatga
agctagaatgactattagtgaagagttaggtcatggtagagaagatgtaacagtcaactacttag
gcagataaaaagcaatatagctatagaagaaaagaaagctattttacatagtagatcgactcttc
ttagggattttatattttttgataaatcatctattttgctagttaaatcatcaaatttatcatct
tgttgtttgactaaatctaagaatctattctcttttttaaaatcgttcatgcaaaccgcctatag
ctttcttctttttctgaaattatttgtcttcacaccataattaaattcccatttttataagtaaa
gtcttttaaaagcttgtcagtctcttctctagaaatgtaccaaattttacctattttaggatact
tttcatgaagTtcttctatttttccccagtcctttaatagtctacctttagagtctcgtaaatag
ttatctttgtgacaggggcctcttttatcttttttaatgtaactatatgttattccaacgtcact
attactattatccaaatcttttttagcatgccagtaagaactttcataacttaactctatctttc
gacctctttgatatacaacaataaagctatagccagtagtaacaacctgttttacttttgttaaa
tctattaacttcttatttatttttttatgtttttttgaaaatttaaatatttctatattcattcc
tacacttcctcaaatccaaatggtagcttatgattctcttctggtttcttttctaatttttttat
atttgcaataaaaactctttttctatctttgatttttttattgtcccaattcctccaagtatcat
cacaaaccctttcaatatcatgtaaatgatgatgtctaaatattgatctgacataatacagatct
aggtctagttcatcacttaacacaacttctctaagtctttcagatgcttcgattggtatgtaatc
ctctttatttttagtatctaaaagcttttgcttaaattcttcttctgtctctgctaccttactaa
ctgtaaacttgatatttgtaatcttacgaccatgttttctgtgatgatccttgtcatcataggtt
acaaaaatatccgataattgattaatctcttctagtgctggtaataggaacttatttttaaaatt
tgaatatctgttgctgtaacttttaggtaaatcaaaatcattaatcatatcatcgacatacaata
cgcaatcaactatattagcataccctgcttgttcgcctaatttgcttttgagaagtaagtataat
ctgcttgaatacttacttttaaatgaaaatagtaactgtctttctgctttagtaaagtactcttg
tagttgtatcatgtgtggcattaatgaccaatgaaactcgcaaattaaagcactgcttttagggt
ctgcttcaatatatgcaaaccagttagctatcttcgtttgttctttattcagccatactggctta
gacattattgagtgcattaattgcttcaatctcactctgttatgcttaacccctgtagctttttc
aagatcagataggcttatcttatacctgtgaaactctttatcttctcttttaaccattgaggcaa
ctaagaatattaagttttgttcttcttttgtaaggctatactttcctgcaacaagagtattagac
atagctatttctttgccagcatttacatttttaacttctttcatagaactagagtcattatctcg
atatacaaattctataaaacttctattagtaaaacaactacttcataaaaaaaagtagttttaac
gatacaaaaagtagttttaaattcaaaaagtgatacaaaaagtagttttaaattcaaaaagtgat
acaaaaagtagttttaaattttttaaaaaagtgcttcaaagccttatgtagcaatacttacagag
gattaaaaaaaaatctgacaatatataaagagaatatataaagagaatatcttaggggattttaa
aaaaatcccacagactcaaagacttttttgactttttaaatcctagaaactatactttaagtact
tatttaagtacatggatttagattatgcaaaccgttaattattcaacttttagaaatgaactatc
tgattcaatggatagagtaacaaaaaatcatagtcctatgattgtaactagaggttcaaaaaaag
aagcagttgttatgatgtcgttagaggattcttcccttcctttctcgccacgttcgccggctttc
cccgtcaagctctaaatcgggggctccctttagggttccgatttagtgctttacggcacctcgac
cccaaaaaacttgattagggtgatggttcacgtagtgggccatcgccctgatagacggtttttcg
ccctttgacgttggagtccacgttctttaatagtggactcttgttccaaactggaacaacactca
accctatctcggtctattcttttgatttataagggattttgccgatttcggcctattggttaaaa
aatgagctgatttaacaaaaatttaacgcgaattttaacaaaattcagggcgcaagggctgctaa
aggaagcggaacacgtagaaagccagtccgcagaaacggtgctgaccccggatgaatgtcagcta
ctgggctatctggacaagggaaaacgcaagcgcaaagagaaagcaggtagcttgcagtgggctta
catggcgatagctagactgggcggttttatggacagcaagcgaaccggaattgccagctggggcg
ccctctggtaaggttgggaagccctgcaaagtaaactggatggctttcttgccgccaaggatctg
atggcgcaggggatcaagatctgatcaagagacaggatgaggatcgtttcgcatgattgaacaag
atggattgcacgcaggttctccggccgcttgggtggagaggctattcggctatgactgggcacaa
cagacaatcggctgctctgatgccgccgtgttccggctgtcagcgcaggggcgcccggttctttt
tgtcaagaccgacctgtccggtgccctgaatgaactgcaggacgaggcagcgcggctatcgtggc
tggccacgacgggcgttccttgcgcagctgtgctcgacgttgtcactgaagcgggaagggactgg
ctgctattgggcgaagtgccggggcaggatctcctgtcatcccaccttgctcctgccgagaaagt
atccatcatggctgatgcaatgcggcggctgcatacgcttgatccggctacctgcccattcgacc
accaagcgaaacatcgcatcgagcgagcacgtactcggatggaagccggtcttgtcgatcaggat
gatctggacgaagaAcatcaggggctcgcgccagccgaactgttcgccaggctcaaggcgcgcat
gcccgacggcgaggatctcgtcgtgacccatggcgatgcctgcttgccgaatatcatggtggaaa
atggccgcttttctggattcatcgactgtggccggctgggtgtggcggaccgctatcaggacata
gcgttggctacccgtgatattgctgaagaActtggcggcgaatgggctgaccgcttcctcgtgct
ttacggtatcgccgctcccgattcgcagcgcatcgccttctatcgccttcttgacgagttcttct
gaactgtcagaccaagtttactcatatatactttagattgatttaaaacttcatttttaatttaa
aaggatctaggtgaagatcctttttgataatctcatgaccaaaatcccttaacgtgagttttcgt
tccactgagcgtcagaccccgtagaaaagatcaaaggatcttcttgagatcctttttttctgcgc
gtaatctgctgcttgcaaacaaaaaaaccaccgctaccagcggtggtttgtttgccggatcaaga
gctaccaactctttttccgaaggtaactggcttcagcagagcgcagataccaaatactgttcttc
tagtgtagccgtagttaggccaccacttcaagaactctgtagcaccgcctacatacctcgctctg
ctaatcctgttaccagtggctgctgccagtggcgataagtcgtgtcttaccgggttggactcaag
acgatagttaccggataaggcgcagcggtcgggctgaacggggggttcgtgcacacagcccagct
tggagcgaacgacctacaccgaactgagatacctacagcgtgagctatgagaaagcgccacgctt
cccgaagggagaaaggcggacaggtatccggtaagcggcagggtcggaacaggagagcgcacgag
ggagcttccagggggaaacgcctggtatctttatagtcctgtcgggtttcgccacctctgacttg
agcgtcgatttttgtgatgctcgtcaggggggcggagcctatggaaaaacgccagcaacgcggcc
tttttacggttcctggccttttgctggccttttgctcacatgttctttcctgcgttatcccctga
ttctgtggataaccgtattaccgcctttgagtgagctgataccgctcgccgcagccgaacgaccg
agcgcagcgagtcagtgagcgaggaagcggaaAagcgcccaatacgcaaaccgcctctccccgcg
cgttggccgattcattaatgcagctggcacgacaggtttcccgactggaaagcgggcagtgagcg
caacgcaattaatgtgagttagctcactcattaggcaccccaggctttacactttatgcttccgg
ctcgtatgttgtgtggaattgtgagcggataacaatttcacacaggaaacagctatgaccatgat
tacgccaagcttggtacctggttactattgccatcatcacaatattaaaattaattttcttcatt
tatttttcttaaatattattattaaaaatagtaaatttaacttatctaaaaatagcataatatca
tttttattaaaatatctaggttgaattcttagatattttgatatataattagatactaaattgag
aacttataaagaattaaattttcttttgtatgctaacttgattgctaatatgaattatactagtt
agtatgttgattataatgattagagttttaaataatggaggtaacaataggaggtacgtaatgga
ttataaagatcacgatggtgattacaaagaccatgatatagattataaggatgacgatgataagc
atcatcatcaccaccatcatcatggaggtggttcaATGTTTGTGTTTTTAGTTCTTTTACCGTTA
GTTTCAAGTCAATGTGTGAACTTAACTACACGCACACAACTTCCTCCAGCATATACAAATAGTTT
TACTAGAGGTGTATATTATCCTGATAAAGTATTCCGTAGTTCTGTTCTACATTCTACACAAGATT
TGTTTTTACCGTTTTTCAGTAATGTCACTTGGTTCCATGCTATTCATGTTTCTGGGACAAACGGT
ACAAAAAGATTTGATAACCCTGTTTTACCATTTAATGATGGTGTATATTTTGCTTCAACTGAGAA
AAGCAATATAATTAGAGGTTGGATTTTCGGAACTACCCTGGATAGCAAGACGCAAAGTTTATTGA
TCGTAAACAATGCTACAAACGTCGTAATTAAAGTATGTGAATTTCAATTTTGTAATGACCCTTTT
TTAGGAGTCTATTATCATAAAAATAATAAATCTTGGATGGAGTCTGAATTTAGAGTTTATTCTAG
CGCTAATAACTGTACATTTGAATATGTTTCACAACCTTTTTTAATGGATCTAGAAGGTAAACAGG
GTAATTTTAAAAATCTTCGTGAGTTTGTTTTTAAGAACATAGATGGATATTTCAAAATATATTCA
AAACATACTCCTATTAATCTAGTTAGAGATCTTCCACAAGGCTTTTCTGCTCTAGAACCATTAGT
TGATTTACCAATAGGTATAAATATAACTCGTTTCCAAACTTTACTAGCCCTTCACCGTTCGTACT
TAACGCCTGGGGATTCTTCTAGTGGTTGGACTGCTGGCGCTGCAGCATATTATGTTGGATATCTA
CAACCTAGAACATTTTTATTGAAATACAACGAAAACGGAACTATAACTGACGCTGTTGATTGTGC
ACTTGATCCATTAAGTGAGACTAAATGTACTCTAAAAAGTTTTACTGTTGAAAAGGGAATTTATC
AAACATCAAATTTTCGCGTTCAACCAACGGAAAGTATTGTACGTTTTCCGAACATAACCAATTTA
TGTCCTTTCGGTGAGGTATTTAACGCAACTCGTTTTGCGAGCGTATATGCTTGGAATAGAAAAAG
AATTAGCAATTGTGTTGCTGATTATTCGGTCTTATACAATAGTGCTTCGTTTAGCACTTTTAAAT
GTTACGGAGTAAGTCCAACAAAGTTAAATGATCTATGTTTCACTAATGTGTATGCTGATTCTTTT
GTTATTAGAGGTGATGAAGTTCGACAAATTGCTCCAGGTCAAACTGGCAAAATTGCGGACTATAA
TTATAAGCTACCTGATGATTTTACTGGCTGTGTGATTGCATGGAATAGTAATAATCTAGATTCGA
AAGTCGGTGGGAATTATAATTATCTTTATAGACTATTTAGAAAATCTAATTTGAAACCATTTGAG
AGAGATATATCAACAGAAATTTACCAGGCTGGCAGCACACCTTGCAACGGCGTAGAAGGTTTTAA
TTGTTATTTTCCACTACAAAGTTATGGTTTTCAACCAACTAATGGCGTCGGGTATCAACCATATA
GAGTTGTCGTACTTTCCTTTGAATTACTTCATGCACCAGCTACCGTTTGTGGGCCAAAGAAATCA
ACTAATCTTGTAAAGAATAAATGCGTCAATTTTAATTTTAATGGCCTTACAGGCACTGGAGTTTT
AACAGAATCCAATAAAAAATTTTTACCTTTTCAGCAATTTGGTAGAGATATAGCTGATACTACTG
ATGCTGTAAGAGATCCTCAAACTCTAGAGATTTTAGATATTACCCCGTGTTCATTTGGAGGCGTA
AGCGTTATAACTCCAGGCACGAACACATCAAATCAAGTTGCTGTACTATATCAAGATGTTAATTG
CACAGAAGTGCCTGTTGCCATTCATGCAGATCAACTTACTCCTACATGGCGTGTATATTCTACCG
GATCAAATGTATTTCAGACTAGAGCTGGTTGTTTAATAGGCGCAGAACATGTAAATAATAGTTAT
GAGTGTGATATACCAATTGGTGCAGGAATATGTGCATCATATCAGACACAGACAAATAGTCCTCG
TCGCGCAAGATCAGTAGCATCACAATCGATTATAGCTTATACAATGTCTTTAGGTGCGGAAAATA
GTGTGGCTTATTCTAATAATTCTATCGCAATCCCTACCAATTTCACTATAAGTGTTACAACCGAA
ATCTTACCAGTTAGTATGACAAAGACAAGTGTTGATTGTACTATGTATATATGTGGCGATTCTAC
TGAGTGTTCTAATCTCTTATTACAATATGGTTCGTTTTGTACTCAGTTAAATCGAGCTCTTACAG
GTATAGCTGTCGAGCAAGATAAGAATACCCAGGAAGTCTTTGCACAGGTTAAACAAATTTATAAA
ACTCCACCAATCAAAGATTTTGGTGGGTTTAACTTTTCTCAAATACTACCTGATCCATCTAAACC
CTCTAAACGTAGTTTTATTGAAGATTTACTTTTTAATAAAGTAACTCTAGCTGATGCTGGTTTCA
TTAAACAATACGGCGATTGTTTGGGTGATATAGCGGCACGTGATTTAATATGCGCACAGAAATTC
AACGGTCTGACAGTCCTACCTCCATTATTGACAGATGAAATGATTGCTCAATATACATCAGCATT
GCTTGCTGGCACTATCACGAGTGGATGGACTTTTGGTGCTGGCGCTGCTTTACAAATTCCATTTG
CCATGCAAATGGCTTATAGATTTAATGGTATTGGTGTTACACAAAATGTTTTATATGAGAATCAA
AAGTTAATAGCTAACCAATTTAACTCTGCAATTGGCAAGATTCAGGATTCATTATCTAGTACAGC
GAGTGCTTTAGGTAAACTACAAGATGTAGTGAATCAGAATGCTCAAGCACTCAATACTTTGGTTA
AACAATTAAGTTCAAATTTTGGTGCAATTTCAAGTGTACTAAATGATATTCTAAGTCGCTTAGAT
CCTCCAGAGGCTGAAGTACAAATCGATAGACTAATTACAGGTAGATTACAGTCATTACAAACTTA
TGTTACTCAACAGTTAATTAGAGCTGCAGAAATAAGAGCATCTGCAAATTTGGCAGCCACTAAGA
TGAGTGAGTGTGTCCTTGGACAATCAAAACGTGTAGATTTTTGCGGAAAGGGATATCACTTAATG
TCATTTCCGCAATCTGCACCTCATGGTGTCGTGTTTCTTCATGTTACTTACGTTCCGGCTCAAGA
GAAAAACTTCACTACGGCTCCAGCGATTTGTCATGATGGTAAAGCTCATTTTCCTCGTGAGGGTG
TATTTGTATCAAATGGAACACATTGGTTTGTTACTCAAAGAAATTTTTATGAGCCACAAATAATA
ACTACAGATAATACTTTTGTTAGCGGTAACTGTGACGTAGTTATAGGAATCGTAAACAACACAGT
GTATGATCCATTACAACCAGAGTTAGATTCTTTTAAAGAAGAACTTGATAAGTATTTCAAAAATC
ATACTAGCCCTGATGTTGACCTTGGTGACATATCAGGCATAAATGCATCAGTTGTTAATATTCAA
AAAGAAATAGATAGGCTTAATGAAGTTGCTAAAAATCTTAATGAATCTTTAATAGATCTACAAGA
ACTTGGAAAATACGAACAA (SEQ ID NO: 9)

1. An immunogenic composition comprising:
a Francisella tularensis subspecies holarctica Live Vaccine Strain (LVS):
having a deletion in a capB gene; and
expressing at least one antigenic polypeptide epitope present in a polypeptide expressed by severe acute respiratory syndrome coronavirus 2 (SARS-CoV-2);
wherein:
the antigenic polypeptide epitope elicits an immune response to SARS-CoV-2 in a mammalian host when the immunogenic composition is administered orally (p.o.), intradermally (i.d.), subcutaneously (s.q.), intramuscularly (i.m.), intranasally (i.n.) or by inhalation to the mammalian host.

2. The immunogenic composition of claim 1, wherein the at least one antigenic polypeptide epitope present in the polypeptide expressed by severe acute respiratory syndrome coronavirus 2 is present on SARS-CoV-2 membrane (M) glycoprotein; and/or SARS-CoV-2 nucleocapsid (N) phosphoprotein.

3. The immunogenic composition of claim 2, wherein the LVS expresses a fusion protein comprising at least one peptide epitope present in SARS-CoV-2 membrane (M) glycoprotein and at least one peptide epitope present in SARS-CoV-2 nucleocapsid (N) phosphoprotein.

4. The immunogenic composition of claim 3, wherein the fusion protein is encoded by SEQ ID NO: 1.

5. The immunogenic composition of claim 3, wherein the at least two antigenic polypeptide epitopes are encoded by a polynucleotide sequence that is at least 50, 100, 200, 300 or 400 nucleotides in length.

6. The immunogenic composition of claim 2, wherein the antigenic polypeptide epitope is encoded by a codon optimized polynucleotide sequence.

7. The immunogenic composition of claim 1, further comprising a pharmaceutical excipient adapted for oral administration.

8. A method of making an immunogenic composition, the method comprising:
introducing a polynucleotide encoding at least one antigenic epitope present in a polypeptide expressed by severe acute respiratory syndrome coronavirus 2 (SARS-CoV-2) into a recombinant attenuated Francisella tularensis subspecies holarctica Live Vaccine Strain (LVS), wherein:
the LVS has a deletion in a capB gene; and
the antigenic polypeptide epitope encoded by the polynucleotide elicits an immune response to SARS-CoV-2 in a mammalian host when the immunogenic composition is administered intranasally to the mammalian host.

9. The method of claim 8, wherein the at least one antigenic polypeptide epitope present in the polypeptide expressed by severe acute respiratory syndrome coronavirus 2 is present on SARS-CoV-2 membrane (M) glycoprotein; or SARS-CoV-2 nucleocapsid (N) phosphoprotein.

10. The method of claim 9, wherein the LVS expresses at least two antigenic polypeptide epitopes including: at least one peptide epitope present in SARS-CoV-2 membrane (M) glycoprotein; at least one peptide epitope present in SARS-CoV-2 nucleocapsid (N) phosphoprotein.

11. The method of claim 10, wherein the at least two antigenic polypeptide epitopes present on a severe acute respiratory syndrome coronavirus 2 polypeptide are encoded by SEQ ID NO: 1.

12. The method of claim 11, wherein the at least two antigenic polypeptide epitopes present on a severe acute respiratory syndrome coronavirus 2 polypeptide are encoded by a polynucleotide sequence that is at least 50, 100, 200, 300 or 400 nucleotides in length.

13. The method of claim 8, wherein the antigenic polypeptide is encoded in a codon optimized polynucleotide sequence.

14. The method of claim 8, further comprising combining the LVS with a pharmaceutical excipient adapted for oral or intranasal administration.

15. A method of generating an immune response in a mammal comprising administering the immunogenic composition of any one of claim 1 to the mammal so that an immune response is generated to the antigenic polypeptide epitope present in a severe acute respiratory syndrome coronavirus 2 polypeptide.

16. The method of claim 15, wherein the method comprises administering the immunogenic composition of claim 1 in a primary vaccination; and administering the immunogenic composition of claim 1 in a subsequent homologous booster vaccination one or more times.

17. The method of claim 15, wherein method comprises administering a single dose of the composition of claim 1, and one or more doses of a second immunogenic composition.

18. The method of claim 15, wherein the immunogenic composition is administered orally.

19. The method of claim 15, wherein the immunogenic composition is administered intranasally.

20. Use of the immunogenic composition of any one of claim 1 for the inducing immunity to SARS-CoV-2.
